SEQUENCE LISTING (115 sequences)

SEQ ID NO: 1 - protein, 5 aa, synthetic construct
NYVMH

SEQ ID NO: 2 - protein, 17 aa, synthetic construct
YINPYNDDVKYNEKFKG

SEQ ID NO: 3 - protein, 13 aa, synthetic construct
WGYYGSPLYYFDY

SEQ ID NO: 4 - protein, 10 aa, synthetic construct
RASSRLIYMH

SEQ ID NO: 5 - protein, 7 aa, synthetic construct
ATSNLAS

SEQ ID NO: 6 - protein, 9 aa, synthetic construct
QQWNSNPPT

SEQ ID NO: 7 - protein, 122 aa, synthetic construct
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 8 - protein, 107 aa, synthetic construct
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELKR

SEQ ID NO: 9 - DNA, 366 bp, synthetic construct
  source 1..366: mol_type = other DNA
gaagtacagcttcagcagtcaggtccagaactggtcaagccaggagcatccgtaaagatgtcctgtaaggccagcggttacacatttacgaattacgtcatgcattgggtaaagcagaagccaggccaaggtcttgaatggatcggatacattaacccatacaatgacgatgtgaaatacaatgagaagtttaagggcaaagctactcaaacgtcagataaatcttccagcacagcttacatggagctgtcttccctgacaagcgaggatagcgcagtttattactgcgcccgatggggttattatgggtcacctctttattattttgactattggggtcaagggacgaccttgaccgtatcatca

SEQ ID NO: 10 - DNA, 321 bp, synthetic construct
  source 1..321: mol_type = other DNA
cagatcgtgttgagtcagagtcctaccatattgtcagcgagtccgggagagaaggtaactatgacctgtcgggcttctagccggctgatatatatgcactggtaccaacaaaagccgggctcatcacctaaaccttggatatatgctacaagtaatcttgcgtcaggcgtcccagcccggttctccggctccggttcagggacaagttactcactgactataagtcgcgttgaagccgaggatgccgccacgtattattgtcagcaatggaacagtaaccctcccacatttggcacggggactaagctggaattgaaaagg

SEQ ID NO: 11 - protein, 5 aa, synthetic construct
SFGMH

SEQ ID NO: 12 - protein, 17 aa, synthetic construct
YISSDSSAIYYADTVKG

SEQ ID NO: 13 - protein, 13 aa, synthetic construct
GRENIYYGSRLDY

SEQ ID NO: 14 - protein, 11 aa, synthetic construct
KASQNVDTNVA

SEQ ID NO: 15 - protein, 7 aa, synthetic construct
SASYRYS

SEQ ID NO: 16 - protein, 9 aa, synthetic construct
QQYNNYPFT

SEQ ID NO: 17 - protein, 122 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 18 - protein, 108 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKR

SEQ ID NO: 19 - DNA, 366 bp, synthetic construct
  source 1..366: mol_type = other DNA
gaggtccagcttgttgaatcagggggcggcctcgtccaaccggggggctcactgcgcttgtcttgcgctgcctctggatttacctttagttctttcggaatgcattgggtgcggcaagcacccggcaaggggttggagtgggtagcgtatataagcagcgactcctctgccatctactacgcggatacggtgaaaggcagatttacgattagtagagacaacgctaagaattcattgtacttgcaaatgaacagcctcagagacgaggataccgccgtgtactattgtggaaggggcagggagaatatatactatgggtcaagattggattattggggacaggggacgacagtcacggtgagcagt

SEQ ID NO: 20 - DNA, 324 bp, synthetic construct
  source 1..324: mol_type = other DNA
gacattcagctcactcagtcaccatcttttctgtcagcgtctgtgggggacagagtaacgataacgtgcaaagcgagtcagaacgttgataccaatgtggcctggtatcagcaaaagcccggtaaagccccgaaggcgctgatctattctgccagctaccgatactcaggcgttccttcacgctttagcgggtcaggctctggtaccgacttcactcttacgatcagttcattgcagcccgaagacttcgccacttactactgccaacaatataataactaccctttcacgtttggccaaggcaccaagttggagattaagcgg

SEQ ID NO: 21 - protein, 5 aa, synthetic construct
SYAMS

SEQ ID NO: 22 - protein, 17 aa, synthetic construct
AISGSGGSTYYADSVKG

SEQ ID NO: 23 - protein, 8 aa, synthetic construct
NYISMFDS

SEQ ID NO: 24 - protein, 11 aa, synthetic construct
GGNNIGSKSVH

SEQ ID NO: 25 - protein, 7 aa, synthetic construct
YDSDRPS

SEQ ID NO: 26 - protein, 10 aa, synthetic construct
QVWDSSSDYV

SEQ ID NO: 27 - protein, 117 aa, synthetic construct
EVQLVESGGGLIQPGGSLRLSCAASGFTFSSYAMSWVRQAPGKGLEWVSAISGSGGSTYYADSVKGRFTISRDNSKNTLYLQMNSLRAEDTAVYYCARNYISMFDSWGQGTLVTVSS

SEQ ID NO: 28 - protein, 108 aa, synthetic construct
QSVLTQPPSVSVAPGKTARITCGGNNIGSKSVHWYQQRPGQAPVLVIYYDSDRPSGIPERFSGSNSGNTATLTISRVEAGDEADYYCQVWDSSSDYVFGIGTKVTVLG

SEQ ID NO: 29 - DNA, 351 bp, synthetic construct
  source 1..351: mol_type = other DNA
gaggtgcagctggtggagtctggaggaggcttgatccagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgcgcaactacatctctatgttcgattcttggggtcaaggtactctggtgaccgtctcctca

SEQ ID NO: 30 - DNA, 324 bp, synthetic construct
  source 1..324: mol_type = other DNA
cagtctgtgctgactcagccaccctcagtgtcagtggccccaggaaagacggccaggattacctgtgggggaaacaacattggaagtaaaagtgtgcactggtaccagcagaggccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgattatgtcttcggaattgggaccaaggtcaccgtcctaggt

SEQ ID NO: 31 - protein, 19 aa, synthetic construct
GGGGSGGGGSGGGSGGGGS

SEQ ID NO: 32 - protein, 15 aa, synthetic construct
GGGGSGGGGSGGGGS

SEQ ID NO: 33 - protein, 24 aa, synthetic construct
GGGGSGGGGSGGGGSGGGSGGGGS

SEQ ID NO: 34 - protein, 29 aa, synthetic construct
GGGGSGGGGSGGGGSGGGGSGGGSGGGGS

SEQ ID NO: 35 - protein, 5 aa, synthetic construct
GGGGS

SEQ ID NO: 36 - protein, 10 aa, synthetic construct
GGGGSGGGGS

SEQ ID NO: 37 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 38 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 40 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 41 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 42 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 45 - protein, 4 aa, synthetic construct
YMNM

SEQ ID NO: 46-47 - skipped (2 entries)

SEQ ID NO: 48 - protein, 4 aa, synthetic construct
YENV

SEQ ID NO: 49 - protein, 4 aa, synthetic construct
YSNV

SEQ ID NO: 50 - protein, 4 aa, synthetic construct
YKNL

SEQ ID NO: 51 - protein, 4 aa, synthetic construct
YENQ

SEQ ID NO: 52 - protein, 4 aa, synthetic construct
YKNI

SEQ ID NO: 53 - protein, 4 aa, synthetic construct
YINQ

SEQ ID NO: 54 - protein, 4 aa, synthetic construct
YHNK

SEQ ID NO: 55 - protein, 4 aa, synthetic construct
YVNQ

SEQ ID NO: 56 - protein, 4 aa, synthetic construct
YLNP

SEQ ID NO: 57 - protein, 4 aa, synthetic construct
YLNT

SEQ ID NO: 58 - protein, 4 aa, synthetic construct
YDND

SEQ ID NO: 59 - protein, 4 aa, synthetic construct
YENI

SEQ ID NO: 60 - protein, 4 aa, synthetic construct
YENL

SEQ ID NO: 61 - protein, 4 aa, synthetic construct
YKNQ

SEQ ID NO: 62 - protein, 4 aa, synthetic construct
YKNV

SEQ ID NO: 63 - protein, 4 aa, synthetic construct
YANG

SEQ ID NO: 64 - protein, 4 aa, synthetic construct
YMDM

SEQ ID NO: 65 - protein, 4 aa, synthetic construct
YMPM

SEQ ID NO: 66 - protein, 4 aa, synthetic construct
YMRM

SEQ ID NO: 67 - protein, 4 aa, synthetic construct
YMSM

SEQ ID NO: 68 - skipped

SEQ ID NO: 69 - protein, 4 aa, synthetic construct
YTHM

SEQ ID NO: 70 - protein, 4 aa, synthetic construct
YVLM

SEQ ID NO: 71 - protein, 4 aa, synthetic construct
YIAM

SEQ ID NO: 72 - protein, 4 aa, synthetic construct
YVEM

SEQ ID NO: 73 - protein, 4 aa, synthetic construct
YVKM

SEQ ID NO: 74 - protein, 4 aa, synthetic construct
YVPM

SEQ ID NO: 75 - skipped

SEQ ID NO: 76 - protein, 4 aa, synthetic construct
YMAP

SEQ ID NO: 77-78 - skipped (2 entries)

SEQ ID NO: 79 - protein, 4 aa, synthetic construct
YMNV

SEQ ID NO: 80 - protein, 4 aa, synthetic construct
YENM

SEQ ID NO: 81 - protein, 4 aa, synthetic construct
YMNQ

SEQ ID NO: 82 - protein, 4 aa, synthetic construct
YMNL

SEQ ID NO: 83 - protein, 4 aa, synthetic construct
YSNM

SEQ ID NO: 84 - skipped

SEQ ID NO: 85 - protein, 4 aa, synthetic construct
YGGG

SEQ ID NO: 86 - protein, 4 aa, synthetic construct
YAAA

SEQ ID NO: 87 - protein, 4 aa, synthetic construct
YFFF

SEQ ID NO: 88 - protein, 4 aa, synthetic construct
YETV

SEQ ID NO: 89 - protein, 4 aa, synthetic construct
YQQQ

SEQ ID NO: 90 - protein, 4 aa, synthetic construct
YHAE

SEQ ID NO: 91 - protein, 4 aa, synthetic construct
YLDL

SEQ ID NO: 92 - protein, 4 aa, synthetic construct
YLIP

SEQ ID NO: 93 - protein, 4 aa, synthetic construct
YLRV

SEQ ID NO: 94 - protein, 4 aa, synthetic construct
YTAV

SEQ ID NO: 95 - protein, 4 aa, synthetic construct
YVHV

SEQ ID NO: 96 - protein, 41 aa, synthetic construct
RSKRSRLLHSDYENVTPRRPGPTRKHYQPYAPPRDFAAYRS

SEQ ID NO: 97 - protein, 41 aa, synthetic construct
RSKRSRLLHSDYKNITPRRPGPTRKHYQPYAPPRDFAAYRS

SEQ ID NO: 98 - protein, 41 aa, synthetic construct
RSKRSRLLHSDYMDMTPRRPGPTRKHYQPYAPPRDFAAYRS

SEQ ID NO: 99 - protein, 41 aa, synthetic construct
RSKRSRLLHSDYGGGTPRRPGPTRKHYQPYAPPRDFAAYRS

SEQ ID NO: 100 - protein, 41 aa, synthetic construct
RSKRSRLLHSDYSNVTPRRPGPTRKHYQPYAPPRDFAAYRS

SEQ ID NO: 101 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 102 - protein, 497 aa, synthetic construct
MALPVTALLLPLALLLHAARPEVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSSGGGGSGGGGSGGGGSQIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELKREQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 1494 bp, synthetic construct
  source 1..1494: mol_type = other DNA
atggctctcccagtgactgccctactgcttcccctagcgcttctcctgcatgcagccaggccggaagtacagcttcagcagtcaggtccagaactggtcaagccaggagcatccgtaaagatgtcctgtaaggccagcggttacacatttacgaattacgtcatgcattgggtaaagcagaagccaggccaaggtcttgaatggatcggatacattaacccatacaatgacgatgtgaaatacaatgagaagtttaagggcaaagctactcaaacgtcagataaatcttccagcacagcttacatggagctgtcttccctgacaagcgaggatagcgcagtttattactgcgcccgatggggttattatgggtcacctctttattattttgactattggggtcaagggacgaccttgaccgtatcatcaggtggaggtggatcaggtggaggtggatctggtggaggtggatctcagatcgtgttgagtcagagtcctaccatattgtcagcgagtccgggagagaaggtaactatgacctgtcgggcttctagccggctgatatatatgcactggtaccaacaaaagccgggctcatcacctaaaccttggatatatgctacaagtaatcttgcgtcaggcgtcccagcccggttctccggctccggttcagggacaagttactcactgactataagtcgcgttgaagccgaggatgccgccacgtattattgtcagcaatggaacagtaaccctcccacatttggcacggggactaagctggaattgaaaagggaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 104 - protein, 503 aa, synthetic construct
MALPVTALLLPLALLLHAARPEVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSSGGGGSGGGGSGGGGSQIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELKREQKLISEEDLAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - DNA, 1512 bp, synthetic construct
  source 1..1512: mol_type = other DNA
atggctctcccagtgactgccctactgcttcccctagcgcttctcctgcatgcagccaggccggaagtacagcttcagcagtcaggtccagaactggtcaagccaggagcatccgtaaagatgtcctgtaaggccagcggttacacatttacgaattacgtcatgcattgggtaaagcagaagccaggccaaggtcttgaatggatcggatacattaacccatacaatgacgatgtgaaatacaatgagaagtttaagggcaaagctactcaaacgtcagataaatcttccagcacagcttacatggagctgtcttccctgacaagcgaggatagcgcagtttattactgcgcccgatggggttattatgggtcacctctttattattttgactattggggtcaagggacgaccttgaccgtatcatcaggtggaggtggatcaggtggaggtggatctggtggaggtggatctcagatcgtgttgagtcagagtcctaccatattgtcagcgagtccgggagagaaggtaactatgacctgtcgggcttctagccggctgatatatatgcactggtaccaacaaaagccgggctcatcacctaaaccttggatatatgctacaagtaatcttgcgtcaggcgtcccagcccggttctccggctccggttcagggacaagttactcactgactataagtcgcgttgaagccgaggatgccgccacgtattattgtcagcaatggaacagtaaccctcccacatttggcacggggactaagctggaattgaaaagggaacagaaactgatctccgaggaggacctcgcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 106 - protein, 498 aa, synthetic construct
MALPVTALLLPLALLLHAARPEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKREQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 1497 bp, synthetic construct
  source 1..1497: mol_type = other DNA
atggctctcccagtgactgccctactgcttcccctagcgcttctcctgcatgcagccaggccggaggtccagcttgttgaatcagggggcggcctcgtccaaccggggggctcactgcgcttgtcttgcgctgcctctggatttacctttagttctttcggaatgcattgggtgcggcaagcacccggcaaggggttggagtgggtagcgtatataagcagcgactcctctgccatctactacgcggatacggtgaaaggcagatttacgattagtagagacaacgctaagaattcattgtacttgcaaatgaacagcctcagagacgaggataccgccgtgtactattgtggaaggggcagggagaatatatactatgggtcaagattggattattggggacaggggacgacagtcacggtgagcagtggtggaggtggatcaggtggaggtggatctggtggaggtggatctgacattcagctcactcagtcaccatcttttctgtcagcgtctgtgggggacagagtaacgataacgtgcaaagcgagtcagaacgttgataccaatgtggcctggtatcagcaaaagcccggtaaagccccgaaggcgctgatctattctgccagctaccgatactcaggcgttccttcacgctttagcgggtcaggctctggtaccgacttcactcttacgatcagttcattgcagcccgaagacttcgccacttactactgccaacaatataataactaccctttcacgtttggccaaggcaccaagttggagattaagcgggaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 108 - protein, 504 aa, synthetic construct
MALPVTALLLPLALLLHAARPEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKREQKLISEEDLAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 1515 bp, synthetic construct
  source 1..1515: mol_type = other DNA
atggctctcccagtgactgccctactgcttcccctagcgcttctcctgcatgcagccaggccggaggtccagcttgttgaatcagggggcggcctcgtccaaccggggggctcactgcgcttgtcttgcgctgcctctggatttacctttagttctttcggaatgcattgggtgcggcaagcacccggcaaggggttggagtgggtagcgtatataagcagcgactcctctgccatctactacgcggatacggtgaaaggcagatttacgattagtagagacaacgctaagaattcattgtacttgcaaatgaacagcctcagagacgaggataccgccgtgtactattgtggaaggggcagggagaatatatactatgggtcaagattggattattggggacaggggacgacagtcacggtgagcagtggtggaggtggatcaggtggaggtggatctggtggaggtggatctgacattcagctcactcagtcaccatcttttctgtcagcgtctgtgggggacagagtaacgataacgtgcaaagcgagtcagaacgttgataccaatgtggcctggtatcagcaaaagcccggtaaagccccgaaggcgctgatctattctgccagctaccgatactcaggcgttccttcacgctttagcgggtcaggctctggtaccgacttcactcttacgatcagttcattgcagcccgaagacttcgccacttactactgccaacaatataataactaccctttcacgtttggccaaggcaccaagttggagattaagcgggaacagaaactgatctccgaggaggacctcgcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 110 - protein, 22 aa, synthetic construct
GSGATNFSLLKQAGDVEENPGP

SEQ ID NO: 111 - DNA, 66 bp, synthetic construct
  source 1..66: mol_type = other DNA
ggatctggagcaacaaacttctcactactcaaacaagcaggtgacgtggaggagaatcccggaccc

SEQ ID NO: 112 - protein, 810 aa, synthetic construct
MALPVTALLLPLALLLHAARPEVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSSGGGGSGGGGSGGGGSQIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELKREQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMETDTLLLWVLLLWVPGSTGQSVLTQPPSVSVAPGKTARITCGGNNIGSKSVHWYQQRPGQAPVLVIYYDSDRPSGIPERFSGSNSGNTATLTISRVEAGDEADYYCQVWDSSSDYVFGIGTKVTVLGSRGGGGSGGGGSGGGGSLEMAEVQLVESGGGLIQPGGSLRLSCAASGFTFSSYAMSWVRQAPGKGLEWVSAISGSGGSTYYADSVKGRFTISRDNSKNTLYLQMNSLRAEDTAVYYCARNYISMFDSWGQGTLVTVSSTSGQAGQHHHHHHGAYPYDVPDYAS

SEQ ID NO: 113 - DNA, 2433 bp, synthetic construct
  source 1..2433: mol_type = other DNA
atggctctcccagtgactgccctactgcttcccctagcgcttctcctgcatgcagccaggccggaagtacagcttcagcagtcaggtccagaactggtcaagccaggagcatccgtaaagatgtcctgtaaggccagcggttacacatttacgaattacgtcatgcattgggtaaagcagaagccaggccaaggtcttgaatggatcggatacattaacccatacaatgacgatgtgaaatacaatgagaagtttaagggcaaagctactcaaacgtcagataaatcttccagcacagcttacatggagctgtcttccctgacaagcgaggatagcgcagtttattactgcgcccgatggggttattatgggtcacctctttattattttgactattggggtcaagggacgaccttgaccgtatcatcaggtggaggtggatcaggtggaggtggatctggtggaggtggatctcagatcgtgttgagtcagagtcctaccatattgtcagcgagtccgggagagaaggtaactatgacctgtcgggcttctagccggctgatatatatgcactggtaccaacaaaagccgggctcatcacctaaaccttggatatatgctacaagtaatcttgcgtcaggcgtcccagcccggttctccggctccggttcagggacaagttactcactgactataagtcgcgttgaagccgaggatgccgccacgtattattgtcagcaatggaacagtaaccctcccacatttggcacggggactaagctggaattgaaaagggaacagaaactgatctccgaggaggacctcgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggagcaacaaacttctcactactcaaacaagcaggtgacgtggaggagaatcccggacccatggaaaccgacaccctgctgctgtgggtgctgctgctgtgggtgccaggatctacaggacagtctgtgctgactcagccaccctcagtgtcagtggccccaggaaagacggccaggattacctgtgggggaaacaacattggaagtaaaagtgtgcactggtaccagcagaggccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgattatgtcttcggaattgggaccaaggtcaccgtcctaggttctagaggtggtggtggtagcggcggcggcggctctggtggtggtggatccctcgagatggccgaggtgcagctggtggagtctggaggaggcttgatccagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgcgcaactacatctctatgttcgattcttggggtcaaggtactctggtgaccgtctcctcaactagtggccaggccggccagcaccatcaccatcaccatggcgcatacccgtacgacgttccggactacgcttcttag

SEQ ID NO: 114 - protein, 4 aa, synthetic construct
PRRP

SEQ ID NO: 115 - protein, 4 aa, synthetic construct
PYAP

(19) United States
(12) Patent Application Publication
(10) Pub. No.: US 2025/0332256 A1
(43) Pub. Date: Oct. 30, 2025

(54) ANTIGEN-RECOGNIZING RECEPTORS TARGETING B7-H3 AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventors: Jonathan Faisal Khan, New York, NY (US); Taha Merghoub, New York, NY (US); Renier J. Brentjens, New York, NY (US); Jedd D. Wolchok, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 19/200,121

(22) Filed: May 6, 2025

Related U.S. Application Data
(63) Continuation of application No. PCT/US23/78883, filed on Nov. 7, 2023.
(60) Provisional application No. 63/382,656, filed on Nov. 7, 2022.

(51) Int. Cl. A61K 40/11; A61K 40/31; A61K 40/42; A61P 35/00; C07K 16/28; C12N 5/0783

(52) U.S. Cl. CPC A61K 40/11; A61K 40/31; A61K 40/421; A61P 35/00; C07K 16/2827; C12N 5/0636; C07K 2317/622; C12N 2510/00

(57) ABSTRACT

The presently disclosed subject matter provides for antigen-recognizing receptors that specifically target B7-H3 and cells comprising such B7-H3-targeted antigen-recognizing receptors. The presently disclosed subject matter further provides uses of the B7-H3-targeted antigen-recognizing receptors for treatment.

### CROSS-REFERENCE TO RELATED APPLICATIONS

This application is a Continuation Application of International Patent Application No. PCT/US23/78883, filed Nov. 7, 2023, which claims priority to U.S. Provisional Application No. 63/382,656, filed Nov. 7, 2022, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on May 6, 2025, is entitled “0727341767.xml”, and is 112,109 bytes in size.

### INTRODUCTION

The presently disclosed subject matter provides methods and compositions for immunotherapies. It relates to antigen-recognizing receptors (e.g., chimeric antigen receptors (CARs)) that specifically target B7-H3, cells comprising such receptors, and methods of using such cells for treatments.

### BACKGROUND OF THE INVENTION

Cell-based immunotherapy is a therapy with curative potential for the treatment of cancer. T cells and other immune cells can be modified to target tumor antigens through the introduction of genetic material coding for artificial or synthetic receptors for antigens, termed chimeric antigen receptors (CARs), specific to selected antigens. Targeted T-cell therapy using CARs has shown recent clinical success in treating hematologic malignancies and solid tumors.

B7-H3 (CD276) is a checkpoint molecule expressed at high levels on solid tumors, including sarcomas and brain tumors. B7-H3 expression contributes to tumor immune evasion and metastatic potential (Picarda et al., Clin Cancer Res 2016; 22: 3425-31; Tekle et al., Int J Cancer 2012; 130: 2282-90) and is correlated with poor prognosis (Ye et al., Cell Physiol Biochem 2016; 39:1568-80).

Given the significant role of B7-H3 in various diseases or disorders, immunotherapies (e.g., CARs) targeting B7-H3, are desired.

### SUMMARY OF THE INVENTION

The presently disclosed subject matter provides antigen-recognizing receptors (e.g., chimeric antigen receptors (CARs)) that specifically target B7-H3. The presently disclosed subject matter further provides cells comprising such receptors.

In certain non-limiting embodiments, the presently disclosed subject matter provides an immunoresponsive cell comprising an antigen-recognizing receptor comprising an extracellular antigen-binding domain, a transmembrane domain, and an intracellular signaling domain. In certain embodiments, the extracellular antigen-binding domain specifically binds to B7-H3 and comprises: (a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; or (b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof.

In certain embodiments, the extracellular antigen-binding domain comprises:
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; or
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof.

In certain embodiments, the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; or
(b) a heavy chain variable region comprising a CDR1 comprising the amino acid sequence set forth in SEQ ID NO: 11 or a conservative modification thereof, a CDR2 comprising an amino acid sequence set forth in SEQ ID NO: 12 or a conservative modification thereof, and a CDR3 comprising the amino acid sequence set forth in SEQ ID NO: 13 or a conservative modification thereof; and a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof.

In certain embodiments, the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular antigen-binding domain comprises a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain is a single-chain variable fragment (scFv). In certain embodiments, the extracellular antigen-binding domain is a human scFv. In certain embodiments, the extracellular antigen-binding domain is a Fab, which is optionally crosslinked. In certain embodiments, the extracellular antigen-binding domain is an F(ab)2.

In certain embodiments, the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7 or SEQ ID NO: 17. In certain embodiments, the extracellular antigen-binding domain comprises a heavy chain variable region comprising the amino acid sequence set forth in SEQ ID NO: 7 or SEQ ID NO: 17.

In certain embodiments, the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or SEQ ID NO: 18. In certain embodiments, the extracellular antigen-binding domain comprises a light chain variable region comprising the amino acid sequence set forth in SEQ ID NO: 8 or SEQ ID NO: 18.

In certain embodiments, extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7 or SEQ ID NO: 17; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or SEQ ID NO: 18. In certain embodiments, the extracellular antigen-binding domain comprises: (a) a heavy chain variable region comprising the amino acid sequence set forth in SEQ ID NO: 7 or SEQ ID NO: 17; and (b) a light chain variable region comprising the amino acid sequence set forth in SEQ ID NO: 8 or SEQ ID NO: 18. In certain embodiments, the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8; or
(b) a heavy chain variable region comprising the amino acid sequence set forth in SEQ ID NO: 17, and a light chain variable region comprising the amino acid sequence set forth in SEQ ID NO: 18.

In certain embodiments, the extracellular antigen-binding domain comprises a linker between a heavy chain variable region and a light chain variable region of the extracellular antigen-binding domain. In certain embodiments, the linker comprises or consists of the amino acid sequence set forth in SEQ ID NO: 31, SEQ ID NO: 32, SEQ ID NO: 33, SEQ ID NO: 34, SEQ ID NO: 35, or SEQ ID NO: 36. In certain embodiments, the extracellular antigen-binding domain comprises a signal peptide that is covalently joined to the 5′ terminus of the extracellular antigen-binding domain.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

In certain embodiments, the intracellular signaling domain comprises a CD3ζ polypeptide. In certain embodiments, the CD3ζ polypeptide comprises or consists of the amino acid sequence set forth in SEQ ID NO: 43.

In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain embodiments, the CD28 polypeptide comprises or consists of amino acids 180 to 220 of SEQ ID NO: 40. In certain embodiments, the CD28 polypeptide comprises a mutated YMNM motif. In certain embodiments, the CD28 polypeptide comprises or consists of the amino acid sequence set forth in SEQ ID NO: 96, SEQ ID NO: 97, SEQ ID NO: 98, SEQ ID NO: 99, or SEQ ID NO: 100. In certain embodiments, the at least one co-stimulatory signaling region comprises a 4-1BB polypeptide. In certain embodiments, the 4-1BB polypeptide comprises or consists of amino acids 214 to 255 of SEQ ID NO: 101.

In certain embodiments, the antigen-recognizing receptor is a chimeric antigen receptor (CAR), or a T-cell like fusion protein. In certain embodiments, the antigen-recognizing receptor is a CAR. In certain embodiments, the CAR comprises the amino acid sequence set forth in SEQ ID NO: 102, SEQ ID NO: 104, or SEQ ID NO: 106. In certain embodiments, the CAR comprises the amino acid sequence set forth in SEQ ID NO: 102.

In certain embodiments, the antigen-recognizing receptor is recombinantly expressed. In certain embodiments, the antigen-recognizing receptor is expressed from a vector. In certain embodiments, the vector is a γ-retroviral vector. In certain embodiments, the antigen-recognizing receptor is constitutively expressed on the surface of the cell.

In certain embodiments, the immunoresponsive cell further comprises a soluble scFv. In certain embodiments, the soluble scFv comprises a heavy chain variable region comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof. In certain embodiments, the soluble scFv comprises a light chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof.

In certain embodiments, the soluble scFv comprises a heavy chain variable region comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof; and a light chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. In certain embodiments, the soluble scFv comprises a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the soluble scFv comprises a heavy chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the soluble scFv comprises a light chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the soluble scFv comprises (a) a heavy chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27; and (b) a light chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the cell comprises a polypeptide comprising an amino acid sequence that is at least about 85%, about 90%, about 95%, about 96%, about 97%, about 98%, or about 99% to the amino acid sequence set forth in set forth in SEQ ID NO: 112. In certain embodiments, the cell comprises a polypeptide comprising the amino acid sequence set forth in set forth in SEQ ID NO: 112.

In certain embodiments, the cell is a cell of the lymphoid lineage or a cell of the myeloid lineage. In certain embodiments, the cell is selected from the group consisting of a T cell, a Natural Killer (NK) cell, and a stem cell from which a lymphoid cell may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell. In certain embodiments, the cell is an NK cell.

In certain non-limiting embodiments, the presently disclosed subject matter provides compositions comprising the cells disclosed herein. In certain embodiments, the composition is a pharmaceutical composition further comprising a pharmaceutically acceptable carrier.

In certain non-limiting embodiments, the presently disclosed subject matter provides a nucleic acid encoding: (a) an antigen-recognizing receptor comprising an extracellular antigen-binding domain, a transmembrane domain, and an intracellular signaling domain, wherein the extracellular antigen-binding domain specifically binds to B7-H3 and comprises: (i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or (ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and (b) a soluble scFv comprising a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26. In certain embodiments, the nucleic acid encodes the amino acid sequence set forth in SEQ ID NO: 112.

In certain non-limiting embodiments, the presently disclosed subject matter provides vectors and lipid nanoparticles comprising the nucleic acids disclosed herein. In certain non-limiting embodiments, the presently disclosed subject matter provides methods of producing an immunoresponsive cell disclosed herein.

In certain non-limiting embodiments, the presently disclosed subject matter provides methods of treating or ameliorating a disease or disorder in a subject, comprising administering to the subject the presently disclosed cells or compositions. In certain embodiments, the disease or disorder is a tumor. In certain embodiments, the tumor is cancer. In certain embodiments, the disease or disorder is selected from the group consisting of osteosarcoma, rhabdomyosarcoma, Ewing's sarcoma, neuroblastoma, desmoplastic small round cell tumor, synovial sarcoma, undifferentiated sarcoma, adrenocortical carcinoma, hepatoblastoma, Wilms tumor, rhabdoid tumor, high-grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma, head and neck cancers, leiomyosarcoma, and melanoma. In certain embodiments, the tumor is high-grade glioma (glioblastoma multiforme). In certain embodiments, the tumor is melanoma. In certain embodiments, the subject is a human.

In certain non-limiting embodiments, the presently disclosed subject matter provides a kit for treating or ameliorating a disease or disorder in a subject, comprising the cell or the composition disclosed herein. In certain embodiments, the kit further comprises written instructions for using the cell or composition for treating or ameliorating a disease or disorder in a subject.

### BRIEF DESCRIPTION OF THE FIGURES

The following Detailed Description, given by way of example, but not intended to limit the invention to specific embodiments described, may be understood in conjunction with the accompanying drawings.

FIG. 1 shows flow cytometry depicting B7-H3 expression on glioma cell lines.

FIGS. 2A-2C depict characterization of B7-H3 expression by established and primary GBM cell lines. FIG. 2A shows flow cytometry depicting B7-H3 expression on 6 independently derived primary GBM cell lines and antigen quantification across the lines depicted in the top graph, surface molecules of B7-H3 per cell. FIGS. 2B and 2C show quantification of antigen density as determined by QuantiBrite assay.

FIGS. 3A-3F show the modulation of B7-H3 in GBM cell lines. FIGS. 3A and 3D depicts the schemes of in vitro experiments. FIGS. 3B and 3E depicts FACS analysis of B7-H3 expression in the tested models. FIGS. 3C and 3F depicts the antigen density of B7-H3 in the tested models.

FIG. 4 shows a representative schematic of the vectors encoding the B7-H3 targeted CARs disclosed herein.

FIG. 5 shows expression of B7-H3 targeting CAR vectors in primary human T cells transduction.

FIG. 6 shows representative transduction of B7-H3 targeting CAR T cells.

FIGS. 7A-7C show 24 hr luciferase-based in vitro killing assay of CAR T cells at varying E:T rations. FIG. 7A depicts the effects on U87MG cell line. FIG. 7B depicts the effects on U251 cell line. [B7-H3-01]28z corresponds to MGA27128z. [B7-H3-01]BBz corresponds to MGA271BBz. [B7-H3-02]28z corresponds to M3028z. [B7-H3-02]BBz corresponds to M30BBz. FIG. 7C shows B7-H3 targeting CAR T cells had robust in vitro cytolytic abilities against primary GBM cell lines. B7-H3 targeting CAR T cells expressing GBM28ζ were cocultured with primary GBM cell lines and demonstrated efficient lysis of tumor cells. CARs demonstrated similar efficacy under each condition (cell line). Data shown is of mean+/−s.e.m. of three independent human donors. Statistical significance was evaluated by a two-way ANOVA, (ns p>0.05, *p<0.05, **p<0.01, ***p<0.001 ****p<0.0001).

FIG. 8 shows 24 hr in vitro cytokine production assay as measured by Luminex assay. [B7-H3-01]28z corresponds to MGA27128z. [B7-H3-01]BBz corresponds to MGA271BBz. [B7-H3-02]28z corresponds to M3028z. [B7-H3-02]BBz corresponds to M30BBz FIGS. 9A-9J show the effects of T cells expressing the B7-H3 targeted CARs disclosed herein in an orthotopic model of GBM. FIG. 9A depicts experimental settings. FIG. 9B depicts a schematic of the GBM-targeting CAR. FIG. 9C depicts the 100d survival curves. FIG. 9D depicts bioluminescent imaging of mice treated with differential interventions. FIG. 9E depicts analysis of bioluminescent imaging of mice treated with differential interventions. FIG. 9F depicts median survival curves of differentially treated groups, Km curve of mice treated with differential doses of B7-H3 targeting CAR T cells, and statistical analysis. FIG. 9G depicts the total weight and relative weight change of mice treated with different doses of the B7-H3 targeted CAR T cells disclosed herein. FIG. 9H shows that despite being administered as little as one log-fold fewer cells, GBM28ζ treated mice respond to therapy at all investigated levels. Data shown is of mean+/−s.e.m. of three independent human donors. FIG. 9I shows responses are also observed through a dose-dependent survival response, and statistical significance was determined through a Log-rank (Mantel-Cox) test evaluating the GBM28ζ treated cohorts against the nontreated cohort (****p<0.0001 for all dose levels). FIG. 9J shows representative bioluminescent imaging of 9H and 9I. FIG. 9K shows statistical comparison of the various treatment cohorts, as determined through a Log-rank (Mantel-Cox) test. (ns p>0.05, *p<0.05, **p<0.01, ***p<0.001, ****p<0.0001). FIG. 9L shows median survival of 9H and 9I. FIG. 9M shows that GBM28ζ does not appear to cause toxicities in NSG mice as determined by changes in mouse weight. Statistical significance at day +27 was determined through a one-way ANOVA evaluating the GBM28ζ treated cohorts against the indicated cohort (ns p>0.05, *p<0.05).

FIGS. 10A-10F show the trafficking of the B7-H3 targeted CAR T cells disclosed herein in intracranial disease sites in an orthotopic model of GBM. FIG. 10A depicts the experimental design. FIG. 10B depicts representative bioluminescence imaging over 14D following ACT to demonstrate trafficking of CAR T cells to disease site. FIG. 10C shows imaging quantification schema. FIG. 10D shows bioluminescence signals increased in the head over time following ACT. Bioluminescence signals remain stable in the body over time following ACT indicating that increase in signal as depicted in FIG. 15B is not due to migration of CAR T cells from the periphery to the disease site. FIG. 10E depicts representative bioluminescent images of T cells and tumor burden at a matched timepoint. FIG. 10F depicts CAR T cells persisted despite reduction in tumor burden.

FIGS. 11A-11F show GBM tumor cells upregulate PD-L1 following in vitro exposure to activated T cell milieu. FIGS. 11A and 11D show schematic of experimental setup. FIG. 11B depicts the FACS analysis of B7-H3 expression in the tested models. FIG. 11C depicts the antigen density of B7-H3 in the tested models. FIG. 11E shows PD-L1 staining on GBM cell lines after 72 hours of treatment. Data shown is of three independent human donors. FIG. 11F shows quantification of PD-L1 expression by MFI after 72 hours of treatment. Data shown is of mean+/−s.e.m. of three independent human donors. Statistical significance was evaluated through an unpaired T test, (*p<0.05, **p=0.0014, ns p=0.077) FIGS. 12A-12H show the effects of CAR T cells on the expression of PD-L1 in GBM models. FIG. 12A shows experimental conditions; subcutaneous tumor xenografts are harvested form mice treated with CAR T cells, 10 day post ACT. FIG. 12B shows representative IHC demonstrate upregulation of PD-L1 on tumor cells from nontreated mice, or mice treated with non-targeting CAR T cells OR B7-H3 targeting CAR T cells. FIG. 12C shows hCD45 staining demonstrated infiltration of GBM28ζ CAR T cells throughout the tumor mass, hCD8 staining demonstrated infiltration of hCD8+ T cells throughout the tumor mass, and PD-L1 strongly upregulated in response to GBM28ζ CAR T cell therapy, overlaying well with the pattern of T cell infiltration. FIGS. 12D and 12E depict GBM infiltrating CAR T cells demonstrate upregulated PD-1; representative flow cytometry demonstrating GBM infiltrating CAR T cells upregulated PD-1.

FIG. 12F shows that NSG mice were subcutaneously implanted with 1×106 ffLuc-tagged U251 cells, treated with a single intravenous administration of 1×106 GBM28ζ CAR T cells after 14 days; tumors and spleens were then harvested 21 days after therapy. FIG. 12G shows representative flow cytometry plot demonstrating increased PD-1 surface staining on intratumoral CAR T cells as compared to peripheral CAR T cells recovered from the spleen. FIG. 12H shows quantification of PD-1 induction by MFI. Data shown is of mean+/−s.e.m. of three independent human donors. Statistical significance was evaluated through an unpaired T test, (***p<0.001).

FIGS. 13A-13C show in vitro characterization of B7-H3 targeted CAR T cells expressing a soluble scFv that binds to PD-1 (armored CAR). FIG. 13A shows a schematic of the vector. FIG. 13B depicts expression of armored CAR in primary human T cells. FIG. 13C depicts incorporation of PD-1 blocking scFv E27 does not augment in vitro cytolytic activity.

FIGS. 14A and 14B show the detection of soluble scFv that binds to PD-1 in armored CAR. FIG. 14A depicts the experimental design. FIG. 14B depicts representative western blotting indicating the expression of the E27 scFv in different T cells.

FIGS. 15A-15C show the effects of armored CAR in a xenograft model. FIG. 15A depicts the experimental design. FIG. 15B depicts tumor volume and survival curve in mice treated with armored CAR with or without additional administration with anti-PD-1 antibody. FIG. 15C depicts the survival curve.

FIGS. 16A-16C show the effects of armored CAR in an orthotopic model of GBM. FIG. 16A depicts experimental settings. FIG. 16B depicts representative bioluminescent images demonstrate that incorporation of PD-1 blocking scFv E27 causes a deeper and longer remission. FIG. 16C depicts imaging mice treated with different doses of the B7-H3 targeted CAR T cells disclosed herein.

FIGS. 17A-17E show PD-1 blockade caused a reduction in exhausted phenotype among tumor infiltrating CAR T cells. FIG. 17A depicts experimental settings. FIG. 17B depicts PD-1 blockade did not cause a change in exhausted phenotype of peripheral CAR T cells. FIGS. 17C and 17D depict intratumoral CAR T cells were less exhausted as determined by PD-1 MFI on tumor infiltrating CAR T cells in the context of concomitant CAR T cell and PD-1 checkpoint blockade. FIG. 17E depicts PD-1 blockade augmented CAR T cell function through an increase in effector cell populations among tumor infiltrating CAR T cells.

FIGS. 18A-18F show representative nucleic acids and vectors disclosed herein.

FIG. 19 shows B7-H3 targeting CAR T cells with variable in vitro cytokine release against GBM cell lines. B7-H3 targeting CAR T cells expressing CD28 or 4-1BB derived CARs were cocultured with B7-H3+ NIH 3T3, CD19+ NIH 3T3, or U251 GBM cells and showed differential production of proinflammatory cytokines. [B7-H3-02]28ζ demonstrated the greatest fold change of the measured cytokines IFNγ, TNFα, GM-CSF, IL-2. Data shown is of mean+/−s.e.m. of three independent human donors. Statistical significance was evaluated by a two-way ANOVA evaluating [B7-H3-02]28ζ against other CARs under identical conditions (*p≤0.05 for the conditions [B7-H3-02]28ζ vs [B7-H3-02]BBζ).

FIGS. 20A and 20B illustrate GMB28ζ-E27 CAR T cells in vitro functionality. FIG. 20A: 1×10⁶ GMB28ζ-E27, GBM28ζ, or corresponding CD19 targeting CAR T cells were stimulated through their CAR for 3 days and supernatant was collected and analyzed by western blot. Clear staining of HA as a marker for E27 was observed at baseline of media originating from nonstimulated E27 CAR T cells, and staining was markedly enriched under stimulation conditions. FIG. 20B: Incorporation of PD-1 blocking scFv E27 does not alter GBM28ζ in vitro cytolytic capacity. Data shown is of mean+/−s.e.m. of three independent healthy donors. Statistical significance was evaluated by a one way ANOVA test, (p>0.05).

### DETAILED DESCRIPTION OF THE INVENTION

Glioblastoma (GBM) is one of the most common and aggressive malignant tumors of the central nervous system (CNS). Current standard of care (SOC) therapy aims at increasing patient life expectancy and focuses on maximal and safe surgical resection combined with radiotherapy (RT) and adjuvant chemotherapy (e.g., temozolomide). Despite this treatment, the median survival of patients diagnosed with GBM is approximately 15 months, with a 2-year life expectancy of less than 30%. For patients with unresectable GBM (up to 35-40% of patients), the prognosis is even poorer (Bausart et al., J Exp Clin Cancer Res 41, 35 (2022)). Recently developed immunotherapies (e.g., adoptive cell therapies and immune-checkpoint blockade) are still ineffective in GBM. Indeed, there is no approved immunotherapy for GBM and many clinical trials have failed.

The presently disclosed subject matter is based, in part, on the discovery that the expression of B7-H3 is upregulated in GBM cells compared to healthy brain tissue. In addition, the inventors of the presently disclosed subject matter observed that GBM cells can develop adaptive resistance against cell therapy by increasing the expression of PD-L1 and inducing upregulation of PD-1 in immune cells.

To date, multiple clinical trials employing CAR T cells targeting disparate antigens in the context of GBM have been completed. Although safety and tolerability of this therapeutic modality were demonstrated, therapeutic efficacy observed in these trials was limited. Subsequent analysis of data accrued from these trials identified the upregulation of T cell inhibitory pathways by tumor cells in response to CAR T cell therapy as a major hurdle. Multiple pathways impede antitumor efficacy of CAR T cells through abrogation of effector functions—including expansion, persistence, and cytolytic activity. However, there have been no reports of successful attempt to treat GBM with immune checkpoint blockade. Combination trials of chemotherapies and/or radiotherapy with immune checkpoint inhibitors have failed to deliver clinical responses as compared to control arms. Moreover GBM does not have similar molecular signatures to those tumors which respond to immune checkpoint blockade, namely a high tumor mutational burden and/or high interferon signatures. Furthermore current attempts to combine CAR T cell therapy with PD-1 inhibitors have yet demonstrated success in the clinic thus providing an opportunity for novel combinations of therapeutic agents (Ahmed et al. (2017). JAMA Oncology 3(8): 1094-1101; Blumenthal et al. (2016). Journal of Neuro-Oncology 129(3): 453-460; Brown et al. (2016). New England Journal of Medicine 375(26): 2561-2569; Brown et al. (2015). Clinical Cancer Research 21(18): 4062-4072; Cristescu et al. (2018). Science 362(6411); Duerinck et al. (2021). J Immunother Cancer 9(6); Goff et al. (2019). Journal of Immunotherapy 42(4): 126-135; Omuro et al. (2018). Neuro Oncol 20(5): 674-686; O'Rourke et al. (2017). Science Translational Medicine 9(399); Reardon et al. (2020). JAMA Oncol 6(7): 1003-1010; Sampson et al. (2020). Nat Rev Cancer 20(1): 12-25; and Yin et al. (2018). Molecular Therapy—Oncolytics 11: 20-38).

The presently disclosed subject matter provides antigen-recognizing receptors (e.g., chimeric antigen receptors (CARs)) that specifically target B7-H3. The presently disclosed subject matter further provides cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and or ameliorating a disease or disorder associated with B7-H3.

Non-limiting embodiments of the present disclosure are described by the present specification and Examples.

For purposes of clarity of disclosure and not by way of limitation, the detailed description is divided into the following subsections:
1. Definitions;
2. B7-H3;
3. Antigen-Recognizing Receptors;
4. Cells;
5. Compositions and Vectors;
6. Polypeptides;
7. Formulations and Administration;
8. Methods of Treatment;
9. Kits; and
10. Exemplary Embodiments.

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., B7-H3). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat numbering system.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 31, which is provided below:

[SEQ ID NO: 31]
SGGGGSGGGSGGGGS

In certain embodiments, the linker comprise or consists of the amino acid sequence set forth in SEQ ID NO: 32, which is provided below:

[SEQ ID NO: 32]
GGGGSGGGGSGGGGS

In certain embodiments, the linker comprises or consists of the amino acid sequence set forth in SEQ ID NO: 33, which is provided below:

[SEQ ID NO: 33]
GGGGSGGGGSGGGGSGGGSGGGGS

In certain embodiments, the linker comprises or consists of the amino acid sequence set forth in SEQ ID NO: 34, which is provided below:

[SEQ ID NO: 34]
GGGGSGGGGSGGGGSGGGGSGGGSGGGGS

In certain embodiments, the linker comprises or consists of the amino acid sequence set forth in SEQ ID NO: 35, which is provided below:

[SEQ ID NO: 35]
GGGGS

In certain embodiments, the linker comprises or consists of the amino acid sequence set forth in SEQ ID NO: 36, which is provided below:

[SEQ ID NO: 36]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife eta., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chem (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain.

In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e−3 and e−100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “a conservative sequence modification” refers to an amino acid modification that does not significantly affect or alter the binding characteristics of the presently disclosed B7-H3-targeted CAR (e.g., the extracellular antigen-binding domain) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “recognize” is meant selectively binds to a target. A T cell that recognizes a tumor can expresses a receptor (e.g., a CAR) that binds to a tumor antigen.

By “signal sequence” or “leader sequence” is meant a peptide sequence (e.g., 5, 10, 15, 20, 25 or 30 amino acids) present at the N-terminus of newly synthesized proteins that directs their entry to the secretory pathway By “specifically binds” or “specifically binds to” or “specifically target” is meant a polypeptide or a fragment thereof that recognizes and/or binds to a biological molecule of interest (e.g., a polypeptide, e.g., a B7-H3 polypeptide), but which does not substantially recognize and/or bind other molecules in a sample, for example, a biological sample, which naturally includes a presently disclosed polypeptide (e.g., a B7-H3 polypeptide).

As used herein, the term “derivative” refers to a compound that is derived from some other compound and maintains its general structure. For example, but without any limitation, trichloromethane (chloroform) is a derivative of methane.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter. 2. B7-H3 B7 homolog 3 protein (B7-H3), also known as CD276, is an immune checkpoint molecule and a costimulatory/coinhibitory immunoregulatory protein that plays a role in the regulation immune system. Initially cloned in 2001 from a cDNA library that was derived from human dendritic cells (DCs), the human B7-H3 gene is located on chromosome 15. The human B7-H3 protein is physiologically expressed as a transmembrane or soluble isoform. The transmembrane B7-H3 is a type I transmembrane protein that comprises an extracellular domain, a transmembrane domain, and a short intracellular domain (Zhou and Jin, Front Immunol. 2021 Jul. 19; 12:701006.). The extracellular domain in murine B7-H3 (2IgB7-H3, B7-H3 VC) is composed of a single pair of immunoglobulin variable domain and constant domain and human B7-H3 (4IgB7-H3, B7-H3 VCVC) is composed of two pairs due to exon duplication. Soluble B7-H3 (sB7-H3), which is cleaved from the surface by a matrix metallopeptidase (MMP) or produced through the alternative splicing of the intron, has also been detected in human sera. In certain embodiments, the antigen-recognizing receptor binds to human B7-H3. In certain embodiments, the human B7-H3 comprises or consists of the amino acid sequence with a UniProt Reference No: Q5ZPR3 (SEQ ID NO: 37) or a fragment thereof. SEQ ID NO: 215 is provided below. In certain embodiments, the B7-H3 comprises an extracellular domain, a transmembrane domain, and a cytoplasmic domain. In certain embodiments, the extracellular domain comprises or consists of amino acids 29 to 466 of SEQ ID NO: 37. In certain embodiments, the transmembrane domain comprises or consists of amino acids 467 to 487 of SEQ ID NO: 37. In certain embodiments, the cytoplasmic domain comprises or consists of amino acids 488 to 534 of SEQ ID NO: 37.

In certain embodiments, the extracellular domain of B7-H3 comprises an Ig-like V-type 1 domain, an Ig-like C2-type 1 domain, an Ig-like V-type 2 domain, and an Ig-like C2-type 2 domain, and EGF-like 5 domain, and an EGF-like 6 domain. In certain embodiments, the Ig-like V-type 1 domain comprises or consists of amino acids 29 to 139 of SEQ ID NO: 37. In certain embodiments, the Ig-like C2-type 1 domain comprises or consists of amino acids 145 to 238 of SEQ ID NO: 37. In certain embodiments, the Ig-like V-type 2 domain comprises or consists of amino acids 243 to 357 of SEQ ID NO: 37. In certain embodiments, the Ig-like C2-type 2 domain comprises or consists of amino acids 363 to 456 of SEQ ID NO: 37.

[SEQ ID NO: 37]
MLRRRGSPGMGVHVGAALGALWFCLTGALEVQVPEDPVVALVGTDATLCCSFSPEPGFSLAQLNLIWQLTDTK
QLVHSFAEGQDQGSAYANRTALFPDLLAQGNASLRLQRVRVADEGSFTCFVSIRDFGSAAVSLQVAAPYSKPS
MTLEPNKDLRPGDTVTITCSSYQGYPEAEVFWQDGQGVPLTGNVTTSQMANEQGLFDVHSILRVVLGANGTYS
CLVRNPVLQQDAHSSVTITPQRSPTGAVEVQVPEDPVVALVGTDATLRCSFSPEPGFSLAQLNLIWQLTDTKQ
LVHSFTEGRDQGSAYANRTALFPDLLAQGNASLRLQRVRVADEGSFTCFVSIRDFGSAAVSLQVAAPYSKPSM
TLEPNKDLRPGDTVTITCSSYRGYPEAEVFWQDGQGVPLTGNVTTSQMANEQGLFDVHSVLRVVLGANGTYSC
LVRNPVLQQDAHGSVTITGQPMTFPPEALWVTVGLSVCLIALLVALAFVCWRKIKQSCEEENAGAEDQDGEGE
GSKTALQPLKHSDSKEDDGQEIA

In certain embodiments, the B7-H3 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 37 or a fragment thereof.

In certain embodiments, the antigen-recognizing receptor binds to a portion of human B7-H3. In certain embodiments, the antigen-recognizing receptor binds to the extracellular domain of B7-H3. In certain embodiments, the antigen-recognizing receptor binds to amino acids 29 to 466 of SEQ ID NO: 37.

In certain embodiments, the antigen-recognizing receptor binds to the Ig-like V-type 1 domain of B7-H3. In certain embodiments, the antigen-recognizing receptor binds to amino acids 29 to 139 of SEQ ID NO: 37. In certain embodiments, the antigen-recognizing receptor binds to the Ig-like C2-type 1 domain of B7-H3. In certain embodiments, the antigen-recognizing receptor binds to amino acids 145 to 238 of SEQ ID NO: 37. In certain embodiments, the antigen-recognizing receptor binds to the Ig-like V-type 2 domain of B7-H3. In certain embodiments, the antigen-recognizing receptor binds to amino acids 243 to 357 of SEQ ID NO: 37. In certain embodiments, the antigen-recognizing receptor binds to Ig-like C2-type 2 domain of B7-H3. In certain embodiments, the antigen-recognizing receptor binds to amino acids 363 to 456 of SEQ ID NO: 37.

### 3. Antigen-Recognizing Receptors

The presently disclosed antigen-recognizing receptors specifically target or bind to B7-H3. In certain embodiments, the antigen-recognizing receptor is a chimeric antigen receptor (CAR). In certain embodiments, the antigen-recognizing receptor is a TCR-like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a B7-H3-targeted antigen-recognizing receptor disclosed herein.

### 3.1. Extracellular Antigen-Binding Domains

In certain embodiments, the extracellular antigen-binding domain of the antigen-recognizing receptor binds to B7-H3.

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is an F(ab)₂.

Any of the foregoing molecules may be comprised in a fusion protein with a heterologous sequence to form the extracellular antigen-binding domain. In certain embodiments, the extracellular antigen-binding domain (embodied, for example, an scFv) binds to B7-H3 (e.g., human B7-H3) with a binding affinity, for example with a dissociation constant (K_{D}) of about 1×10⁻⁸ M or less, about 5×10⁻⁹ M or less, about 1×10⁻⁹ M or less, about 5×10⁻¹ M or less, about 1×10⁻¹ M or less, about 5×10⁻¹¹ M or less, about 1×10⁻¹¹ M or less, about 5×10⁻¹² M or less, or about 1×10⁻¹² M or less.

In certain embodiments, the extracellular antigen-binding domain (embodied, for example, an scFv) binds to B7-H3 (e.g., human B7-H3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹¹ M or less, about 5×10⁻¹¹ M or less, or about 1×10⁻¹¹ M or less, about 5×10⁻¹² M or less, or about 1×10⁻¹² M or less. In certain embodiments, the extracellular antigen-binding domain (embodied, for example, an scFv) binds to B7-H3 (e.g., human B7-H3) with a binding affinity, for example with a dissociation constant (K_{D}) of about 5×10⁻⁹ M or less. In certain embodiments, the extracellular antigen-binding domain (embodied, for example, an scFv) binds to B7-H3 (e.g., human B7-H3) with a binding affinity, for example with a dissociation constant (K_{D}) of about 1×10⁻⁹ M or less. In certain embodiments, the extracellular antigen-binding domain (embodied, for example, an scFv) binds to B7-H3 (e.g., human B7-H3) with a binding affinity, for example with a dissociation constant (K_{D}) of about 2×10⁻⁸ M. In certain embodiments, the extracellular antigen-binding domain (embodied, for example, an scFv) binds to B7-H3 (e.g., human B7-H3) with a binding affinity, for example with a dissociation constant (K_{D}) of about 2×10⁻⁹ M.

Binding of the extracellular antigen-binding domain can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody, or a scFv) specific to the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or autoradiography. In certain embodiments, the B7-H3-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the B7-H3-targeted human scFv is labeled with GFP.

In certain embodiments, the CDRs are identified according to the Kabat numbering system.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the extracellular antigen-binding domain (e.g., an scFv) comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 7. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 7. In certain embodiments, the extracellular antigen-binding domain comprises a V_{H} comprising the amino sequence set forth in SEQ ID NO: 7. An exemplary nucleotide sequence encoding the amino acid sequence of SEQ ID NO: 7 is set forth in SEQ ID NO: 9. SEQ ID NOs: 7 and 9 are provided in Table 1 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain comprises a V_{L} comprising the amino sequence set forth in SEQ ID NO: 8. An exemplary nucleotide sequence encoding the amino acid sequence of SEQ ID NO: 8 is set forth in SEQ ID NO: 10. SEQ ID NOs: 8 and 10 are provided in Table 1 below.

In certain embodiments, the extracellular antigen-binding domain (e.g., an scFv) comprises a V_{H} comprising the amino acid sequence set forth in SEQ ID NO: 7, and a V_{L} Comprising the amino acid sequence set forth in SEQ ID NO: 8. In certain embodiments, the extracellular antigen-binding domain is an scFv. In certain embodiments, the scFv is designated as “M30”. In certain embodiments, the V_{H} and V_{L} are linked via a linker. In certain embodiments, the linker comprises the amino acid sequence set forth in SEQ ID NO: 32.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 1

| CDRs | 1 | 2 | 3 |
|---|---|---|---|
| V_{H} | NYVMH | YINPYNDDVKYNEKF | WGYYGSPLYYFDY |
| | [SEQ ID NO: 1] | KG [SEQ ID NO: 2] | [SEQ ID NO: 3] |
| V_{L} | RASSRLIYMH | ATSNLAS | QQWNSNPPT |
| | [SEQ ID NO: 4] | [SEQ ID NO: 5] | [SEQ ID NO: 6] |

| | |
|---|---|
| Full V_{H} | EVQLQQSGPELVKPGASVKMSCKASGYTFTNYVMHWVKQKPGQGLEWIG |
| | YINPYNDDVKYNEKFKGKATQTSDKSSSTAYMELSSLTSEDSAVYYCAR |
| | WGYYGSPLYYFDYWGQGTTLTVSS [SEQ ID NO: 7] |
| Full V_{L} | QIVLSQSPTILSASPGEKVIMTCRASSRLIYMHWYQQKPGSSPKPWIYA |
| | TSNLASGVPARFSGSGSGTSYSLTISRVEAEDAATYYCQQWNSNPPTFG |
| | TGTKLELKR [SEQ ID NO: 8] |
| DNA | GAAGTACAGCTTCAGCAGTCAGGTCCAGAACTGGTCAAGCCAGGAGCAT |
| for | CCGTAAAGATGTCCTGTAAGGCCAGCGGTTACACATTTACGAATTACGT |
| Full V_{H} | CATGCATTGGGTAAAGCAGAAGCCAGGCCAAGGTCTTGAATGGATCGGA |
| | TACATTAACCCATACAATGACGATGTGAAATACAATGAGAAGTTTAAGG |
| | GCAAAGCTACTCAAACGTCAGATAAATCTTCCAGCACAGCTTACATGGA |
| | GCTGTCTTCCCTGACAAGCGAGGATAGCGCAGTTTATTACTGCGCCCGA |
| | TGGGGTTATTATGGGTCACCTCTTTATTATTTTGACTATTGGGGTCAAG |
| | GGACGACCTTGACCGTATCATCA [SEQ ID NO: 9] |
| DNA | CAGATCGTGTTGAGTCAGAGTCCTACCATATTGTCAGCGAGTCCGGGAG |
| for | AGAAGGTAACTATGACCTGTCGGGCTTCTAGCCGGCTGATATATATGCA |
| Full V_{L} | CTGGTACCAACAAAAGCCGGGCTCATCACCTAAACCTTGGATATATGCT |
| | ACAAGTAATCTTGCGTCAGGCGTCCCAGCCCGGTTCTCCGGCTCCGGTT |
| | CAGGGACAAGTTACTCACTGACTATAAGTCGCGTTGAAGCCGAGGATGC |
| | CGCCACGTATTATTGTCAGCAATGGAACAGTAACCCTCCCACATTTGGC |
| | ACGGGGACTAAGCTGGAATTGAAAAGG [SEQ ID NO: 10] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2.

In certain embodiments, the extracellular antigen-binding domain (e.g., an scFv) comprises a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 146or a conservative modification thereof.

In certain embodiments, the extracellular antigen-binding domain (e.g., an scFv) comprises a V_{H} comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and a V_{L} comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 17. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 17. In certain embodiments, the extracellular antigen-binding domain comprises a V_{H} comprising the amino sequence set forth in SEQ ID NO: 17. An exemplary nucleotide sequence encoding the amino acid sequence of SEQ ID NO: 17 is set forth in SEQ ID NO: 19. SEQ ID NO: 17 and 19 are provided in Table 2 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 18. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 18. In certain embodiments, the extracellular antigen-binding domain comprises a V_{L} comprising the amino sequence set forth in SEQ ID NO: 18. An exemplary nucleotide sequence encoding the amino acid sequence of SEQ ID NO: 18 is set forth in SEQ ID NO: 20. SEQ ID NO: 18 and 20 are provided in Table 2 below.

In certain embodiments, the extracellular antigen-binding domain (e.g., an scFv) comprises a V_{H} comprising the amino acid sequence set forth in SEQ ID NO: 17, and a V_{L} comprising the amino acid sequence set forth in SEQ ID NO: 18. In certain embodiments, the extracellular antigen-binding domain is an scFv. In certain embodiments, the scFv is designated as “MGA271”. In certain embodiments, the V_{H} and V_{L} are linked via a linker. In certain embodiments, the linker comprises the amino acid sequence set forth in SEQ ID NO: 32.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 2

| CDRS | 1 | 2 | 3 |
|---|---|---|---|
| V_{H} | SFGMH | YISSDSSAIYYADTV | GRENIYYGSRLDY |
| | [SEQ ID NO: 11] | KG [SEQ ID NO: | [SEQ ID NO: |
| | | 12] | 13] |
| V_{L} | KASQNVDTNVA | SASYRYS | QQYNNYPFT |
| | [SEQ ID NO: 14] | [SEQ ID NO: 15] | [SEQ ID NO: 16] |

| | |
|---|---|
| Full V_{H} | EVQLVESGGGLVQPGGSLRLSCAASGFTFSSFGMHWVRQAPGKGLEW |
| | VAYISSDSSAIYYADTVKGRFTISRDNAKNSLYLQMNSLRDEDTAVY |
| | YCGRGRENIYYGSRLDYWGQGTTVTVSS [SEQ ID NO: 17] |
| Full V_{L} | DIQLTQSPSFLSASVGDRVTITCKASQNVDTNVAWYQQKPGKAPKAL |
| | IYSASYRYSGVPSRFSGSGSGTDFTLTISSLQPEDFATYYCQQYNNY |
| | PFTFGQGTKLEIKR [SEQ ID NO: 18] |
| DNA for | GAGGTCCAGCTTGTTGAATCAGGGGGCGGCCTCGTCCAACCGGGGGG |
| Full V_{H} | CTCACTGCGCTTGTCTTGCGCTGCCTCTGGATTTACCTTTAGTTCTT |
| | TCGGAATGCATTGGGTGCGGCAAGCACCCGGCAAGGGGTTGGAGTGG |
| | GTAGCGTATATAAGCAGCGACTCCTCTGCCATCTACTACGCGGATAC |
| | GGTGAAAGGCAGATTTACGATTAGTAGAGACAACGCTAAGAATTCAT |
| | TGTACTTGCAAATGAACAGCCTCAGAGACGAGGATACCGCCGTGTAC |
| | TATTGTGGAAGGGGCAGGGAGAATATATACTATGGGTCAAGATTGGA |
| | TTATTGGGGACAGGGGACGACAGTCACGGTGAGCAGT [SEQ ID |
| | NO: 19] |
| DNA for | GACATTCAGCTCACTCAGTCACCATCTTTTCTGTCAGCGTCTGTGGG |
| Full V_{L} | GGACAGAGTAACGATAACGTGCAAAGCGAGTCAGAACGTTGATACCA |
| | ATGTGGCCTGGTATCAGCAAAAGCCCGGTAAAGCCCCGAAGGCGCTG |
| | ATCTATTCTGCCAGCTACCGATACTCAGGCGTTCCTTCACGCTTTAG |
| | CGGGTCAGGCTCTGGTACCGACTTCACTCTTACGATCAGTTCATTGC |
| | AGCCCGAAGACTTCGCCACTTACTACTGCCAACAATATAATAACTAC |
| | CCTTTCACGTTTGGCCAAGGCACCAAGTTGGAGATTAAGCGG [SEQ |
| | ID NO: 20] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 7, SEQ ID NO: 8, SEQ ID NO: 17, or SEQ ID NO: 18) may contain substitutions (e.g., conservative substitutions), insertions, or deletions relative to the specified sequence(s), but retain the ability to bind to B7-H3.

In certain embodiments, a total of 1 to 10 amino acids are substituted, inserted, and/or deleted in a specific sequence (e.g., SEQ ID NO: 7, SEQ ID NO: 8, SEQ ID NO: 17, or SEQ ID NO: 18). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 7, 8, 17, or 18 including post-translational modifications of that sequence (SEQ ID NO: 7, 8, 17, or 18).

In certain embodiments, the extracellular antigen-binding domain of a presently disclosed CAR cross-competes for binding to B7-H3 (e.g., human B7-H3) with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g.,). In certain embodiments, the extracellular antigen-binding domain of a presently disclosed CAR cross-competes for binding to B7-H3 (e.g., human B7-H3) with a reference antibody or an antigen-binding portion thereof comprising the V_{H} and V_{L} sequences of, for example, any one of the presently disclosed scFvs (e.g., M30 and MGA271).

In certain embodiments, the extracellular antigen-binding domain of a presently disclosed CAR cross-competes for binding to B7-H3 (e.g., human B7-H3) with a reference antibody or an antigen-binding portion thereof comprising the V_{H} CDR1, CDR2, and CDR3 sequences and the V_{L} CDR1, CDR2, and CDR3 sequences of scFv M30. For example, the extracellular antigen-binding domain of a presently disclosed CAR cross-competes for binding to B7-H3 (e.g., human B7-H3) with a reference antibody or an antigen-binding portion thereof comprising a V_{H} CDR1 comprising the amino acid sequence set forth in SEQ ID NO: 1, a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising amino acids having the sequence set forth in SEQ ID NO: 5; and a V_{L} CDR3 comprising amino acids having the sequence set forth in SEQ ID NO: 6. In certain embodiments, the extracellular antigen-binding domain of a presently disclosed CAR cross-competes for binding to B7-H3 (e.g., human B7-H3) with a reference antibody or an antigen-binding portion thereof comprising the V_{H} and V_{L} sequences of scFv M30. For example, the extracellular antigen-binding domain of a presently disclosed CAR cross-competes for binding to B7-H3 (e.g., human B7-H3) with a reference antibody or an antigen-binding portion thereof comprising a V_{H} comprising amino acids having the sequence set forth in SEQ ID NO: 7, and a V_{L} comprising amino acids having the sequence set forth in SEQ ID NO: 8.

In certain embodiments, the extracellular antigen-binding domain binds to the same epitope region on B7-H3 (e.g., human B7-H3) as the reference antibody or antigen-binding portion thereof. For example, the extracellular antigen-binding domain of a presently disclosed CAR binds to the same epitope region on B7-H3 (e.g., human B7-H3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., M30 and MGA271). In certain embodiments, the extracellular antigen-binding domain of a presently disclosed CAR binds to the same epitope region on B7-H3 (e.g., human B7-H3) as a reference antibody or an antigen-binding portion thereof comprising the V_{H} and V_{L} sequences of, for example, any one of the presently disclosed scFvs (e.g., M30 and MGA271).

In certain embodiments, the extracellular antigen-binding domain cross-competes for binding to B7-H3 (e.g., human B7-H3) with a reference antibody or an antigen-binding portion thereof. For example, the extracellular antigen-binding domain of a presently disclosed CAR cross-competes for binding to B7-H3 (e.g., human B7-H3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., M30 and MGA271). In certain embodiments, the extracellular antigen-binding domain of a presently disclosed CAR binds to the same epitope region on B7-H3 (e.g., human B7-H3) as a reference antibody or an antigen-binding portion thereof comprising the V_{H} and V_{L} sequences of, for example, any one of the presently disclosed scFvs (e.g., M30 and MGA271).

Extracellular antigen-binding domains that cross-compete or compete with the reference antibody or antigen-binding portions thereof for binding to B7-H3 (e.g., human B7-H3) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a B7-H3 polypeptide, admixing the reference antibody with a test extracellular antigen-binding domain, measuring a second binding of the reference antibody to the B7-H3 polypeptide in the presence of the test extracellular antigen-binding domain, and comparing the initial binding with the second binding of the reference antibody, wherein a decreased second binding of the reference antibody to the B7-H3 polypeptide in comparison to the initial binding indicates that the test extracellular antigen-binding domain cross-competes with the reference antibody for binding to B7-H3, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the B7-H3 polypeptide is expressed in cells, e.g., in a flow cytometry test. In certain embodiments, the B7-H3 polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to B7-H3) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to a B7-H3 polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to B7-H3. In certain embodiments, the assays are performed at room temperature.

In certain embodiments, the antibody-binding assay comprises measuring an initial binding of a test extracellular antigen-binding domain to a B7-H3 polypeptide, admixing the test extracellular antigen-binding domain with a reference antibody, measuring a second binding of the test extracellular antigen-binding domain to the B7-H3 polypeptide in the presence of the reference antibody, and comparing the initial binding with the second binding of the test extracellular antigen-binding domain, where a decreased second binding of the test extracellular antigen-binding domain to the B7-H3 polypeptide in comparison to the initial binding indicates that the test extracellular antigen-binding domain cross-competes with the reference antibody for binding to B7-H3, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the B7-H3 polypeptide is expressed in cells, e.g., in a flow cytometry test. In certain embodiments, the B7-H3 polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to B7-H3) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to a B7-H3 polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to B7-H3. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises or consists of the amino acid sequence set forth in SEQ ID NO: 31. In certain embodiments, the linker comprises or consists of the amino acid sequence set forth in SEQ ID NO: 32. In certain embodiments, the linker comprises or consists of the amino acid sequence set forth in SEQ ID NO: 33. In certain embodiments, the linker comprises or consists of the amino acid sequence set forth in SEQ ID NO: 34. In certain embodiments, the linker comprises or consists of the amino acid sequence set forth in SEQ ID NO: 35. In certain embodiments, the linker comprises or consists of the amino acid sequence set forth in SEQ ID NO: 36.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

### 3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to B7-H3, a transmembrane domain, and an intracellular signaling domain.

### 3.2.1. Extracellular Antigen-Binding Domain of a CAR

The extracellular antigen-binding domain of the CAR can be any extracellular antigen-binding domain disclosed herein, e.g., in Section 3.1.

In certain embodiments, the CAR comprises an extracellular antigen-binding domain disclosed in Section 3.1.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide.

### 3.2.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99%, or about 100% homologous or identical to the amino acid sequence having an NCBI Reference No: NP_001139345.1 (SEQ ID NO: 38) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 38, which is at least 20, at least 30, at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, 137 to 209, or 200 to 235 of SEQ ID NO: 38. In certain embodiments, the transmembrane domain of the CAR comprises a CD8 polypeptide comprising or consisting of amino acids 137 to 209 of SEQ ID NO: 38. SEQ ID NO: 38 is provided below.

[SEQ ID NO: 38]
MALPVTALLLPLALLLHAARPSQFRVSPLDRTWNLGETVELKCQVLLSNPTSGCSWLFQPRGAAASPTFLLYL
SQNKPKAAEGLDTQRFSGKRLGDTFVLTLSDFRRENEGYYFCSALSNSIMYFSHFVPVFLPAKPTTTPAPRPP
TPAPTIASQPLSLRPEACRPAAGGAVHTRGLDFACDIYIWAPLAGTCGVLLLSLVITLYCNHRNRRRVCKCPR
PVVKSGDKPSLSARYV

In certain embodiments, the transmembrane domain of the CAR comprises a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99%, or about 100% homologous or identical to the amino acid sequence having an NCBI Reference No: AAA92533.1 (SEQ ID NO: 39) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 39, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 39. In certain embodiments, the transmembrane domain of the CAR comprises a CD8 polypeptide comprising or consisting of amino acids 151 to 219 of SEQ ID NO: 39. SEQ ID NO: 39 is provided below.

[SEQ ID NO: 39]
MASPLTRFLSLNLLLMGESIILGSGEAKPQAPELRIFPKKMDAELGQKVDLVCEVLGSVSQGCSWLFQNSSSK
LPQPTFVVYMASSHNKITWDEKLNSSKLFSAVRDTNNKYVLTLNKFSKENEGYYFCSVISNSVMYFSSVVPVL
QKVNSTTTKPVLRTPSPVHPTGTSQPQRPEDCRPRGSVKGTGLDFACDIYIWAPLAGICVAPLLSLIITLICY
HRSRKRVCKCPRPLVRQEGKPRPSEKIV

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99%, or 100% homologous or identical to the amino acid sequence having an NCBI Reference No: NP_006130 (SEQ ID NO: 40) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 40 which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 40. In certain embodiments, the transmembrane domain of the CAR comprises a CD28 polypeptide comprising or consisting of amino acids 153 to 179 of SEQ ID NO: 40. SEQ ID NO: 40 is provided below:

[SEQ ID NO: 40]
MLRLLLALNLFPSIQVTGNKILVKQSPMLVAYDNAVNLSCKYSYNLFSREFRASLHKGLDSAVEVCVVYGNYS
QQLQVYSKTGFNCDGKLGNESVTFYLQNLYVNQTDIYFCKIEVMYPPPYLDNEKSNGTIIHVKGKHLCPSPLF
PGPSKPFWVLVVVGGVLACYSLLVTVAFIIFWVRSKRSRLLHSDYMNMTPRRPGPTRKHYQPYAPPRDFAAYR
S

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99%, or 100% homologous or identical to the amino acid sequence having an NCBI Reference No: NP_031668.3 (SEQ ID No: 41) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 41, which is at least 20, or at least 30, or at least 40, or at least 50, and up to 218 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 41. In certain embodiments, the transmembrane domain of the CAR comprises a CD28 polypeptide comprising or consisting of amino acids 151 to 177 of SEQ ID NO: 41. SEQ ID NO: 41 is provided below:

[SEQ ID NO: 41]
MTLRLLFLALNFFSVQVTENKILVKQSPLLVVDSNEVSLSCRYSYNLLAKEFRASLYKGVNSDVEVCVGNGNF
TYQPQFRSNAEFNCDGDFDNETVTFRLWNLHVNHTDIYFCKIEFMYPPPYLDNERSNGTIIHIKEKHLCHTQS
SPKLFWALVVVAGVLFCYGLLVTVALCVIWTNSRRNRLLQSDYMNMTPRRPGLTRKPYQPYAPARDFAAYRP

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, the CH₂CH₃ region of immunoglobulin and portions of CD3, a portion of a CD28 polypeptide (e.g., a portion of SEQ ID NO: 40 or 41), a portion of a CD8 polypeptide (e.g., a portion of SEQ ID NO: 38 or 39), a variation of any of the foregoing which is at least about 80%, at least about 85%, at least about 90%, at least about 95%, or at least about 100% homologous or identical thereto, or a synthetic spacer sequence.

### 3.2.3. Intracellular Signaling Domain of a CAR

In certain embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), and three functional basic-rich stretch (BRS) regions (BRS1, BRS2, and BRS3). CD3ζ transmits an activation signal to the cell (e.g., a cell of the lymphoid lineage, e.g., a T cell) after the antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having an NCBI Reference No: NP_932170 (SEQ ID NO: 42) or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 42, which is at least 20, or at least 30, or at least 40, or at least 50, and up to 164 amino acids in length. Alternatively or additionally, in non-limiting various embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 42. In certain embodiments, the intracellular signaling domain of the CAR comprises a CD3ζ polypeptide comprising or consisting of amino acids 52 to 164 of SEQ ID NO: 42. SEQ ID NO: 42 is provided below:

[SEQ ID NO: 42]
MKWKALFTAAILQAQLPITEAQSFGLLDPKLCYLLDGILFIYGVILTALFLRVKFSRSADAPAYQQGQNQLYN
ELNLGRREEYDVLDKRRGRDPEMGGKPQRRKNPQEGLYNELQKDKMAEAYSEIGMKGERRRGKGHDGLYQGLS
TATKDTYDALHMQALPPR

In certain embodiments, the intracellular signaling domain of the CAR comprises a CD3 polypeptide comprising or consisting of the amino acid sequence set forth in SEQ ID NO: 43. SEQ ID NO: 43 is provided below.

[SEQ ID NO: 43]
RVKFSRSADAPAYQQGQNQLYNELNLGRREEYDVLDKRRGRDPEMGGKPRRKNPQEGLYNELQKDKMAEAYSE
IGMKGERRRGKGHDGLYQGLSTATKDTYDALHMQALPPR

An exemplary nucleotide sequence encoding the amino acid sequence of SEQ ID NO: 43 is set forth in SEQ ID NO: 44, which is provided below.

[SEQ ID NO: 44]
AGAGTGAAGTTCAGCAGGAGCGCAGACGCCCCCGCGTACCAGCAGGGCCAGAACCAGCTCTATAACGAGCTCA
ATCTAGGACGAAGAGAGGAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the intracellular signaling domain of the CAR comprises a co-stimulatory signaling region that comprises a CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 40 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 40, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 40. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 40.

In certain embodiments, the intracellular signaling domain of the CAR comprises a co-stimulatory signaling region that comprises a CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 41 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 41, which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 41. In certain embodiments, the co-stimulatory signaling region of a presently disclosed CAR comprises a CD28 polypeptide that comprises or consists of the amino acids 178 to 218 of SEQ ID NO: 41.

In certain embodiments, the co-stimulatory signaling region of a presently disclosed CAR comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YMNM motif, and two proline-rick motifs: PRRP motif, and PYAP motif. The CD28 intracellular motifs can serve as docking sites for a number of adaptor molecules that interact with these motifs through their SH2 or SH3 domains.

Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K. The native YMNM motif comprises or consists of the amino acid sequence set forth in SEQ ID NO: 45. The PRRP motif and PYAP motif comprise or consist of the amino acid sequence set forth in SEQ ID NO: 114 and SEQ ID NO: 115, respectively. SEQ ID NO: 45, SEQ ID NO: 114, and SEQ ID NO: 115 are provided below:

[SEQ ID NO: 45]
YMNM
[SEQ ID NO: 114]
PRRP
[SEQ ID NO: 115]
PYAP

In certain embodiments, a native YMNM motif consists of the amino acid sequence set forth in YMNM (SEQ ID NO: 45). In certain embodiments, a native YMNM motif binds to the p85 subunit of PI3K via a consensus sequence YMxM (SEQ ID NO: 46), wherein x is not an aspartic acid (N). In certain embodiments, a native YMNM motif binds to Grb2 and/or GADs via a consensus sequence YxNx (SEQ ID NO: 47), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YMNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif, i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 47), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 48), YSNV (SEQ ID NO: 49), YKNL (SEQ ID NO: 50), YENQ (SEQ ID NO: 51), YKNI (SEQ ID NO: 52), YINQ (SEQ ID NO: 53), YHNK (SEQ ID NO: 54), YVNQ (SEQ ID NO: 55), YLNP (SEQ ID NO: 56), YLNT (SEQ ID NO: 57), YDND (SEQ ID NO: 58), YENI (SEQ ID NO: 59), YENL (SEQ ID NO: 60), YKNQ (SEQ ID NO: 61), YKNV (SEQ ID NO: 62), or YANG (SEQ ID NO: 63). In certain embodiments, the mutated YMNM motif consists of the amino acid sequence set forth in YSNV (SEQ ID NO: 49). In certain embodiments, the mutated YMNM motif consists of the amino acid sequence set forth in YKNI (SEQ ID NO: 52). In certain embodiments, the mutated YMNM motif consists of the amino acid sequence set forth in YENV (SEQ ID NO: 48). In certain embodiments, the mutated YMNM motif consists of the amino acid sequence set forth in YKNL (SEQ ID NO: 50).

In certain embodiments, the mutated YMNM motif is a PI3K-permissive mutant. In certain embodiments, the mutated YMNM motif consists of the amino acid sequence set forth in YMxM (SEQ ID NO: 46), wherein x is not an aspartic acid (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 64), YMPM (SEQ ID NO: 65), YMRM (SEQ ID NO: 66), or YMSM (SEQ ID NO: 67). In certain embodiments, the mutated YMNM motif consists of the amino acid sequence set forth in YMDM (SEQ ID NO: 64).

In certain embodiments, the mutated YMNM motif consists of the amino acid sequence set forth in YbxM (SEQ ID NO: 68), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YTHM (SEQ ID NO: 69), YVLM (SEQ ID NO: 70), YIAM (SEQ ID NO: 71), YVEM (SEQ ID NO: 72), YVKM (SEQ ID NO: 73), or YVPM (SEQ ID NO: 74).

In certain embodiments, the mutated YMNM motif consists of the amino acid sequence set forth in YMxb (SEQ ID NO: 75), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 76).

Certain mutated YMNM motifs are described in Mol Cell Proteomics. 2010 November; 9(11):2391-404; Virology. 2015 May; 0: 568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 77) or YxNM (SEQ ID NO: 78), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 79), YENM (SEQ ID NO: 80), YMNQ (SEQ ID NO: 81), YMNL (SEQ ID NO: 82), or YSNM (SEQ ID NO: 83).

In certain embodiments, the mutated YMNM motif is a non-functional mutant. In certain embodiments, the mutated YMNM motif consists of the amino acid sequence Ybxb (SEQ ID NO: 84), wherein x is not an aspartic acid (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 85), YAAA (SEQ ID NO: 86), YFFF (SEQ ID NO: 87), YETV (SEQ ID NO: 88), YQQQ (SEQ ID NO: 89), YHAE (SEQ ID NO: 90), YLDL (SEQ ID NO: 91), YLIP (SEQ ID NO: 92), YLRV (SEQ ID NO: 93), YTAV (SEQ ID NO: 94), or YVHV (SEQ ID NO: 95). In certain embodiments, the mutated YMNM motif consists of the amino acid sequence set forth in YGGG (SEQ ID NO: 85).

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YENV (SEQ ID NO: 48), wherein the CD28 polypeptide comprises or consists of the amino acid sequence set forth in SEQ ID NO: 96. SEQ ID NO: 96 is provided below.

[SEQ ID NO: 96]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 52), wherein the CD28 polypeptide comprises or consists of the amino acid sequence set forth in SEQ ID NO: 97. SEQ ID NO: 97 is provided below.

[SEQ ID NO: 97]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YMDM (SEQ ID NO: 64), wherein the CD28 polypeptide comprises or consists of the amino acid sequence set forth in SEQ ID NO: 98. SEQ ID NO: 98 is provided below.

[SEQ ID NO: 98]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 85), wherein the CD28 polypeptide comprises or consists of the amino acid sequence set forth in SEQ ID NO: 99. SEQ ID NO: 99 is provided below.

[SEQ ID NO: 99]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 49), wherein the CD28 polypeptide comprises or consists of the amino acid sequence set forth in SEQ ID NO: 100. SEQ ID NO: 100 is provided below.

[SEQ ID NO: 100]
RSKRSRLLHSDYSNVTPRRPGPTRKHYQPYAPPRDFAAYRS

In certain embodiments, the intracellular signaling domain of the presently disclosed CAR comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding CARs including CD28 polypeptide comprising a mutated YMNM motif can be found in International Patent Publication No. WO 2021/158850, which is incorporated by reference in its entirety.

In certain embodiments, the intracellular signaling domain of the CAR comprises a co-stimulatory signaling region that comprises a 4-1BB polypeptide, e.g., an intracellular domain of 4-1BB or a fragment thereof. In certain embodiments, the intracellular signaling domain of the CAR comprises a co-stimulatory signaling region that comprises a 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 101) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 101, which is at least 20, or at least 30, or at least 40, or at least 50, or at least 100, or at least 150, or at least 150, and up to 255 amino acids in length. Alternatively or additionally, in non-limiting various embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 101. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 101. SEQ ID NO: 101 is provided below.

[SEQ ID NO: 101]
MGNSCYNIVATLLLVLNFERTRSLQDPCSNCPAGTFCDNNRNQICSPCPPNSFSSAGGQRTCDICRQCKGVFR
TRKECSSTSNAECDCTPGFHCLGAGCSMCEQDCKQGQELTKKGCKDCCFGTFNDQKRGICRPWTNCSLDGKSV
LVNGTKERDVVCGPSPADLSPGASSVTPPAPAREPGHSPQIISFFLALTSTALLFLLFFLTLRFSVVKRGRKK
LLYIFKQPFMRPVQTTQEEDGCSCRFPEEEEGGCEL

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 3.2.4. Exemplified CARs

In certain embodiments, the CAR is a B7-H3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 40. In certain embodiments, the intracellular signaling domain comprises (i) a CD3ζ polypeptide comprising the amino acid sequence set forth in SEQ ID NO: 43, and (ii) a co-stimulatory signaling region comprising a CD28 polypeptide comprising amino acids 180 to 220 of SEQ ID NO: 40. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is designed as “M3028z”. In certain embodiments, the CAR comprises or consists of the amino acid sequence set forth in SEQ ID NO: 102. SEQ ID NO: 102 is provided below.

[SEQ ID NO: 102]
MALPVTALLLPLALLLHAARPEVQLQQSGPELVKPGASVKMSCKASGYTFTNYVMHWVKQKPGQGLEWIGYIN
PYNDDVKYNEKFKGKATQTSDKSSSTAYMELSSLTSEDSAVYYCARWGYYGSPLYYFDYWGQGTTLTVSSGGG
GSGGGGSGGGGSQIVLSQSPTILSASPGEKVTMTCRASSRLIYMHWYQQKPGSSPKPWIYATSNLASGVPARF
SGSGSGTSYSLTISRVEAEDAATYYCQQWNSNPPTFGTGTKLELKREQKLISEEDLAAAIEVMYPPPYLDNEK
SNGTIIHVKGKHLCPSPLFPGPSKPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR

An exemplary nucleotide sequence encoding the amino acid sequence of SEQ ID NO: 102 is set forth in SEQ ID NO: 103, which is as provided below.

[SEQ ID NO: 103]
ATGGCTCTCCCAGTGACTGCCCTACTGCTTCCCCTAGCGCTTCTCCTGCATGCAgccaggccgGAAGTACAGC
TTCAGCAGTCAGGTCCAGAACTGGTCAAGCCAGGAGCATCCGTAAAGATGTCCTGTAAGGCCAGCGGTTACAC
ATTTACGAATTACGTCATGCATTGGGTAAAGCAGAAGCCAGGCCAAGGTCTTGAATGGATCGGATACATTAAC
CCATACAATGACGATGTGAAATACAATGAGAAGTTTAAGGGCAAAGCTACTCAAACGTCAGATAAATCTTCCA
GCACAGCTTACATGGAGCTGTCTTCCCTGACAAGCGAGGATAGCGCAGTTTATTACTGCGCCCGATGGGGTTA
TTATGGGTCACCTCTTTATTATTTTGACTATTGGGGTCAAGGGACGACCTTGACCGTATCATCAGGTGGAGGT
GGATCAGGTGGAGGTGGATCTGGTGGAGGTGGATCTCAGATCGTGTTGAGTCAGAGTCCTACCATATTGTCAG
CGAGTCCGGGAGAGAAGGTAACTATGACCTGTCGGGCTTCTAGCCGGCTGATATATATGCACTGGTACCAACA
AAAGCCGGGCTCATCACCTAAACCTTGGATATATGCTACAAGTAATCTTGCGTCAGGCGTCCCAGCCCGGTTC
TCCGGCTCCGGTTCAGGGACAAGTTACTCACTGACTATAAGTCGCGTTGAAGCCGAGGATGCCGCCACGTATT
ATTGTCAGCAATGGAACAGTAACCCTCCCACATTTGGCACGGGGACTAAGCTGGAATTGAAAAGGGAACAGAA
ACTGATCTCCGAGGAGGACCTCGCGGCCGCAATTGAAGTTATGTATCCTCCTCCTTACCTAGACAATGAGAAG
AGCAATGGAACCATTATCCATGTGAAAGGGAAACACCTTTGTCCAAGTCCCCTATTTCCCGGACCTTCTAAGC
CCTTTTGGGTGCTGGTGGTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACATGACTCCCCGCCGCCCCGGG
CCCACCCGCAAGCATTACCAGCCCTATGCCCCACCACGCGACTTCGCAGCCTATCGCTCCAGAGTGAAGTTCA
GCAGGAGCGCAGACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTACAGCCACCAAGGACACCTACGA
CGCCCTTCACATGCAGGCCCTGCCCCCTCGCTAA

In certain embodiments, the CAR is a B7-H3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD8 polypeptide (e.g., a transmembrane domain of human C28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD8 polypeptide that comprises amino acids 137 to 209 of SEQ ID NO: 38. In certain embodiments, the intracellular signaling domain comprises (i) a CD3ζ polypeptide comprising the amino acid sequence set forth in SEQ ID NO: 42, and (ii) a co-stimulatory signaling region comprising a 4-1BB polypeptide comprising amino acids 214 to 255 of SEQ ID NO: 101. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is designed as “M30BBz”. In certain embodiments, the CAR comprises or consists of the amino acid sequence set forth in SEQ ID NO: 104. SEQ ID NO: 104 is provided below.

[SEQ ID NO: 104]
MALPVTALLLPLALLLHAARPEVQLQQSGPELVKPGASVKMSCKASGYTFTNYVMHWVKQKPGQGLEWIGYIN
PYNDDVKYNEKFKGKATQTSDKSSSTAYMELSSLTSEDSAVYYCARWGYYGSPLYYFDYWGQGTTLTVSSGGG
GSGGGGSGGGGSQIVLSQSPTILSASPGEKVTMTCRASSRLIYMHWYQQKPGSSPKPWIYATSNLASGVPARF
SGSGSGTSYSLTISRVEAEDAATYYCQQWNSNPPTFGTGTKLELKREQKLISEEDLAAAPTTTPAPRPPTPAP
TIASQPLSLRPEACRPAAGGAVHTRGLDFACDIYIWAPLAGTCGVLLLSLVITLYCNKRGRKKLLYIFKQPFM
RPVQTTQEEDGCSCRFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALPPR

An exemplary nucleotide sequence encoding the amino acid sequence of SEQ ID NO: 104 is set forth in SEQ ID NO: 105, which is as provided below.

[SEQ ID NO: 105]
ATGGCTCTCCCAGTGACTGCCCTACTGCTTCCCCTAGCGCTTCTCCTGCATGCAgccaggccgGAAGTACAGC
TTCAGCAGTCAGGTCCAGAACTGGTCAAGCCAGGAGCATCCGTAAAGATGTCCTGTAAGGCCAGCGGTTACAC
ATTTACGAATTACGTCATGCATTGGGTAAAGCAGAAGCCAGGCCAAGGTCTTGAATGGATCGGATACATTAAC
CCATACAATGACGATGTGAAATACAATGAGAAGTTTAAGGGCAAAGCTACTCAAACGTCAGATAAATCTTCCA
GCACAGCTTACATGGAGCTGTCTTCCCTGACAAGCGAGGATAGCGCAGTTTATTACTGCGCCCGATGGGGTTA
TTATGGGTCACCTCTTTATTATTTTGACTATTGGGGTCAAGGGACGACCTTGACCGTATCATCAGGTGGAGGT
GGATCAGGTGGAGGTGGATCTGGTGGAGGTGGATCTCAGATCGTGTTGAGTCAGAGTCCTACCATATTGTCAG
CGAGTCCGGGAGAGAAGGTAACTATGACCTGTCGGGCTTCTAGCCGGCTGATATATATGCACTGGTACCAACA
AAAGCCGGGCTCATCACCTAAACCTTGGATATATGCTACAAGTAATCTTGCGTCAGGCGTCCCAGCCCGGTTC
TCCGGCTCCGGTTCAGGGACAAGTTACTCACTGACTATAAGTCGCGTTGAAGCCGAGGATGCCGCCACGTATT
ATTGTCAGCAATGGAACAGTAACCCTCCCACATTTGGCACGGGGACTAAGCTGGAATTGAAAAGGGAACAGAA
ACTGATCTCCGAGGAGGACCTCGCGGCCGCACCCACCACGACGCCAGCGCCGCGACCACCAACCCCGGCGCCC
ACGATCGCGTCGCAGCCCCTGTCCCTGCGCCCAGAGGCGTGCCGGCCAGCGGCGGGGGGCGCAGTGCACACGA
GGGGGCTGGACTTCGCCTGTGATATCTACATCTGGGCGCCCCTGGCCGGGACTTGTGGGGTCCTTCTCCTGTC
ACTGGTTATCACCCTTTACTGCAACAAACGGGGCAGAAAGAAGCTCCTGTATATATTCAAACAACCATTTATG
AGACCAGTACAAACTACTCAAGAGGAAGATGGCTGTAGCTGCCGATTTCCAGAAGAAGAAGAAGGAGGATGTG
AACTGAGAGTGAAGTTCAGCAGGAGCGCAGACGCCCCCGCGTACCAGCAGGGCCAGAACCAGCTCTATAACGA
GCTCAATCTAGGACGAAGAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTTTACCAGGGTCTCAGTACAGC
CACCAAGGACACCTACGACGCCCTTCACATGCAGGCCCTGCCCCCTCGCTAA

In certain embodiments, the CAR is a B7-H3-targeted CAR. In certain embodiments, the CAR comprises (a) an extracellular antigen-binding domain comprising (i) a V_{H} that comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13, and (ii) a V_{L} that comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 40. In certain embodiments, the intracellular signaling domain comprises (i) a CD3ζ polypeptide comprising the amino acid sequence set forth in SEQ ID NO: 43, and (ii) a co-stimulatory signaling region comprising a CD28 polypeptide comprising amino acids 180 to 220 of SEQ ID NO: 40. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is designed as “MGA27128z”. In certain embodiments, the CAR comprises or consists of the amino acid sequence set forth in SEQ ID NO: 106. SEQ ID NO: 106 is provided below.

[SEQ ID NO: 106]
MALPVTALLLPLALLLHAARPEVQLVESGGGLVQPGGSLRLSCAASGFTFSSFGMHWVRQAPGKGLEWVAYIS
SDSSAIYYADTVKGRFTISRDNAKNSLYLQMNSLRDEDTAVYYCGRGRENIYYGSRLDYWGQGTTVTVSSGGG
GSGGGGSGGGGSDIQLTQSPSFLSASVGDRVTITCKASQNVDTNVAWYQQKPGKAPKALIYSASYRYSGVPSR
FSGSGSGTDFTLTISSLQPEDFATYYCQQYNNYPFTFGQGTKLEIKREQKLISEEDLAAAIEVMYPPPYLDNE
KSNGTIIHVKGKHLCPSPLFPGPSKPFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR

An exemplary nucleotide sequence encoding the amino acid sequence of SEQ ID NO: 106 is set forth in SEQ ID NO: 107, which is provided below.

[SEQ ID NO: 107]
ATGGCTCTCCCAGTGACTGCCCTACTGCTTCCCCTAGCGCTTCTCCTGCATGCAgccaggccgGAGGTCCAGC
TTGTTGAATCAGGGGGCGGCCTCGTCCAACCGGGGGGCTCACTGCGCTTGTCTTGCGCTGCCTCTGGATTTAC
CTTTAGTTCTTTCGGAATGCATTGGGTGCGGCAAGCACCCGGCAAGGGGTTGGAGTGGGTAGCGTATATAAGC
AGCGACTCCTCTGCCATCTACTACGCGGATACGGTGAAAGGCAGATTTACGATTAGTAGAGACAACGCTAAGA
ATTCATTGTACTTGCAAATGAACAGCCTCAGAGACGAGGATACCGCCGTGTACTATTGTGGAAGGGGCAGGGA
GAATATATACTATGGGTCAAGATTGGATTATTGGGGACAGGGGACGACAGTCACGGTGAGCAGTGGTGGAGGT
GGATCAGGTGGAGGTGGATCTGGTGGAGGTGGATCTGACATTCAGCTCACTCAGTCACCATCTTTTCTGTCAG
CGTCTGTGGGGGACAGAGTAACGATAACGTGCAAAGCGAGTCAGAACGTTGATACCAATGTGGCCTGGTATCA
GCAAAAGCCCGGTAAAGCCCCGAAGGCGCTGATCTATTCTGCCAGCTACCGATACTCAGGCGTTCCTTCACGC
TTTAGCGGGTCAGGCTCTGGTACCGACTTCACTCTTACGATCAGTTCATTGCAGCCCGAAGACTTCGCCACTT
ACTACTGCCAACAATATAATAACTACCCTTTCACGTTTGGCCAAGGCACCAAGTTGGAGATTAAGCGGGAACA
GAAACTGATCTCCGAGGAGGACCTCGCGGCCGCAATTGAAGTTATGTATCCTCCTCCTTACCTAGACAATGAG
AAGAGCAATGGAACCATTATCCATGTGAAAGGGAAACACCTTTGTCCAAGTCCCCTATTTCCCGGACCTTCTA
AGCCCTTTTGGGTGCTGGTGGTGGTTGGTGGAGTCCTGGCTTGCTATAGCTTGCTAGTAACAGTGGCCTTTAT
TATTTTCTGGGTGAGGAGTAAGAGGAGCAGGCTCCTGCACAGTGACTACATGAACATGACTCCCCGCCGCCCC
GGGCCCACCCGCAAGCATTACCAGCCCTATGCCCCACCACGCGACTTCGCAGCCTATCGCTCCAGAGTGAAGT
TCAGCAGGAGCGCAGACGCCCCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGGGGGGAAAGCCGAGAAGGAAG
AACCCTCAGGAAGGCCTGTACAATGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTCAGTACAGCCACCAAGGACACCTA
CGACGCCCTTCACATGCAGGCCCTGCCCCCTCGCTAA

In certain embodiments, the CAR is a B7-H3-targeted. In certain embodiments, the CAR comprises (a) an extracellular antigen-binding domain comprising (i) a V_{H} that comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13, and (ii) a V_{L} that comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; (b) a transmembrane domain comprising a CD8 polypeptide (e.g., a transmembrane domain of human C28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD8 polypeptide that comprises amino acids 137 to 209 of SEQ ID NO: 38. In certain embodiments, the intracellular signaling domain comprises (i) a CD3ζ polypeptide comprising the amino acid sequence set forth in SEQ ID NO: 42, and (ii) a co-stimulatory signaling region comprising a 4-1BB polypeptide comprising amino acids 214 to 255 of SEQ ID NO: 101. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is designed as “MGA271BBz”. In certain embodiments, the CAR comprises or consists of the amino acid sequence set forth in SEQ ID NO: 108. SEQ ID NO: 108 is provided below.

[SEQ ID NO: 108]
MALPVTALLLPLALLLHAARPEVQLVESGGGLVQPGGSLRLSCAASGFTFSSFGMHWVRQAPGKGLEWVAYIS
SDSSAIYYADTVKGRFTISRDNAKNSLYLQMNSLRDEDTAVYYCGRGRENIYYGSRLDYWGQGTTVTVSSGGG
GSGGGGSGGGGSDIQLTQSPSFLSASVGDRVTITCKASQNVDTNVAWYQQKPGKAPKALIYSASYRYSGVPSR
FSGSGSGTDFTLTISSLQPEDFATYYCQQYNNYPFTFGQGTKLEIKREQKLISEEDLAAAPTTTPAPRPPTPA
PTIASQPLSLRPEACRPAAGGAVHTRGLDFACDIYIWAPLAGTCGVLLLSLVITLYCNKRGRKKLLYIFKQPF
MRPVQTTQEEDGCSCRFPEEEEGGCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDALHMQALPPR

An exemplary nucleotide sequence encoding the amino acid sequence of SEQ ID NO: 108 is set forth in SEQ ID NO: 109, which is provided below.

[SEQ ID NO: 109]
ATGGCTCTCCCAGTGACTGCCCTACTGCTTCCCCTAGCGCTTCTCCTGCATGCAgccaggccgGAGGTCCAGC
TTGTTGAATCAGGGGGCGGCCTCGTCCAACCGGGGGGCTCACTGCGCTTGTCTTGCGCTGCCTCTGGATTTAC
CTTTAGTTCTTTCGGAATGCATTGGGTGCGGCAAGCACCCGGCAAGGGGTTGGAGTGGGTAGCGTATATAAGC
AGCGACTCCTCTGCCATCTACTACGCGGATACGGTGAAAGGCAGATTTACGATTAGTAGAGACAACGCTAAGA
ATTCATTGTACTTGCAAATGAACAGCCTCAGAGACGAGGATACCGCCGTGTACTATTGTGGAAGGGGCAGGGA
GAATATATACTATGGGTCAAGATTGGATTATTGGGGACAGGGGACGACAGTCACGGTGAGCAGTGGTGGAGGT
GGATCAGGTGGAGGTGGATCTGGTGGAGGTGGATCTGACATTCAGCTCACTCAGTCACCATCTTTTCTGTCAG
CGTCTGTGGGGGACAGAGTAACGATAACGTGCAAAGCGAGTCAGAACGTTGATACCAATGTGGCCTGGTATCA
GCAAAAGCCCGGTAAAGCCCCGAAGGCGCTGATCTATTCTGCCAGCTACCGATACTCAGGCGTTCCTTCACGC
TTTAGCGGGTCAGGCTCTGGTACCGACTTCACTCTTACGATCAGTTCATTGCAGCCCGAAGACTTCGCCACTT
ACTACTGCCAACAATATAATAACTACCCTTTCACGTTTGGCCAAGGCACCAAGTTGGAGATTAAGCGGGAACA
GAAACTGATCTCCGAGGAGGACCTCGCGGCCGCACCCACCACGACGCCAGCGCCGCGACCACCAACCCCGGCG
CCCACGATCGCGTCGCAGCCCCTGTCCCTGCGCCCAGAGGCGTGCCGGCCAGCGGCGGGGGGCGCAGTGCACA
CGAGGGGGCTGGACTTCGCCTGTGATATCTACATCTGGGCGCCCCTGGCCGGGACTTGTGGGGTCCTTCTCCT
GTCACTGGTTATCACCCTTTACTGCAACAAACGGGGCAGAAAGAAGCTCCTGTATATATTCAAACAACCATTT
ATGAGACCAGTACAAACTACTCAAGAGGAAGATGGCTGTAGCTGCCGATTTCCAGAAGAAGAAGAAGGAGGAT
GTGAACTGAGAGTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTGGCCGGGACCCTGAGATGGGG
GGAAAGCCGAGAAGGAAGAACCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGGCCTTTACCAGGGTCTCAGTAC
AGCCACCAAGGACACCTACGACGCCCTTCACATGCAGGCCCTGCCCCCTCGCTAA

### 3.3. TCR-Like Fusion Molecules

In certain embodiments, the antigen-recognizing receptor is a TCR-like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR-like fusion molecule comprises an antigen-binding chain that comprises an extracellular antigen-binding domain and a constant domain, wherein the TCR-like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide, and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex. In certain embodiments, the extracellular antigen-binding domain of the TCR-like fusion molecule is capable of dimerizing with another extracellular antigen-binding domain. In certain embodiments, the extracellular antigen-binding domain of the TCR-like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR-like fusion molecule comprises one or two immunoglobulin variable region(s). In certain embodiments, the extracellular antigen-binding domain of the TCR-like fusion molecule comprises a heavy chain variable region (V_{H}) of an antibody. In certain embodiments, the extracellular antigen-binding domain of the TCR-like fusion molecule comprises a light chain variable region (V_{L}) of an antibody. In certain embodiments, the extracellular antigen-binding domain of the TCR-like fusion molecule is capable of dimerizing with another extracellular antigen-binding domain. In certain embodiments, the extracellular antigen-binding domain of the TCR-like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR-like fusion molecule comprises a V_{L} of an antibody, wherein the V_{L} is capable of dimerizing with another extracellular antigen-binding domain comprising a V_{H} of the antibody and form a fragment variable (Fv).

### 4. Cells

The presently disclosed subject matter provides cells comprising a presently disclosed B7-H3-targeted antigen-recognizing receptor (e.g., one disclosed in Section 3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and TS T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed B7-H3-targeted antigen-recognizing receptor such that the cells express the antigen-recognizing receptor.

In certain embodiments, the presently disclosed cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPα, PD-L1, PD-L2, B37-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, the immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising a CAR and a soluble scFv that binds a polypeptide that has immunosuppressive activity or immunostimulatory activity are disclosed in International Patent Publication No. WO 2014/134165, which is incorporated by reference in its entirety.

In certain embodiments, the presently disclosed cell comprises a soluble single-chain variable fragment (scFv) that binds to PD-1. In certain embodiments, the soluble scFv comprises a V_{H} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof. SEQ ID NOs: 21-23 are provided in Table 3.

In certain embodiments, the soluble scFv comprises a V_{L} comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. SEQ ID NOs: 24-26 are provided in Table 3.

In certain embodiments, the soluble scFv comprises a V_{H} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof; and a V_{L} Comprising a CDR1 comprising the amino acid sequence set forth in SEQ ID NO: 24 or a conservative modification thereof, a CDR2 comprising the amino acid sequence set forth in SEQ ID NO: 25 or a conservative modification, and a CDR3 comprising the amino acid sequence set forth in SEQ ID NO: 26 or a conservative modification thereof.

In certain embodiments, the soluble scFv comprises a V_{H} comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23, a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the soluble scFv comprises a V_{H} Comprising an amino acid sequence that is at least about 80% (e.g., at least about 85%, at least about 90%, or at least about 95%) homologous or identical to the amino sequence set forth in SEQ ID NO: 27. For example, the soluble scFv comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27. In certain embodiments, the soluble scFv comprises a V_{H} comprising the amino sequence set forth in SEQ ID NO: 27. An exemplary nucleotide sequence encoding the amino acid sequence of SEQ ID NO: 27 is set forth in SEQ ID NO: 29. SEQ ID NOs: 27 and 29 are provided in Table 3 below.

In certain embodiments, the soluble scFv comprises a V_{L} Comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{L} Comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28. In certain embodiments, the extracellular antigen-binding domain comprises a V_{L} comprising the amino sequence set forth in SEQ ID NO: 28. An exemplary nucleotide sequence encoding the amino acid sequence of SEQ ID NO: 28 is set forth in SEQ ID NO: 30. SEQ ID NOs: 28 and 30 are provided in Table 3 below.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 28. In certain embodiments, the extracellular antigen-binding domain is an scFv. In certain embodiments, the scFv is designated as “E27”. In certain embodiments, the V_{H} and V_{L} are linked via a linker. In certain embodiments, the linker comprises the amino acid sequence set forth in SEQ ID NO: 31, SEQ ID NO: 32, SEQ ID NO: 33, SEQ ID NO: 34, SEQ ID NO: 35, or SEQ ID NO: 36.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 3

| CDRS | 1 | 2 | 3 |
|---|---|---|---|
| V_{H} | SYAMS | AISGSGGSTYYADSV | NYISMEDS |
| | [SEQ ID NO: 21] | KG | [SEQ ID NO: 23] |
| | | [SEQ ID NO: 22] | |
| V_{L} | GGNNIGSKSVH | YDSDRPS | QVWDSSSDYV |
| | [SEQ ID NO: 24] | [SEQ ID NO: 25] | [SEQ ID NO: 26] |

| | |
|---|---|
| Full V_{H} | EVQLVESGGGLIQPGGSLRLSCAASGFTFSSYAMSWVRQAPGKGLEWVS |
| | AISGSGGSTYYADSVKGRFTISRDNSKNTLYLQMNSLRAEDTAVYYCAR |
| | NYISMFDSWGQGTLVTVSS [SEQ ID NO: 27] |
| Full V_{L} | QSVLTQPPSVSVAPGKTARITCGGNNIGSKSVHWYQQRPGQAPVLVIYY |
| | DSDRPSGIPERFSGSNSGNTATLTISRVEAGDEADYYCQVWDSSSDYVF |
| | GIGTKVTVLG [SEQ ID NO: 28] |
| DNA | GAGGTGCAGCTGGTGGAGTCTGGAGGAGGCTTGATCCAGCCTGGGGGGT |
| for | CCCTGAGACTCTCCTGTGCAGCCTCTGGATTCACCTTTAGCAGCTATGC |
| Full V_{H} | CATGAGCTGGGTCCGCCAGGCTCCAGGGAAGGGGCTGGAGTGGGTCTCA |
| | GCTATTAGTGGTAGTGGTGGTAGCACATACTACGCAGACTCCGTGAAGG |
| | GCCGGTTCACCATCTCCAGAGACAATTCCAAGAACACGCTGTATCTGCA |
| | AATGAACAGCCTGAGAGCCGAGGACACGGCCGTATATTACTGTGCGCGC |
| | AACTACATCTCTATGTTCGATTCTTGGGGTCAAGGTACTCTGGTGACCG |
| | TCTCCTCA [SEQ ID NO: 29] |
| DNA | CAGTCTGTGCTGACTCAGCCACCCTCAGTGTCAGTGGCCCCAGGAAAGA |
| for | CGGCCAGGATTACCTGTGGGGGAAACAACATTGGAAGTAAAAGTGTGCA |
| Full V_{L} | CTGGTACCAGCAGAGGCCAGGCCAGGCCCCTGTGCTGGTCATCTATTAT |
| | GATAGCGACCGGCCCTCAGGGATCCCTGAGCGATTCTCTGGCTCCAACT |
| | CTGGGAACACGGCCACCCTGACCATCAGCAGGGTCGAAGCCGGGGATGA |
| | GGCCGACTATTACTGTCAGGTGTGGGATAGTAGTAGTGATTATGTCTTC |
| | GGAATTGGGACCAAGGTCACCGTCCTAGGT [SEQ ID NO: 30] |

### 4.1. Exemplified Cells

In certain embodiments, the cell comprises an antigen-recognizing receptor. In certain embodiments, the antigen-recognizing receptor is a CAR. In certain embodiments, the CAR is a B7-H-3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3 polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 40. In certain embodiments, the intracellular signaling domain comprises (i) a CD3 polypeptide comprising the amino acid sequence set forth in SEQ ID NO: 43, and (ii) a co-stimulatory signaling region comprising a CD28 polypeptide comprising amino acids 180 to 220 of SEQ ID NO: 40. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is “M3028z”.

In certain embodiments, the CAR comprises or consists of the amino acid sequence set forth in SEQ ID NO: 102. In certain embodiments, the cell is designated as “GMB28z”.

In certain embodiments, the cell comprises i) an antigen-recognizing receptor, and ii) a soluble scFv. In certain embodiments, the antigen-recognizing receptor is a CAR. In certain embodiments, the cell comprises an antigen-recognizing receptor. In certain embodiments, the antigen-recognizing receptor is a CAR. In certain embodiments, the CAR is a B7-H3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 40. In certain embodiments, the intracellular signaling domain comprises (i) a CD3ζ polypeptide comprising the amino acid sequence set forth in SEQ ID NO: 43, and (ii) a co-stimulatory signaling region comprising a CD28 polypeptide comprising amino acids 180 to 220 of SEQ ID NO: 40. In certain embodiments, the V_{H} and V_{L} are linked via a linker comprising or consisting of the amino acid sequence set forth in SEQ ID NO: 32. In certain embodiments, the V_{H} and V_{L} are positioned from the N- to the C-terminus: V_{L}-V_{H}. In certain embodiments, the CAR is “M3028z”.

In certain embodiments, the CAR comprises or consists of the amino acid sequence set forth in SEQ ID NO: 102. In certain embodiments, the soluble scFv binds to PD-1. In certain embodiments, the soluble scFv comprises a V_{H} that comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23, and a V_{L} that comprises a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26. In certain embodiments, the soluble scFv comprises a V_{H} that comprises the amino acid sequence set forth in SEQ ID NO: 27 and a V_{L} that comprises the amino acid sequence set forth in SEQ ID NO: 29. In certain embodiments, the cell is designated as “GMB28z-E27”.

### 5. Nucleic Acid Compositions and Vectors

The present discloses subject matter provides a nucleic acid encoding a presently disclosed B7-H3-targeted antigen-recognizing receptor (e.g., one disclosed in Section 3). Further provided are nucleic acid compositions comprising the nucleic acids disclosed herein. Also provided are cells comprising such nucleic acid compositions.

In certain embodiments, the nucleic acid composition further comprises a promoter that is operably linked to the presently disclosed B7-H3-targeted antigen-recognizing receptor.

In certain embodiments, the promoter is endogenous or exogenous. In certain embodiments, the exogenous promoter is selected from an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. In certain embodiments, the inducible promoter is selected from a NFAT transcriptional response element (TRE) promoter, a CD69 promoter, a CD25 promoter, and an IL-2 promoter.

In certain embodiments, the nucleic acid encodes a presently disclosed B7-H3-targeted antigen-recognizing receptor and a soluble single-chain variable fragment (scFv) that binds a polypeptide that has immunosuppressive activity or immunostimulatory activity. In certain embodiments, the antigen-recognizing receptor and the soluble scFv are separated by a self-cleavage peptide, e.g., a 2A-peptide. In certain embodiments, the antigen-recognizing receptor and the soluble scFv are separated by a P2A peptide. In certain embodiments, the peptide comprises the amino acid sequence set forth in SEQ ID NO: 110.

[SEQ ID NO: 110]
GSGATNFSLLKQAGDVEENPGP

An exemplary nucleotide sequence encoding the amino acid sequence of SEQ ID NO: 110 is set forth in SEQ ID NO: 111, which is provided below.

[SEQ ID NO: 111]
GGATCTGGAGCAACAAACTTCTCACTACTCAAACAAGCAGGTGACGTGG
AGGAGAATCCCGGACCC

The compositions and nucleic acid compositions can be administered to subjects or and/delivered into cells by art-known methods or as described herein. Genetic modification of a cell (e.g., a T cell or an NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed B7-H3-targeted antigen-recognizing receptor (e.g., a CAR), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-xB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### 5.1. Methods of Delivering

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of 20 the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding an hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″′-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5.2. Exemplified Nucleic Acids

In certain embodiments, the nucleic acid encodes a B7-H3-targeted CAR. In certain embodiments, the CAR is M3028z. In certain embodiments, the nucleic acid comprises or consists of a nucleotide sequence that is at least about 85%, about 90%, about 95%, about 96%, about 97%, about 98%, about 99%, or about 100% identical to the nucleotide sequence set forth in SEQ ID NO: 103. In certain embodiments, the nucleic acid comprises or consists of the nucleotide sequence set forth in SEQ ID NO: 103.

In certain embodiments, the nucleic acid encodes i) a B7-H3-targeted CAR, and ii) a soluble scFv that binds to PD-1. In certain embodiments, the CAR is M3028z. In certain embodiments, the soluble scFv is E27. In certain embodiments, the nucleic acid encodes an amino acid sequence that is at least about 85%, about 90%, about 95%, about 96%, about 97%, about 98%, about 99%, or about 100% identical to the amino acid sequence set forth in SEQ ID NO: 112. In certain embodiments, the amino acid comprises or consists of the amino acid sequence set forth in SEQ ID NO: 112. SEQ ID NO: 112 is provided below:

[SEQ ID NO: 112]
MALPVTALLLPLALLLHAARPEVQLQQSGPELVKPGASVKMSCKASGYTFTNYVMHWVKQKPGQGLEWIGYIN
PYNDDVKYNEKFKGKATQTSDKSSSTAYMELSSLTSEDSAVYYCARWGYYGSPLYYFDYWGQGTTLTVSSGGG
GSGGGGSGGGGSQIVLSQSPTILSASPGEKVTMTCRASSRLIYMHWYQQKPGSSPKPWIYATSNLASGVPARF
SGSGSGTSYSLTISRVEAEDAATYYCQQWNSNPPTFGTGTKLELKREQKLISEEDLAAAIEVMYPPPYLDNEK
SNGTIIHVKGKHLCPSPLFPGPSKPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPRGSGATNFSLLKQAG
DVEENPGPMETDTLLLWVLLLWVPGSTGQSVLTQPPSVSVAPGKTARITCGGNNIGSKSVHWYQQRPGQAPVL
VIYYDSDRPSGIPERFSGSNSGNTATLTISRVEAGDEADYYCQVWDSSSDYVFGIGTKVTVLGSRGGGGSGGG
GSGGGGSLEMAEVQLVESGGGLIQPGGSLRLSCAASGFTFSSYAMSWVRQAPGKGLEWVSAISGSGGSTYYAD
SVKGRFTISRDNSKNTLYLQMNSLRAEDTAVYYCARNYISMFDSWGQGTLVTVSSTSGQAGQHHHHHHGAYPY
DVPDYAS

In certain embodiments, the nucleic acid encodes i) a B7-H3-targeted CAR, and ii) a soluble scFv that binds to PD-1. In certain embodiments, the CAR is M3028z. In certain embodiments, the soluble scFv is E27. In certain embodiments, the nucleic acid comprises or consists of a nucleotide sequence that is at least about 85%, about 90%, about 95%, about 96%, about 97%, about 98%, about 99% or about 100% identical to the amino acid sequence set forth in SEQ ID NO: 113. In certain embodiments, the nucleic acid comprises or consists of the nucleotide sequence set forth in SEQ ID NO: 113. SEQ ID NO: 113 is provided below:

[SEQ ID NO: 113]
ATGGCTCTCCCAGTGACTGCCCTACTGCTTCCCCTAGCGCTTCTCCTGCATGCAgccaggccgGAAGTACAGC
TTCAGCAGTCAGGTCCAGAACTGGTCAAGCCAGGAGCATCCGTAAAGATGTCCTGTAAGGCCAGCGGTTACAC
ATTTACGAATTACGTCATGCATTGGGTAAAGCAGAAGCCAGGCCAAGGTCTTGAATGGATCGGATACATTAAC
CCATACAATGACGATGTGAAATACAATGAGAAGTTTAAGGGCAAAGCTACTCAAACGTCAGATAAATCTTCCA
GCACAGCTTACATGGAGCTGTCTTCCCTGACAAGCGAGGATAGCGCAGTTTATTACTGCGCCCGATGGGGTTA
TTATGGGTCACCTCTTTATTATTTTGACTATTGGGGTCAAGGGACGACCTTGACCGTATCATCAGGTGGAGGT
GGATCAGGTGGAGGTGGATCTGGTGGAGGTGGATCTCAGATCGTGTTGAGTCAGAGTCCTACCATATTGTCAG
CGAGTCCGGGAGAGAAGGTAACTATGACCTGTCGGGCTTCTAGCCGGCTGATATATATGCACTGGTACCAACA
AAAGCCGGGCTCATCACCTAAACCTTGGATATATGCTACAAGTAATCTTGCGTCAGGCGTCCCAGCCCGGTTC
TCCGGCTCCGGTTCAGGGACAAGTTACTCACTGACTATAAGTCGCGTTGAAGCCGAGGATGCCGCCACGTATT
ATTGTCAGCAATGGAACAGTAACCCTCCCACATTTGGCACGGGGACTAAGCTGGAATTGAAAAGGGAACAGAA
ACTGATCTCCGAGGAGGACCTCGCggccgcaattgaagttatgtatcctcctccttacctagacaatgagaag
agcaatggaaccattatccatgtgaaagggaaacacctttgtccaagtcccctatttcccggaccttctaagc
ccttttgggtgctggtggtggttggtggagtcctggcttgctatagcttgctagtaacagtggcctttattat
tttctgggtgaggagtaagaggagcaggctcctgcacagtgactacatgaacatgactccccgccgccccggg
cccacccgcaagcattaccagccctatgccccaccacgcgacttcgcagcctatcgctccagagtgaagttca
gcaggagcgcagaCGcccccgcgtaccagcagggccagaaccagctctataacgagctcaatctaggacgaag
agaggagtacgatgttttggacaagagacgtggccgggaccctgagatggggggaaagccgagaaggaagaac
cctcaggaaggcctgtacaatgaactgcagaaagataagatggcggaggcctacagtgagattgggatgaaag
gcgagcgccggaggggcaaggggcacgatggcctttaccagggtctcagtacagccaccaaggacacctacga
cgcccttcacatgcaggccctgccccctcgcggatctggagcaacaaacttctcactactcaaacaagcaggt
gacgtggaggagaatcccggacccatgGAAACCGACACCCTGCTGCTGTGGGTGCTGCTGCTGTGGGTGCCAG
GATCTACAGGACAGTCTGTGCTGACTCAGCCACCCTCAGTGTCAGTGGCCCCAGGAAAGACGGCCAGGATTAC
CTGTGGGGGAAACAACATTGGAAGTAAAAGTGTGCACTGGTACCAGCAGAGGCCAGGCCAGGCCCCTGTGCTG
GTCATCTATTATGATAGCGACCGGCCCTCAGGGATCCCTGAGCGATTCTCTGGCTCCAACTCTGGGAACACGG
CCACCCTGACCATCAGCAGGGTCGAAGCCGGGGATGAGGCCGACTATTACTGTCAGGTGTGGGATAGTAGTAG
TGATTATGTCTTCGGAATTGGGACCAAGGTCACCGTCCTAGGTTCTAGAGGTGGTGGTGGTAGCGGCGGCGGC
GGCTCTGGTGGTGGTGGATCCCTCGAGATGGCCGAGGTGCAGCTGGTGGAGTCTGGAGGAGGCTTGATCCAGC
CTGGGGGGTCCCTGAGACTCTCCTGTGCAGCCTCTGGATTCACCTTTAGCAGCTATGCCATGAGCTGGGTCCG
CCAGGCTCCAGGGAAGGGGCTGGAGTGGGTCTCAGCTATTAGTGGTAGTGGTGGTAGCACATACTACGCAGAC
TCCGTGAAGGGCCGGTTCACCATCTCCAGAGACAATTCCAAGAACACGCTGTATCTGCAAATGAACAGCCTGA
GAGCCGAGGACACGGCCGTATATTACTGTGCGCGCAACTACATCTCTATGTTCGATTCTTGGGGTCAAGGTAC
TCTGGTGACCGTCTCCTCAACTAGTGGCCAGGCCGGCCAGCACCATCACCATCACCATGGCGCATACCCGTAC
GACGTTCCGGACTACGCTTCTTAG

### 6. Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, B7-H3, CD8, CD28, 4-1BB, and CD3ζ,). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 7. Formulations and Administration

The presently disclosed subject matter provides compositions comprising the presently disclosed cells. In certain embodiments, the compositions are pharmaceutical compositions further comprising a pharmaceutically acceptable carrier. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like.

The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for treating or ameliorating a disease or disorder. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. In certain embodiments, about 2.5×10⁵ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intravitreal administration, intrapleural administration, intraosseous administration, intraperitoneal administration, pleural administration, and direct administration to the subject.

Additionally or alternatively, the presently disclosed subject matter also provides compositions comprising lipid nanoparticles (e.g., described in Section 5.1) including a nucleic acid or a nucleic acid composition disclosed herein. Compositions comprising the presently disclosed lipid nanoparticles can be conveniently provided as sterile and/or pyrogen-free. Compositions can be prepared to meet the standards of the United States Pharmacopoeia (USP), the European Pharmacopoeia (EP), the British Pharmacopoeia, and/or the International Pharmacopoeia.

Compositions including the presently disclosed lipid nanoparticles can include pharmaceutically acceptable excipients. Non-limiting examples of pharmaceutically acceptable excipients include inert diluents, dispersing agents, granulating agents, surface active agents, emulsifiers, disintegrating agents, binding agents, preservatives, buffering agents, lubricating agents, and/or oils. Furthermore, excipients such as cocoa butter and suppository waxes, coloring agents, coating agents, sweetening, flavoring, and/or perfuming agents can be present in the composition.

In certain embodiments, compositions including the presently disclosed lipid nanoparticles can be prepared as injectable preparations. These injectable preparations can include pharmaceutically acceptable vehicles and solvents including, without any limitation, water, Ringer's solution, U.S.P., isotonic sodium chloride solution, and/or oils (e.g., oleic acid). In certain embodiments, injectable preparations comprising the presently disclosed lipid nanoparticles can include a liquid suspension of crystalline or amorphous material with poor water solubility. Use of these poor water solubility materials allows to slow absorption from subcutaneous or intramuscular injection. Alternatively or additionally, compositions including the presently disclosed lipid nanoparticles can be prepared for rectal or vaginal administration, oral administration, topical and/or transdermal administration, intradermal administration, pulmonary administration, nasal administration, buccal administration, or ophthalmic administration. Additional information on various ways for formulating and preparing pharmaceutical compositions including the presently disclosed lipid nanoparticles can be found in Remington: The Science and Practice of Pharmacy, 22nd Edition, A. R. Gennaro, Lippincott, Williams & Wilkins, Baltimore, Md., 2012.

In certain embodiments, the compositions including the presently disclosed lipid nanoparticles can be formulated for controlled release or sustained release. As used herein, the term “controlled release” refers to a pharmaceutical composition or compound release profile that conforms to a particular pattern of release to effect a therapeutic outcome. As used herein, the term “sustained release” refers to a pharmaceutical composition or compound that conforms to a release rate over a specific period of time. The period of time may include, but is not limited to, hours, days, weeks, months and years.

Compositions comprising the presently disclosed lipid nanoparticles can be provided systemically or directly to a subject for inducing and/or enhancing an immune response to an antigen and/or treating and/or preventing a tumor, e.g., a tumor associated with MUC16. In certain embodiments, the presently disclosed lipid nanoparticles or compositions comprising thereof are provided in vivo to immunoresponsive cells. In certain embodiments, the presently disclosed lipid nanoparticles or compositions comprising thereof are directly injected into an organ of interest (e.g., an organ affected by a neoplasia). Alternatively, the presently disclosed lipid nanoparticles or compositions comprising thereof are provided indirectly to the organ of interest, for example, by administration into the circulatory system (e.g., the tumor vasculature). In certain embodiments, the presently disclosed lipid nanoparticles or compositions comprising thereof are provided ex vivo to immunoresponsive cells. Expansion and differentiation agents can be provided prior to, during or after administration of the lipid nanoparticles or compositions to increase production of cells (e.g., T cells or NK cells) ex vivo or in vivo.

The presently disclosed lipid nanoparticles can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of the presently disclosed lipid nanoparticles are administered to a subject. In certain embodiments, between about 0.005 mg/kg to about 2.5 mg/kg, from about 0.1 mg/kg to about 1 mg/kg, or from about 0.05 mg/kg to about 1 mg/kg of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art. Dosages can be readily adjusted by those skilled in the art (e.g., a decrease in purity may require an increase in dosage).

### 8. Methods of Treatment

The presently disclosed cells and compositions comprising thereof can be used for treating or ameliorating a disease or disorder in a subject. In certain embodiments, the disease or disorder is associated with B7-H3. In certain embodiments, the disease or disorder is associated with overexpression of B7-H3.

In certain embodiments, the method comprises administering to a subject in need thereof the presently disclosed cells or compositions comprising thereof. In certain embodiments, the cell is a T cell. The T cell can be a CD4⁺ T cell or a CD8⁺ T cell. In certain embodiments, the T cell is a CD4⁺ T cell.

Additionally or alternatively, the presently disclosed lipid nanoparticles and compositions comprising thereof can be used for treating or ameliorating a disease or disorder in a subject. In certain embodiments, the disease or disorder is associated with B7-H3. In certain embodiments, the disease or disorder is associated with overexpression of B7-H3. In certain embodiments, the method comprises administering to a subject in need thereof the presently disclosed lipid nanoparticles or compositions comprising thereof.

For treatment, the amount administered is an amount effective in producing the desired effect. An effective amount can be provided in one or a series of administrations. An effective amount can be provided in a bolus or by continuous perfusion.

In certain embodiments, the disease or disorder is a tumor. In certain embodiments, the presently disclosed cells and compositions can reduce tumor burden, reduce the number of tumor cells, reduce tumor size, and/or eradicate the tumor in the subject, and/or increase or lengthen survival of the subject. In certain embodiments, the tumor is cancer. In certain embodiments, the tumor is a metastatic cancer. Non-limiting examples of diseases and disorders include osteosarcoma, rhabdomyosarcoma, Ewing's sarcoma, neuroblastoma, desmoplastic small round cell tumor, synovial sarcoma, undifferentiated sarcoma, adrenocortical carcinoma, hepatoblastoma, Wilms tumor, rhabdoid tumor, high-grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma, head and neck cancers, leiomyosarcoma, and melanoma. In certain embodiments, the tumor is high-grade glioma (glioblastoma multiforme). In certain embodiments, the tumor is melanoma.

Further modification can be introduced to the B7-H3-specific CAR-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the B7-H3-specific CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the B7-H3-specific CAR. The suicide gene can be included within the vector comprising nucleic acids encoding the presently disclosed B7-H3-specific CARs. The incorporation of a suicide gene into a presently disclosed B7-H3-specific CAR gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

### 9. Kits

The presently disclosed subject matter provides kits for or ameliorating a disease or disorder in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding a presently disclosed B7-H3-targeted antigen-recognizing receptor (e.g., a CAR).

If desired, the cells and/or nucleic acid molecules are provided together with instructions for administering the cells or nucleic acid molecules to a subject having or at risk of developing a disease or disorder. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 10. Exemplary Embodiments

Embodiment 1. An immunoresponsive cell comprising an antigen-recognizing receptor comprising an extracellular antigen-binding domain, a transmembrane domain, and an intracellular signaling domain, wherein the extracellular antigen-binding domain specifically binds to B7-H3 and comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; or
(b) a heavy chain variable region comprising a CDR1 comprising the amino acid sequence set forth in SEQ ID NO: 11 or a conservative modification thereof, a CDR2 comprising an amino acid sequence set forth in SEQ ID NO: 12 or a conservative modification thereof, and a CDR3 comprising the amino acid sequence set forth in SEQ ID NO: 13 or a conservative modification thereof; and a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof.

Embodiment 2. The immunoresponsive cell of embodiment 1, wherein the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

Embodiment 3. The immunoresponsive cell of embodiment 1 or 2, wherein the extracellular antigen-binding domain comprises a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

Embodiment 4. The immunoresponsive cell of any one of embodiments 1-3, wherein the extracellular antigen-binding domain is a single-chain variable fragment (scFv).

Embodiment 5. The immunoresponsive cell of any one of embodiments 1-4, wherein the extracellular antigen-binding domain is a human scFv.

Embodiment 6. The immunoresponsive cell of any one of embodiments 1-3, wherein the extracellular antigen-binding domain is a Fab, which is optionally crosslinked.

Embodiment 7. The immunoresponsive cell of any one of embodiments 1-3, wherein the extracellular antigen-binding domain is a F(ab)2.

Embodiment 8. The immunoresponsive cell of any one of embodiments 1-7, wherein the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7 or SEQ ID NO: 17.

Embodiment 9. The immunoresponsive cell of any one of embodiments 1-8, wherein the extracellular antigen-binding domain comprises a heavy chain variable region comprising the amino acid sequence set forth in SEQ ID NO: 7 or SEQ ID NO: 17.

Embodiment 10. The immunoresponsive cell of any one of embodiments 1-9, wherein the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or SEQ ID NO: 18.

Embodiment 11. The immunoresponsive cell of any one of embodiments 1-10, wherein the extracellular antigen-binding domain comprises a light chain variable region comprising the amino acid sequence set forth in SEQ ID NO: 8 or SEQ ID NO: 18.

Embodiment 12. The immunoresponsive cell of any one of embodiments 1-11, wherein the extracellular antigen-binding domain comprises:
(a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7 or SEQ ID NO: 17; and
(b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or SEQ ID NO: 18.

Embodiment 13. The immunoresponsive cell of any one of embodiments 1-12, wherein the extracellular antigen-binding domain comprises: (a) a heavy chain variable region comprising the amino acid sequence set forth in SEQ ID NO: 7 or SEQ ID NO: 17; and (b) a light chain variable region comprising the amino acid sequence set forth in SEQ ID NO: 8 or SEQ ID NO: 18.

Embodiment 14. The immunoresponsive cell of any one of embodiments 1-13, wherein the extracellular antigen-binding domain comprises: (a) a heavy chain variable region comprising the amino acid sequence set forth in SEQ ID NO: 7, and a light chain variable region comprising the amino acid sequence set forth in SEQ ID NO: 8; or (b) a heavy chain variable region comprising the amino acid sequence set forth in SEQ ID NO: 17, and a light chain variable region comprising the amino acid sequence set forth in SEQ ID NO: 18.

Embodiment 15. The immunoresponsive cell of any one of embodiments 1-14, wherein the extracellular antigen-binding domain comprises a linker between a heavy chain variable region and a light chain variable region of the extracellular antigen-binding domain.

Embodiment 16. The immunoresponsive cell of embodiment 15, wherein the linker comprises or consists of the amino acid sequence set forth in SEQ ID NO: 31, SEQ ID NO: 32, SEQ ID NO: 33, SEQ ID NO: 34, SEQ ID NO: 35, or SEQ ID NO: 36.

Embodiment 17. The immunoresponsive cell of any one of embodiments 1-16, wherein the extracellular antigen-binding domain comprises a signal peptide that is covalently joined to the 5′ terminus of the extracellular antigen-binding domain.

Embodiment 18. The immunoresponsive cell of any one of embodiments 1-17, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

Embodiment 19. The immunoresponsive cell of any one of embodiments 1-18, wherein the intracellular signaling domain comprises a CD3ζ polypeptide.

Embodiment 20. The immunoresponsive cell of embodiment 19, wherein the CD3ζ polypeptide comprises or consists of the amino acid sequence set forth in SEQ ID NO: 43.

Embodiment 21. The immunoresponsive cell of any one of embodiments 1-20, wherein the intracellular signaling domain further comprises at least one co-stimulatory signaling region.

Embodiment 22. The immunoresponsive cell of embodiment 21, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

Embodiment 23. The immunoresponsive cell of embodiment 22, wherein the at least one co-stimulatory signaling region comprises a CD28 polypeptide.

Embodiment 24. The immunoresponsive cell of embodiment 23, wherein the CD28 polypeptide comprises or consists of amino acids 180 to 220 of SEQ ID NO: 40.

Embodiment 25. The immunoresponsive cell of embodiment 23, wherein the CD28 polypeptide comprises a mutated YMNM motif.

Embodiment 26. The immunoresponsive cell of any one of embodiments 23-25, wherein the CD28 polypeptide comprises or consists of the amino acid sequence set forth in SEQ ID NO: 96, SEQ ID NO: 97, SEQ ID NO: 98, SEQ ID NO: 99, or SEQ ID NO: 100.

Embodiment 27. The immunoresponsive cell of embodiment 22, wherein the at least one co-stimulatory signaling region comprises a 4-1BB polypeptide.

Embodiment 28. The immunoresponsive cell of embodiment 27, wherein the 4-1BB polypeptide comprises or consists of amino acids 214 to 255 of SEQ ID NO: 101.

Embodiment 29. The immunoresponsive cell of any one of embodiments 1-28, wherein the antigen-recognizing receptor is a chimeric antigen receptor (CAR), or a T-cell like fusion protein.

Embodiment 30. The immunoresponsive cell of any one of embodiments 1-29, wherein the antigen-recognizing receptor is a CAR.

Embodiment 31. The immunoresponsive cell of embodiment 30, wherein the CAR comprises the amino acid sequence set forth in SEQ ID NO: 102, SEQ ID NO: 104, or SEQ ID NO: 106.

Embodiment 32. The immunoresponsive cell of embodiment 30 or 31, wherein the CAR comprises the amino acid sequence set forth in SEQ ID NO: 102.

Embodiment 33. The immunoresponsive cell of any one of embodiments 1-32, wherein the antigen-recognizing receptor is recombinantly expressed.

Embodiment 34. The immunoresponsive cell of any one of embodiments 1-33, wherein the antigen-recognizing receptor is expressed from a vector.

Embodiment 35. The immunoresponsive cell of embodiment 34, wherein the vector is a γ-retroviral vector.

Embodiment 36. The immunoresponsive cell of any one of embodiments 1-35, wherein the antigen-recognizing receptor is constitutively expressed on the surface of the cell.

Embodiment 37. The immunoresponsive cell of any one of embodiments 1-36, further comprising a soluble scFv.

Embodiment 38. The immunoresponsive cell of embodiment 37, wherein the soluble scFv comprises a heavy chain variable region comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof; and a light chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof.

Embodiment 39. The immunoresponsive cell of embodiment 37 or 38, wherein the soluble scFv comprises (a) a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and (b) a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

Embodiment 40. The immunoresponsive cell of any one of embodiments 37-39, wherein the soluble scFv comprises a heavy chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27.

Embodiment 41. The immunoresponsive cell of any one of embodiments 37-40, wherein the soluble scFv comprises a light chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28.

Embodiment 42. The immunoresponsive cell of any one of embodiments 37-41, wherein the soluble scFv comprises: (a) a heavy chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27; and
(b) a light chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28.

Embodiment 43. The immunoresponsive cell of any one of embodiments 1-42, wherein the cell comprises a polypeptide comprising an amino acid sequence that is at least about 85%, about 90%, about 95%, about 96%, about 97%, about 98%, or about 99% to the amino acid sequence set forth in set forth in SEQ ID NO: 112.

Embodiment 44. The immunoresponsive cell of any one of embodiments 1-43, wherein the cell comprises a polypeptide comprising the amino acid sequence set forth in set forth in SEQ ID NO: 112.

Embodiment 45. The immunoresponsive cell of any one of embodiments 1-44, wherein the cell is a cell of the lymphoid lineage or a cell of the myeloid lineage.

Embodiment 46. The immunoresponsive cell of any one of embodiments 1-45, wherein the cell is selected from the group consisting of a T cell, a Natural Killer (NK) cell, and a stem cell from which a lymphoid cell may be differentiated.

Embodiment 47. The immunoresponsive cell of any one of embodiments 1-46, wherein the cell is a T cell.

Embodiment 48. The immunoresponsive cell of embodiment 46 or 47, wherein the T cell is a cytotoxic T lymphocyte (CTL) or a regulatory T cell.

Embodiment 49. The immunoresponsive cell of any one of embodiments 1-46, wherein the cell is a stem cell.

Embodiment 50. The immunoresponsive cell of embodiment 49, wherein the stem cell is a pluripotent stem cell.

Embodiment 51. The immunoresponsive cell of embodiment 50, wherein the pluripotent stem cell is an embryoid stem cell or an induced pluripotent stem cell.

Embodiment 52. The immunoresponsive cell of any one of embodiments 1-46, wherein the cell is a NK cell.

Embodiment 53. A composition comprising the cell of any one of embodiments 1-52.

Embodiment 54. The composition of embodiment 53, which is a pharmaceutical composition further comprising a pharmaceutically acceptable carrier.

Embodiment 55. A nucleic acid encoding:
(a) an antigen-recognizing receptor comprising an extracellular antigen-binding domain, a transmembrane domain, and an intracellular signaling domain, wherein the extracellular antigen-binding domain specifically binds to B7-H3 and comprises:
(i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
(ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and
(b) a soluble scFv comprising a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

Embodiment 56. The nucleic acid of embodiment 55, wherein the nucleic acid encodes an amino acid sequence that is at least about 85%, about 90%, about 95%, about 96%, about 97%, about 98%, or about 99% identical to the amino acid sequence set forth in SEQ ID NO: 112.

Embodiment 57. A vector comprising the nucleic acid of embodiment 55 or 56.

Embodiment 58. The vector of embodiment 57, wherein the vector is a 7-retroviral vector.

Embodiment 59. The vector of embodiment 57 or 58, wherein the vector is a lentiviral vector.

Embodiment 60. A lipid nanoparticle comprising the nucleic acid of embodiment 55 or 56, or the vector of any one of claims 57-59.

Embodiment 61. A cell comprising the nucleic acid of embodiment 55 or 56, the vector of any one of claims 57-59, or the lipid nanoparticle of claim 60.

Embodiment 62. A method of producing an immunoresponsive cell targeting B7-H3, the method comprising introducing into the cell the nucleic acid of embodiment 55 or 56, the vector of any one of claims 57-59, or the lipid nanoparticle of claim 60.

Embodiment 63. A method of treating or ameliorating a disease or disorder in a subject, comprising administering to the subject an effective amount of the presently disclosed cell of any one of embodiments 1-52 or the composition of any one of claims 53-54.

Embodiment 64. The method of embodiment 63, wherein the disease or disorder is a tumor.

Embodiment 65. The method of embodiment 63 or 64, wherein the tumor is cancer.

Embodiment 66. The method of any one of embodiments 63-65, wherein the disease or disorder is selected from the group consisting of osteosarcoma, rhabdomyosarcoma, Ewing's sarcoma, neuroblastoma, desmoplastic small round cell tumor, synovial sarcoma, undifferentiated sarcoma, adrenocortical carcinoma, hepatoblastoma, Wilms tumor, rhabdoid tumor, high-grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma, head and neck cancers, leiomyosarcoma, and melanoma.

Embodiment 67. The method of any one of embodiments 63-66, wherein the tumor is high-grade glioma (glioblastoma multiforme).

Embodiment 68. The method of any one of embodiments 63-66, wherein the tumor is melanoma.

Embodiment 69. The method of any one of embodiments 63-68, wherein the subject is a human.

Embodiment 70. A kit for treating or ameliorating a disease or disorder in a subject, comprising the cell of any one of embodiments 1-52 or the composition of any one of claims 53-54.

Embodiment 71. The kit of embodiment 70, wherein the kit further comprises written instructions for using the cell or composition for treating or ameliorating a disease or disorder in a subject.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1

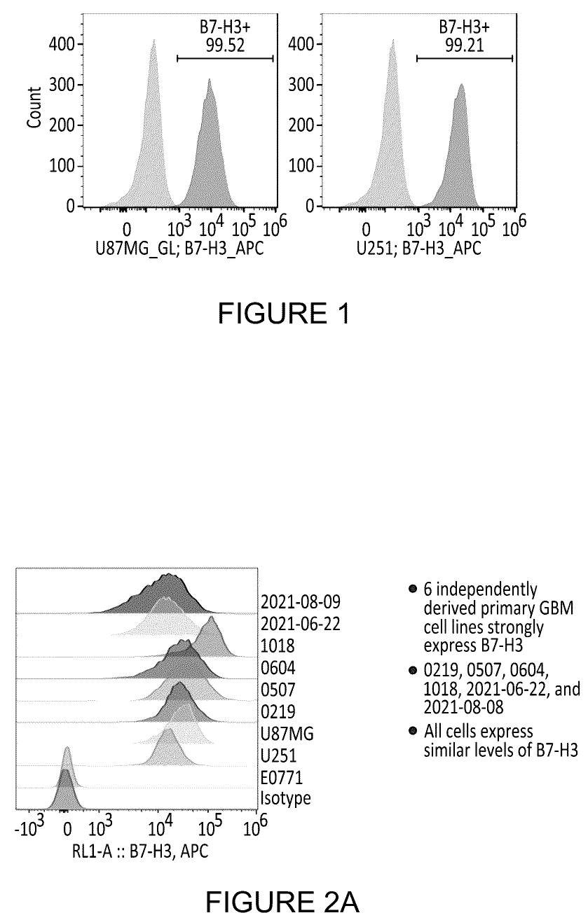

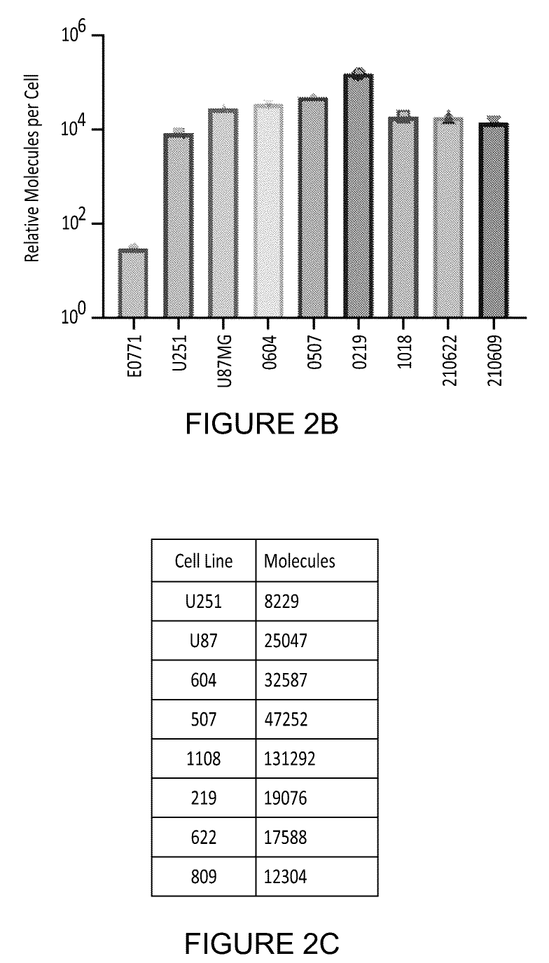

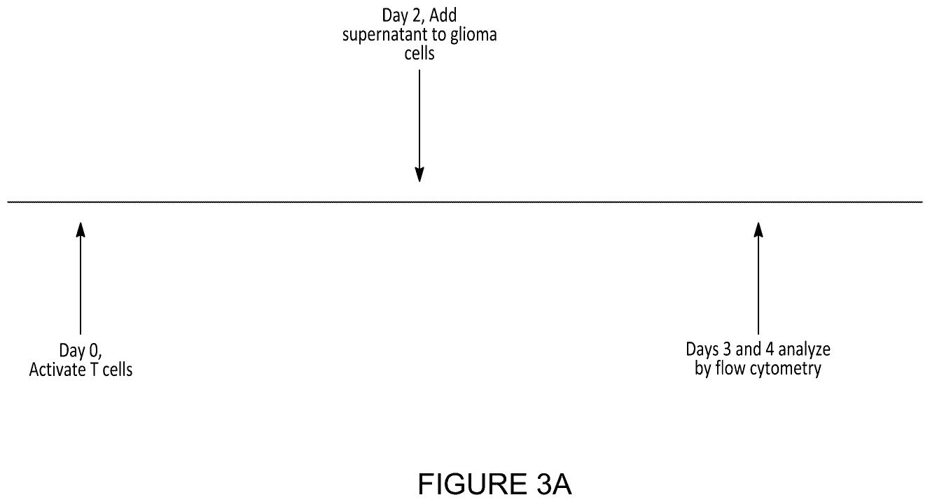

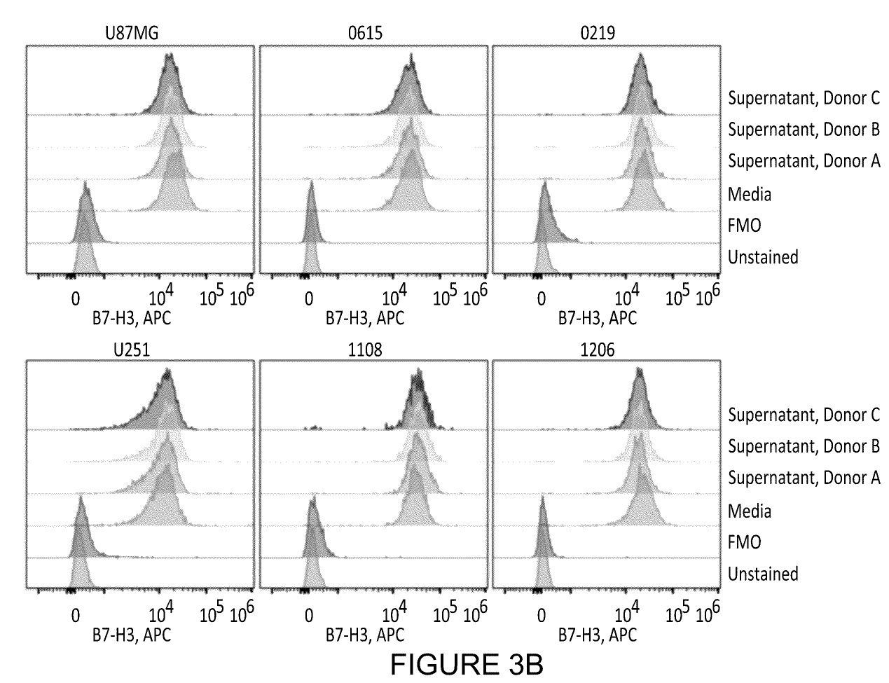

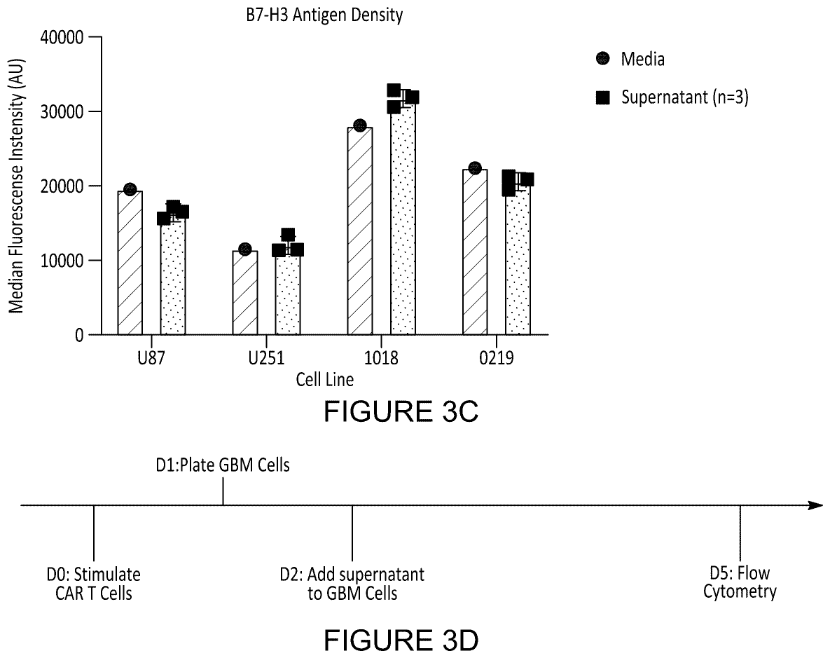

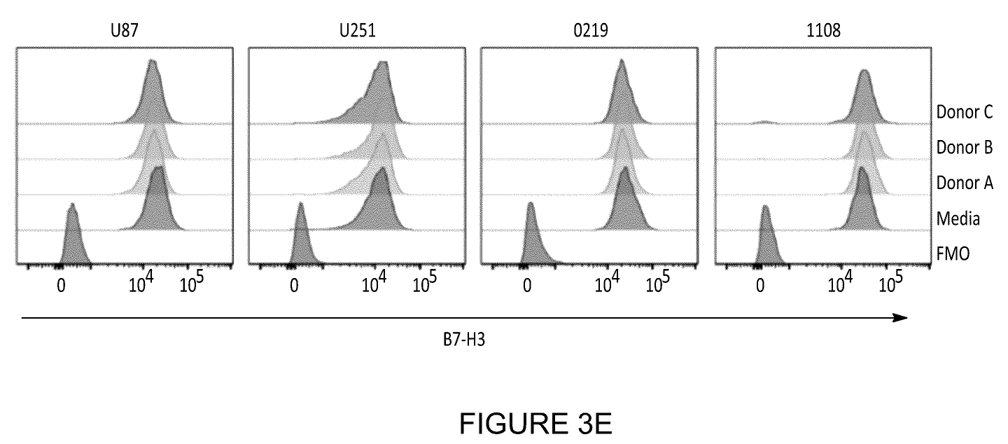

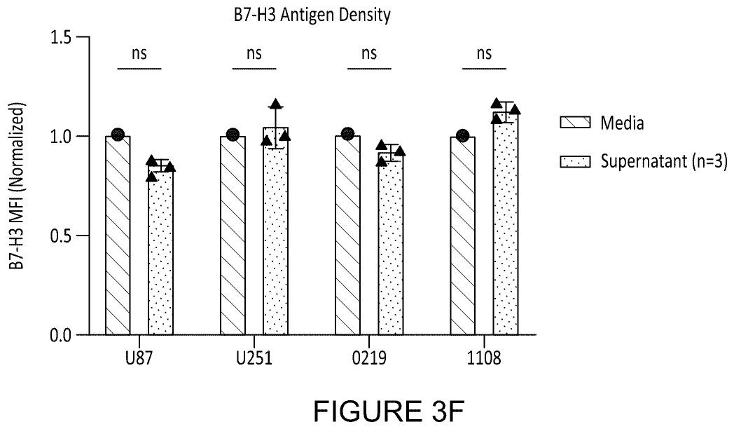

Glioblastoma (GBM) is the most common form of adult brain cancer with a median survival time of approx. 16 months. Currently, standard therapy includes resection, radiation, and use of chemotherapy (e.g., alkylating agents). The presently disclosed subject matter analyzed the expression levels of B7-H3 in GMB cell models U87MG and U251 and in primary cells. As seen in FIGS. 1 and 2, B7-H3 was significantly expressed in U87MG and U251 cells as well as in six (6) independent primary GMB cell lines (identified by codes 2019, 0507, 0604, 1108/1018, 2021-08-09, and/or 2021-06-22). Next, it was determined whether the expression of B7-H3 was modulated in GMB cell models upon incubation with conditioned medium from activated T cells. As seen in FIGS. 3A-3F, conditioned medium from activated T cells did not increase the expression levels of B7-H3 in all tested models.

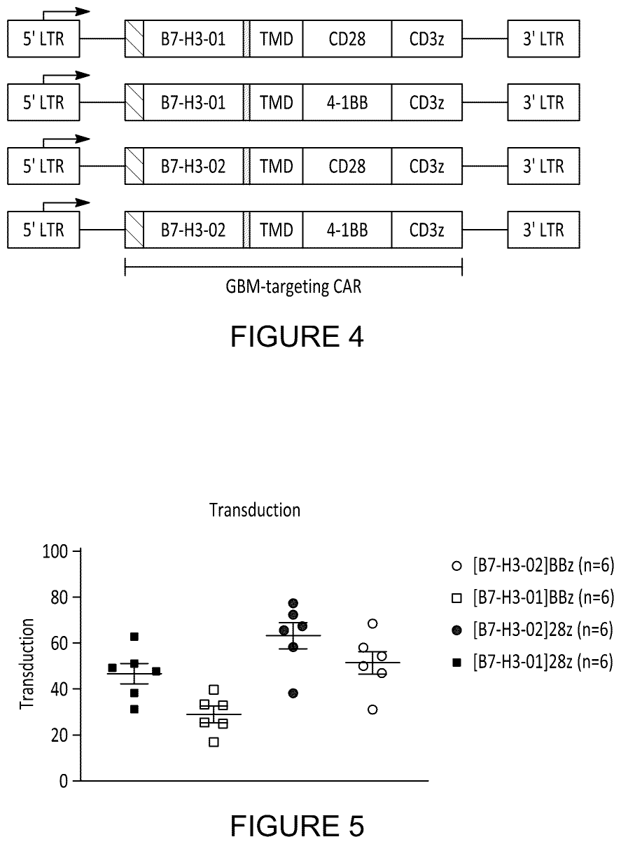

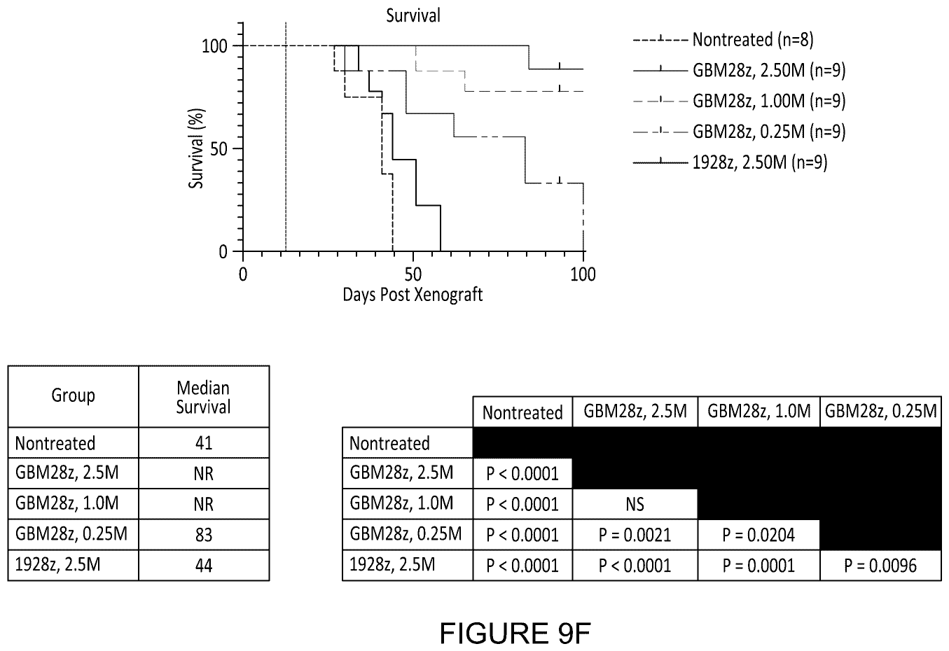

Given the role of B7-H3 in GMB, four (4) B7-H3-targeted CARs disclosed herein were generated: M3028z, M30BBz, MGA27128z, and MGA271BBz (FIG. 4). T cells transduced with the B7-H3-targeted CARs disclosed herein showed stable and efficient expression levels of the CARs and were able to induce in vitro cytotoxicity in U87MG and U251 cell lines (FIGS. 5-7C). Notably, B7-H3-targeted CAR T cells secreted proinflammatory cytokines upon exposure to antigen-positive cancer cell lines (FIG. 8).

Currently, use of immunotherapeutics such as antibodies and cell therapy has been unsuccessful for treating glioblastoma. Thus, the presently disclosed cells were tested in in vivo setting to determine their efficacy against glioblastoma. Briefly, orthotopic models of GBM were created by implanting U251 GMB cell lines in the central nervous system (CNS) of the animal, which were then treated with the presently disclosed cells (FIGS. 9A and 9B). As shown in FIGS. 9C and 9D, GBM28z cells conferred survival advantage through reduction of tumor burden. Further, GBM28z cells has a dose-dependent response and conferred a survival advantage in a dose dependent manner (FIGS. 9E, 9F, 9H, and 9I). Notably, GBM28z cells did not elicit toxicities (FIGS. 9G and 9J). Overall, these data show that GBM28z cells are efficient and resilient within the CNS and are safe.

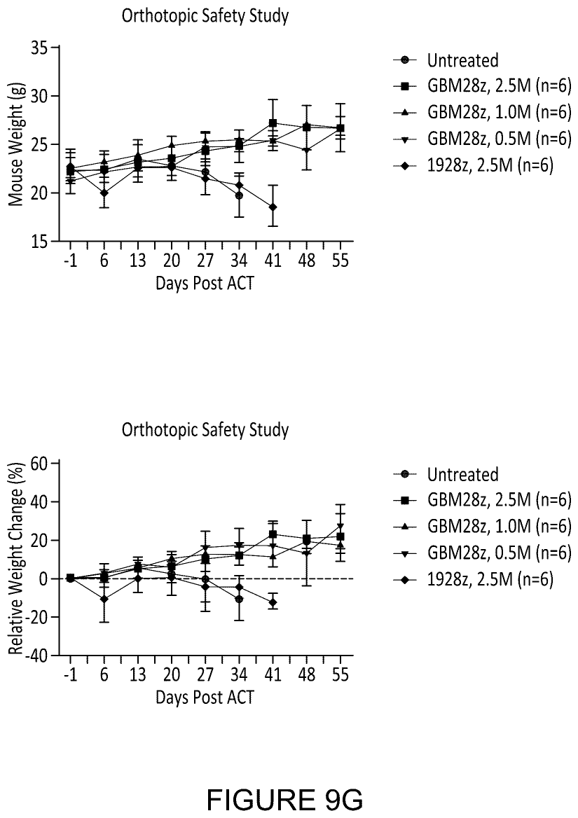

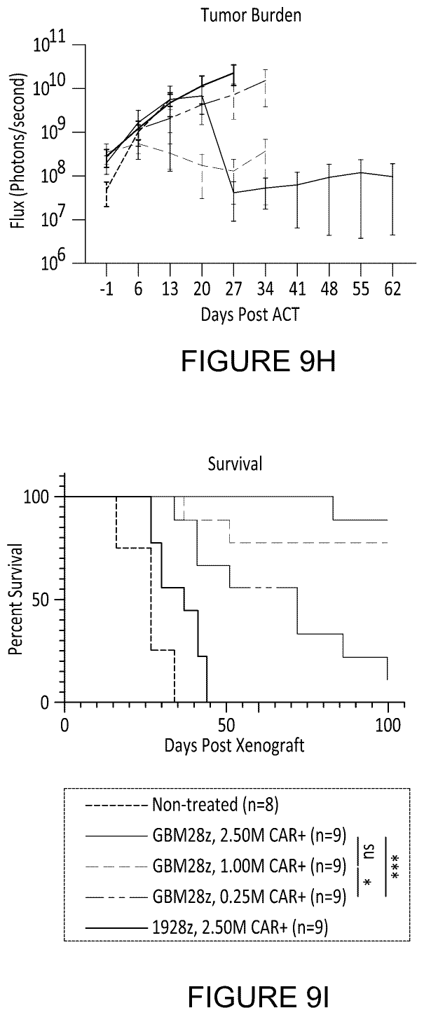

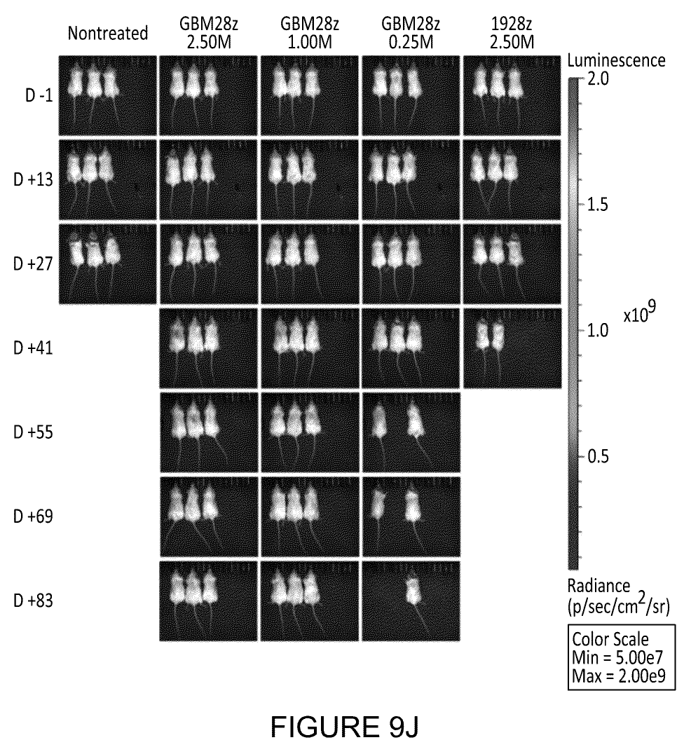

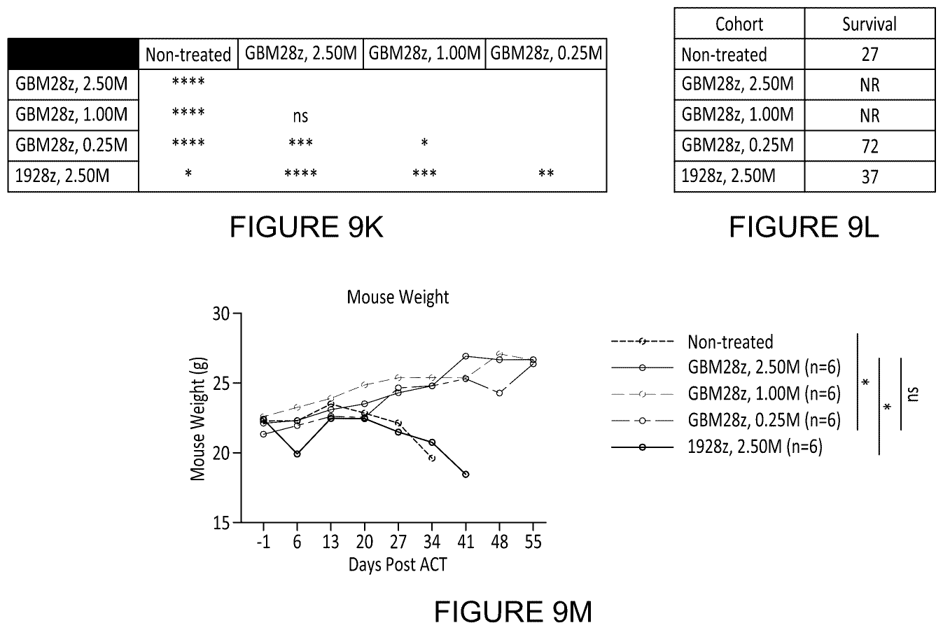

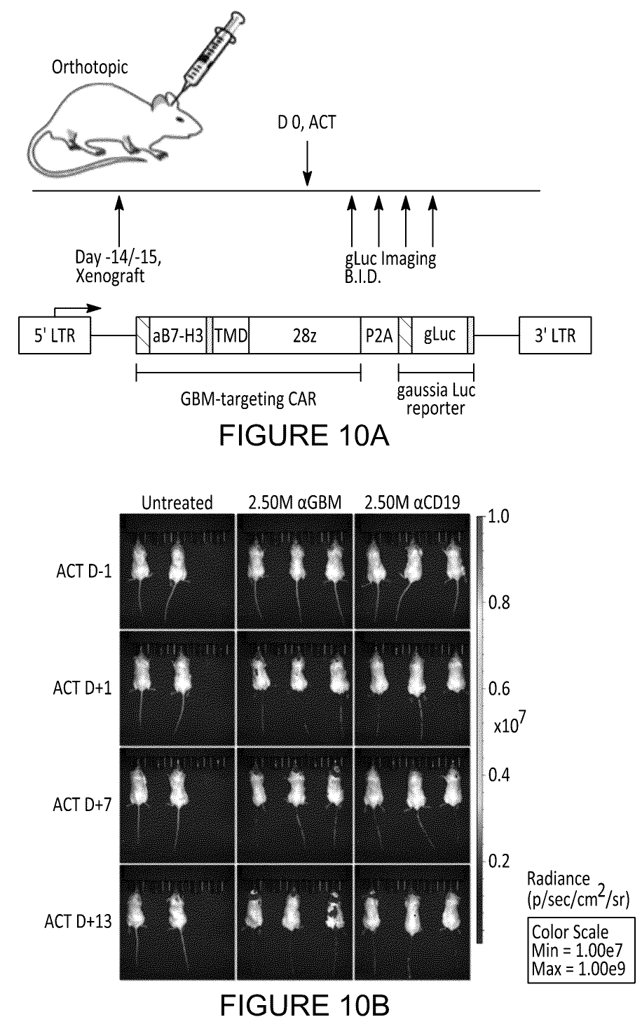

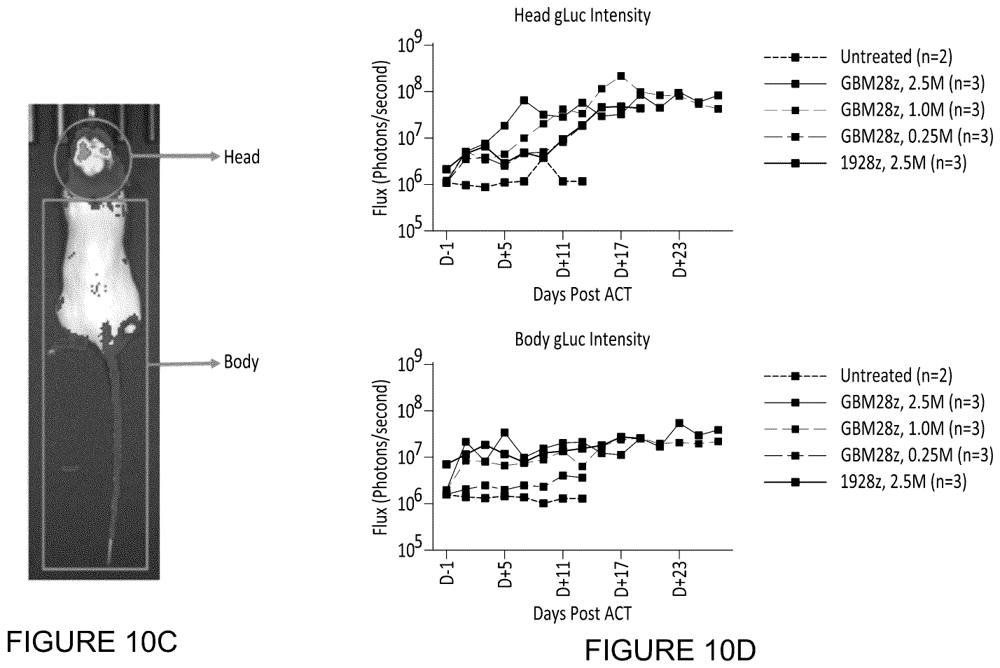

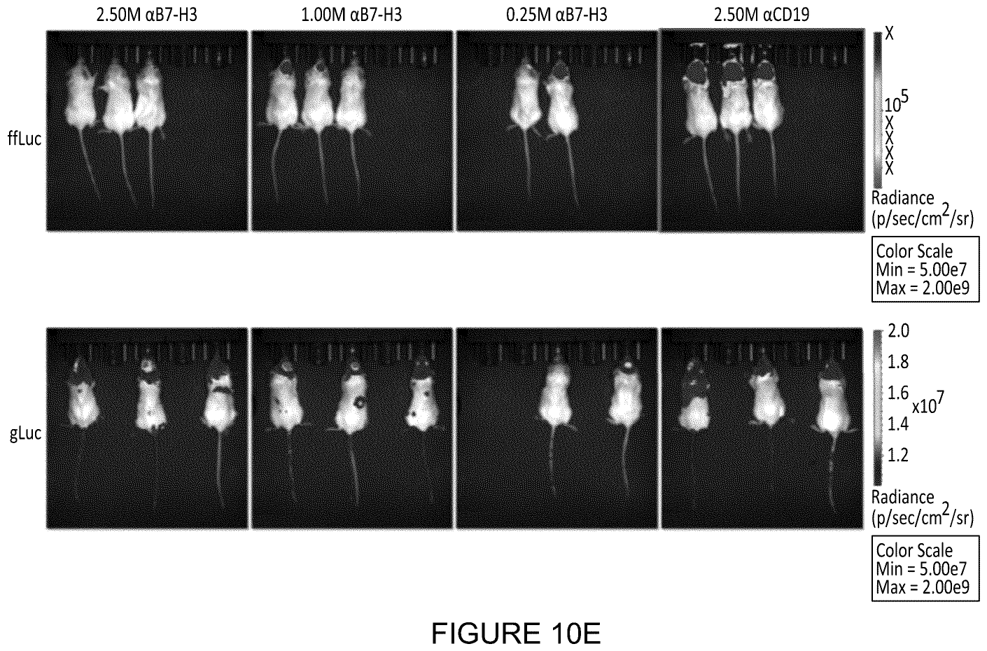

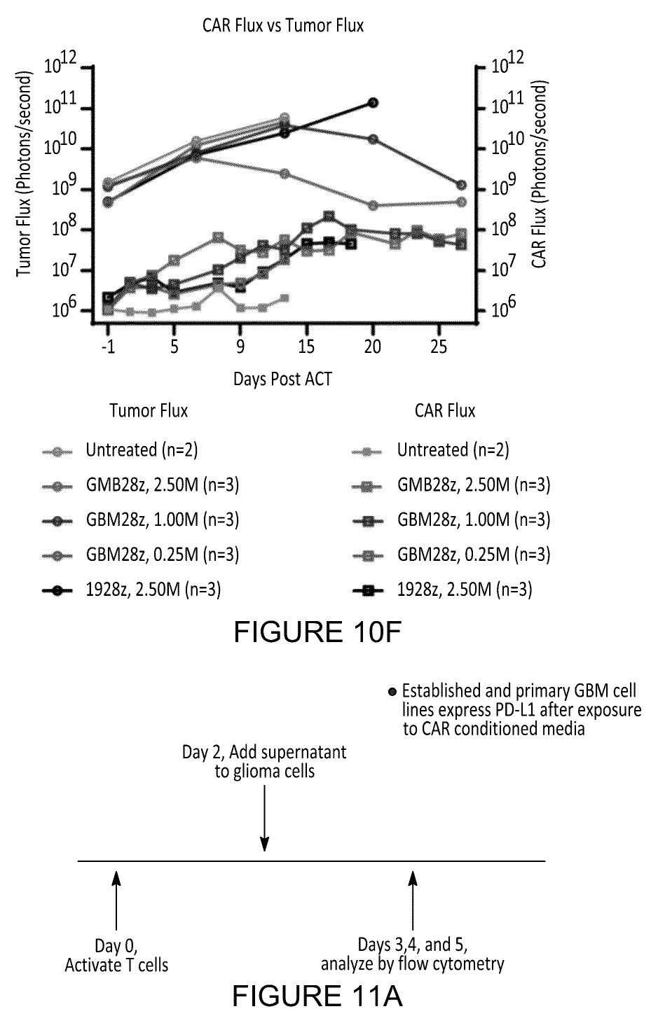

Next, it was determined whether the presently disclosed cells could traffic to the disease site (e.g., CNS). Using GMB28z cells expressing a reporter, it was observed that GBM28z cells trafficked to the intracranial disease site and were also able to expand at the tumor location (FIGS. 10A-10D). Importantly, it was also observed that GBM28z expanded and persisted at the intracranial disease site, which correlated with tumor burden (FIGS. 10E and 10F).

Overall, B7-H3 represent a valid target in glioblastoma models which can be successfully targeted by the presently disclosed cell.

### Example 2

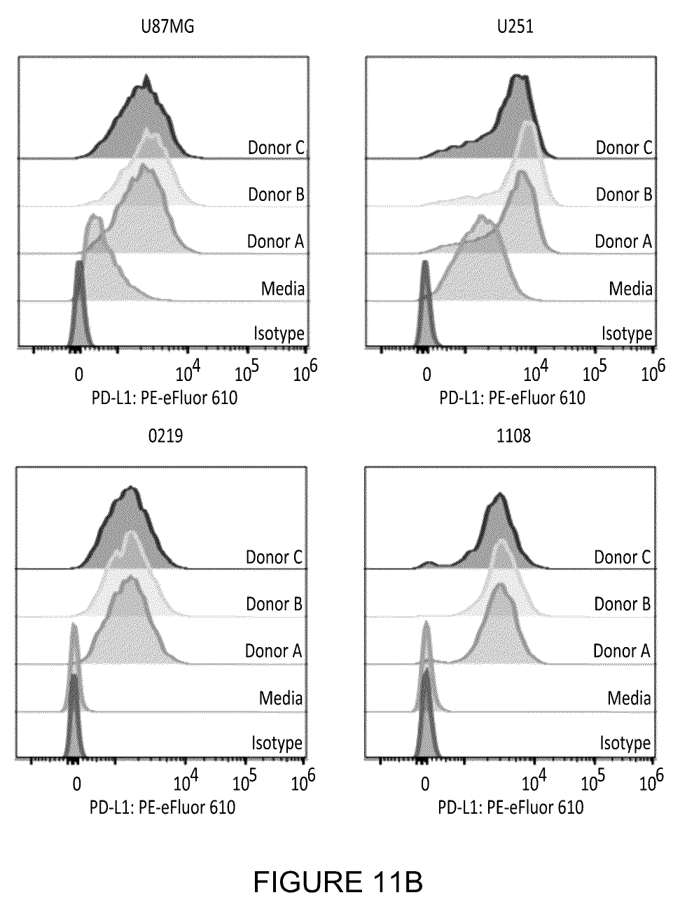

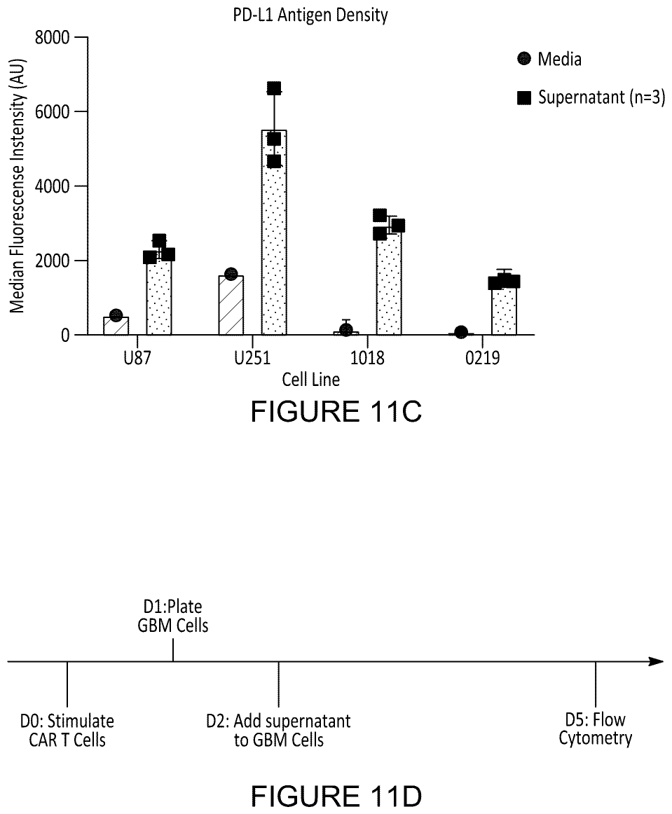

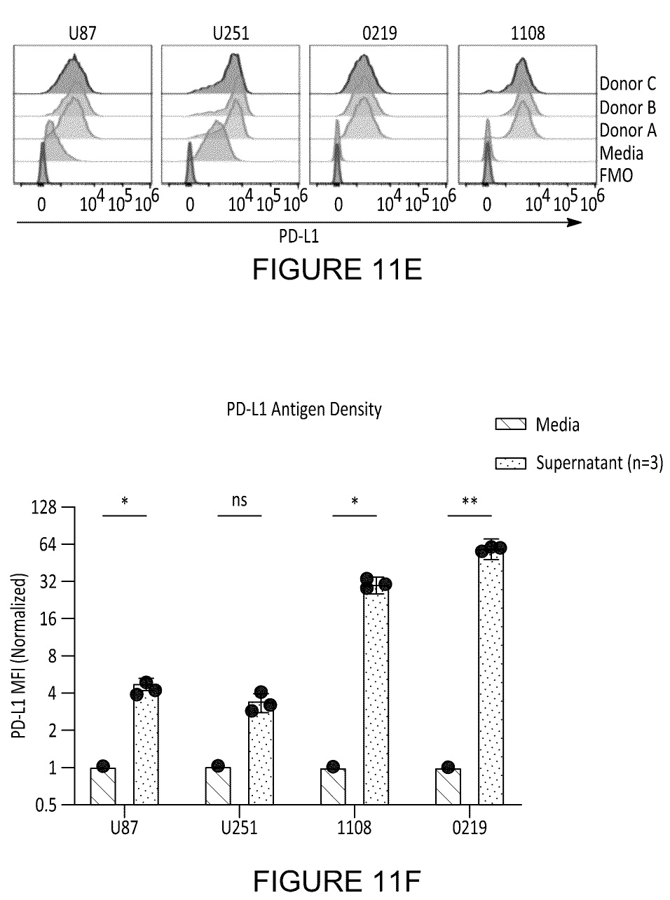

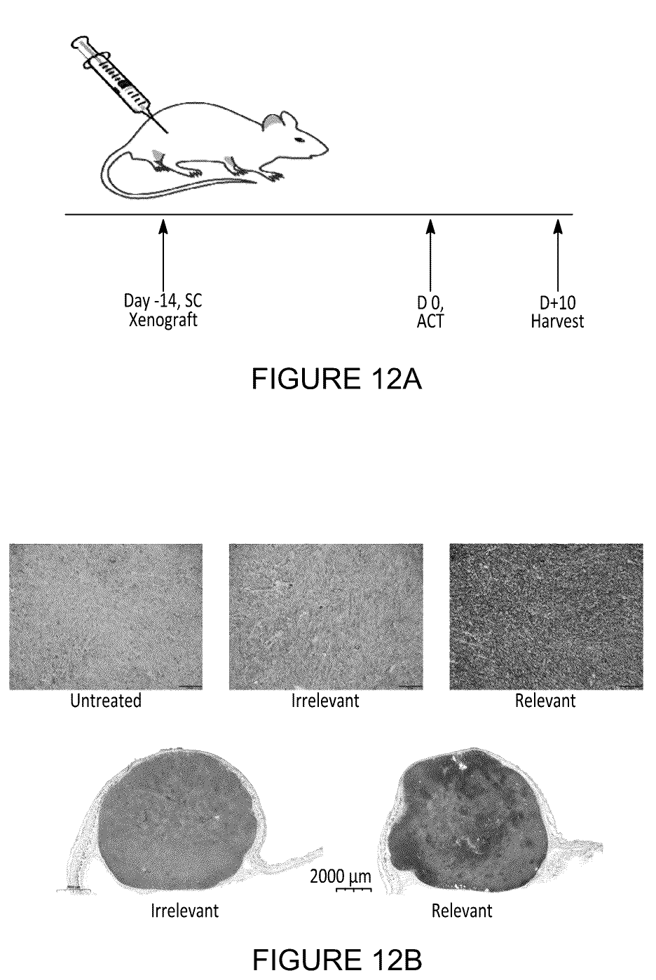

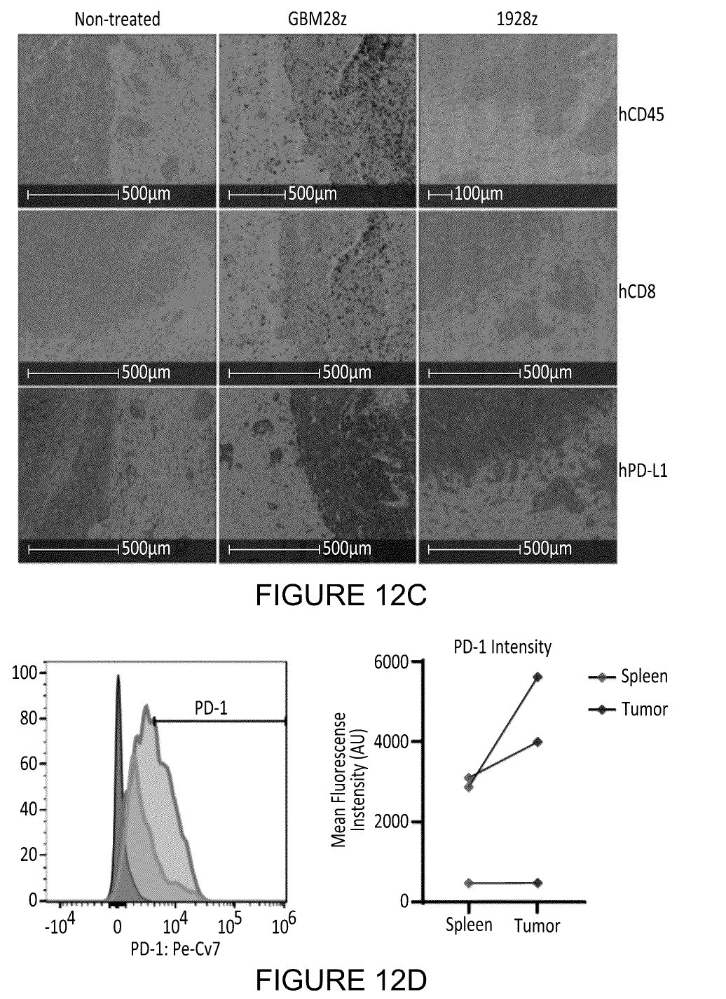

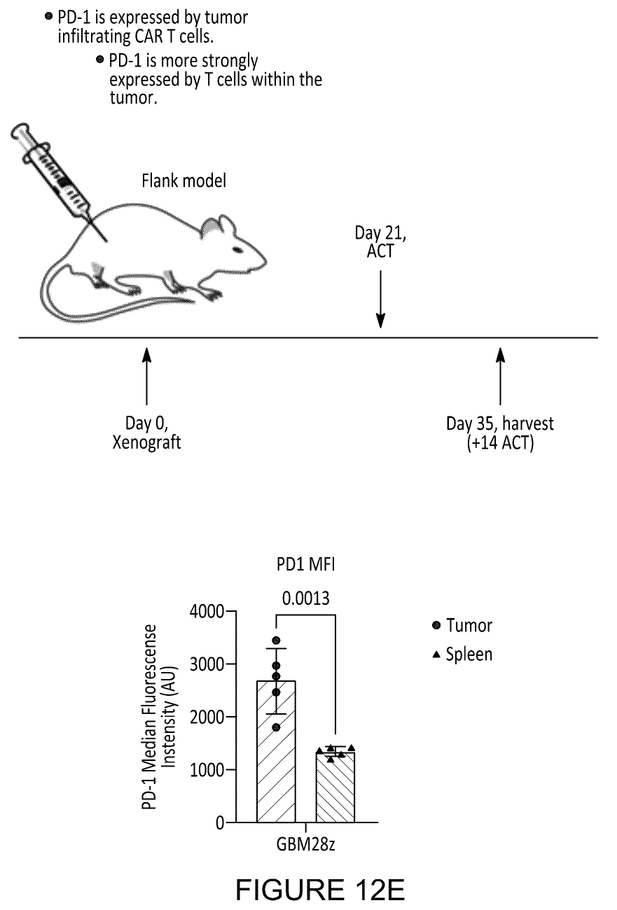

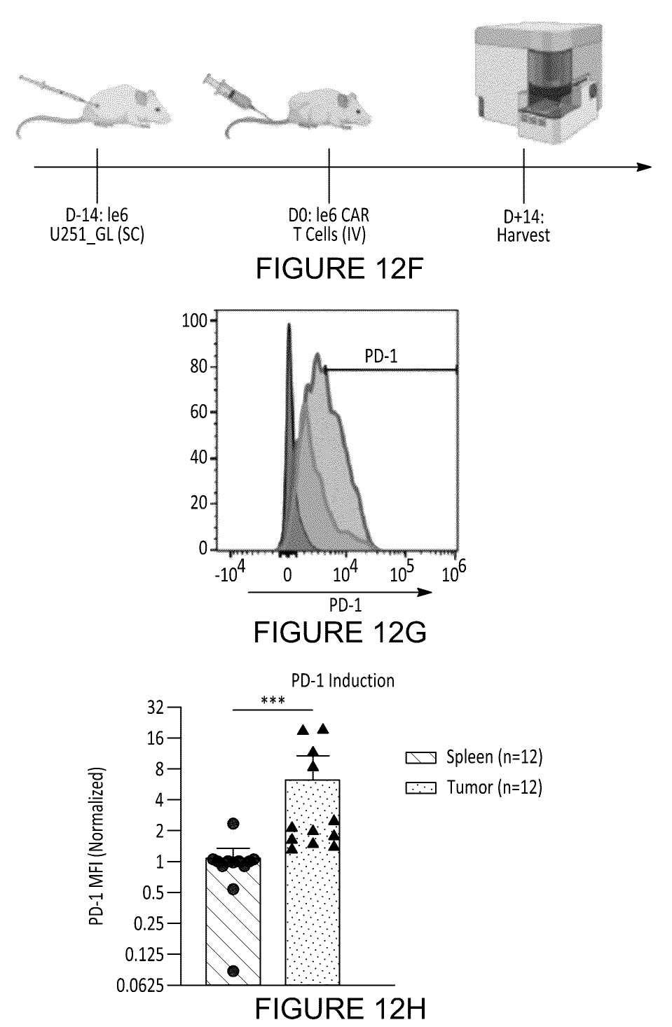

One of the major drawbacks of application of cell therapies for solid tumors is the presence of an immunosuppressive tumor microenvironment. In order to overcome this possible limitation, the presently disclosed subject matter tested whether treatment of glioma tumor with the presently disclosed cells increased expression of inhibitory checkpoints. For this purpose, as shown in FIGS. 3A and 3D, B7-H3 tested cell models were incubated with conditioned medium from activated T cells. It was observed that established and primary GBM cell lines expressed PD-L1 after exposure to conditioned media (FIGS. 11A-11C). Next, it was determined whether these observations could be made in in vivo as well. As shown in FIGS. 12A-12C, PD-L1 was strongly upregulated in response to treatment with the presently disclosed cells (e.g., GBM28z cells). Further, it was observed that PD-1 was strongly expressed by T cells within the tumor (FIGS. 12D-12G). These data indicate that GBM is characterize by in vivo adaptive resistance.

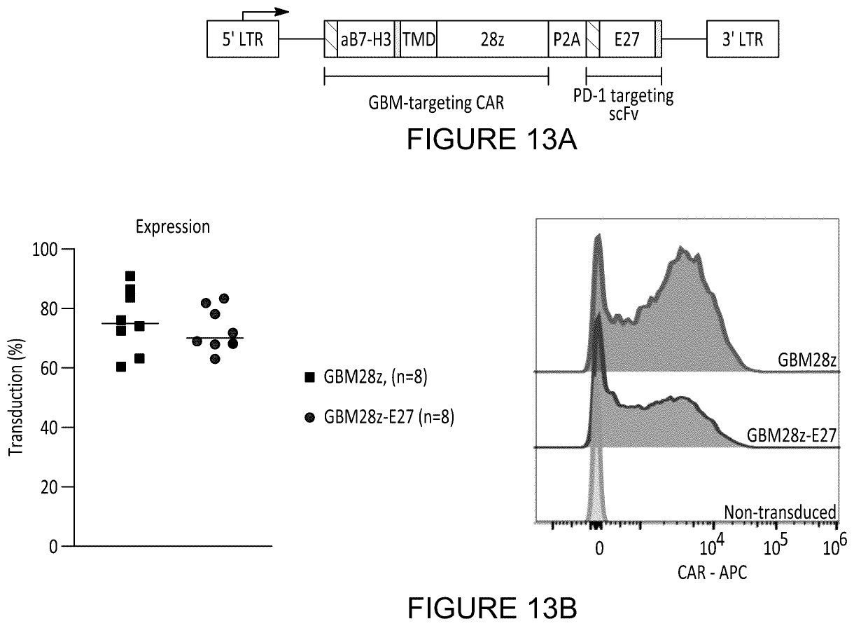

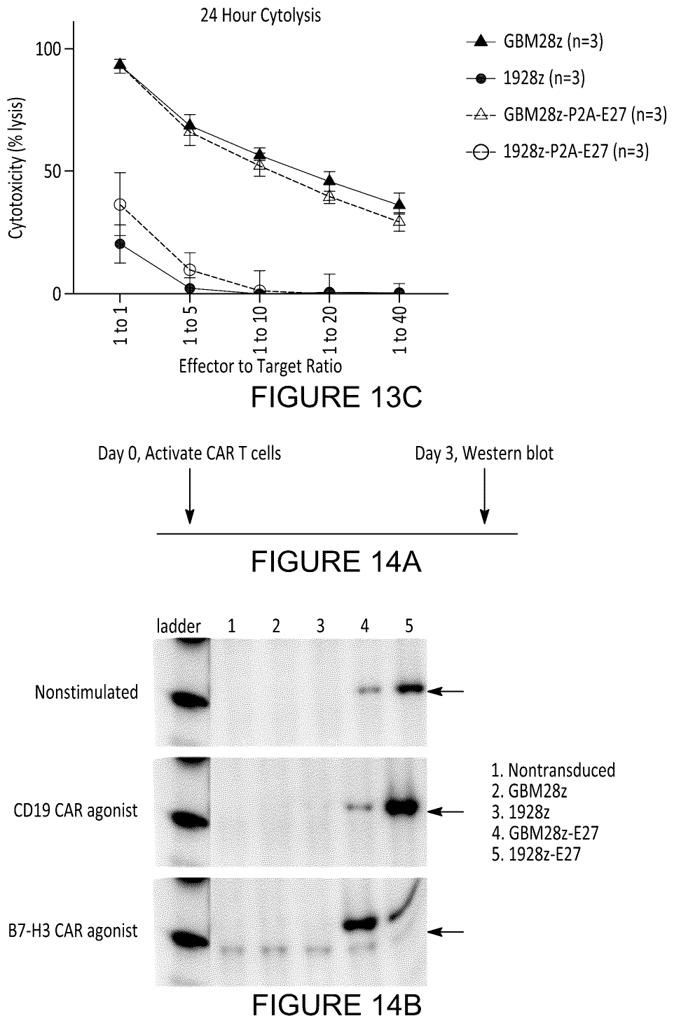

It was hypothesized that delivery of a soluble scFv that blocks PD-L1 or PD-1 could overcome the observed adaptive resistance. To that end, T cells transduced with the B7-H3-targeted CARs and a soluble scFv disclosed herein were generated (FIG. 13A). These cells showed high transduction and cytotoxic effects (FIGS. 13B and 13C) as well as high expression level of the E27, a soluble scFv that binds PD-1 (FIGS. 14A and 14B).

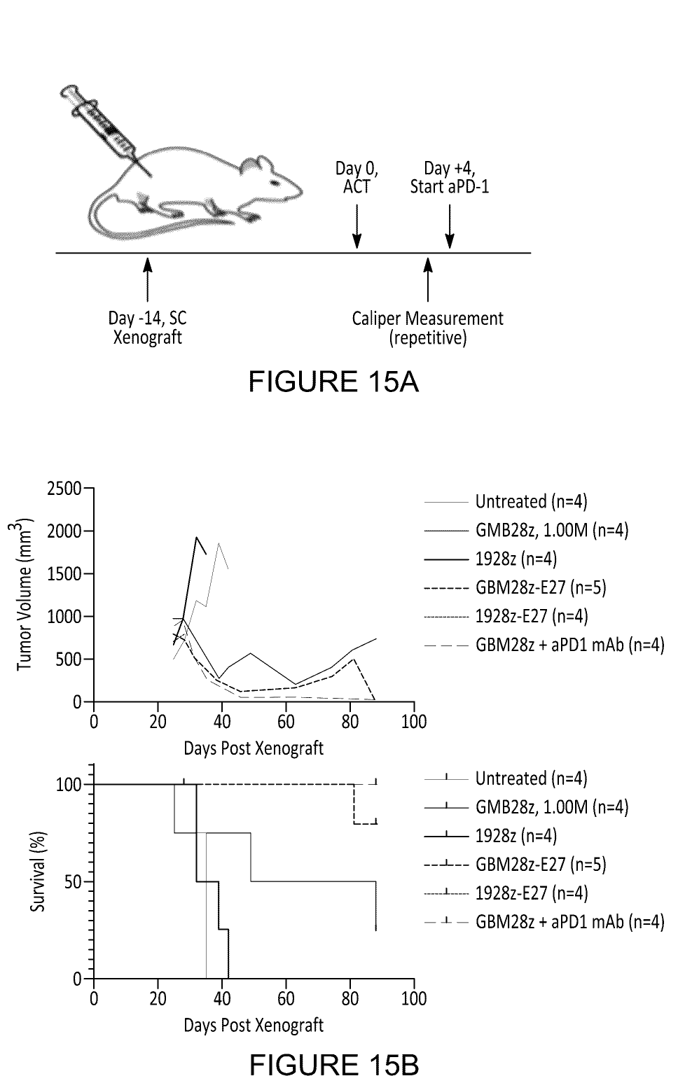

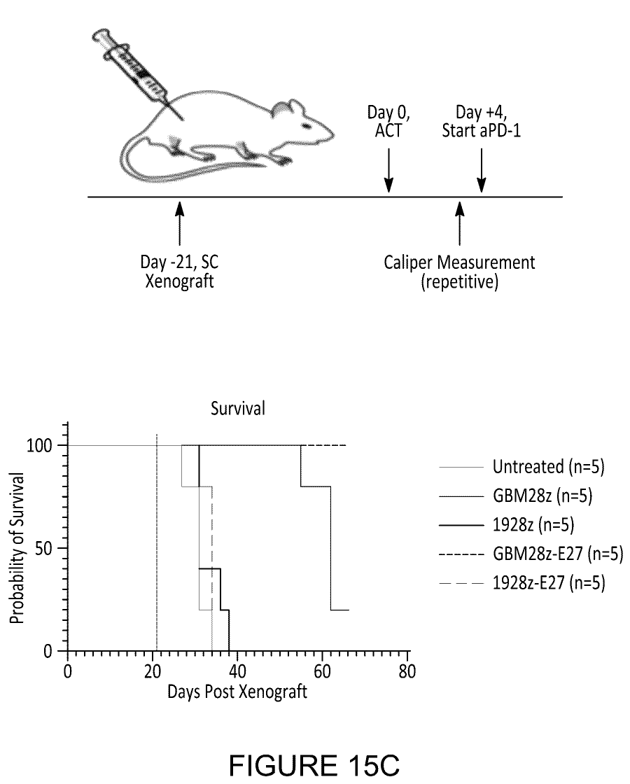

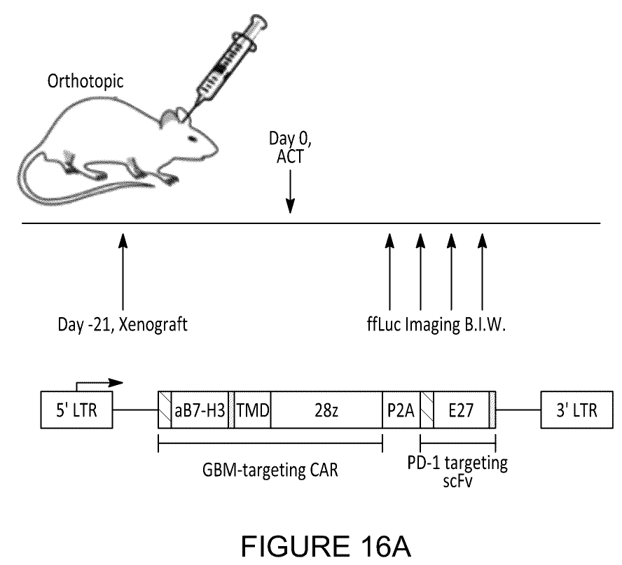

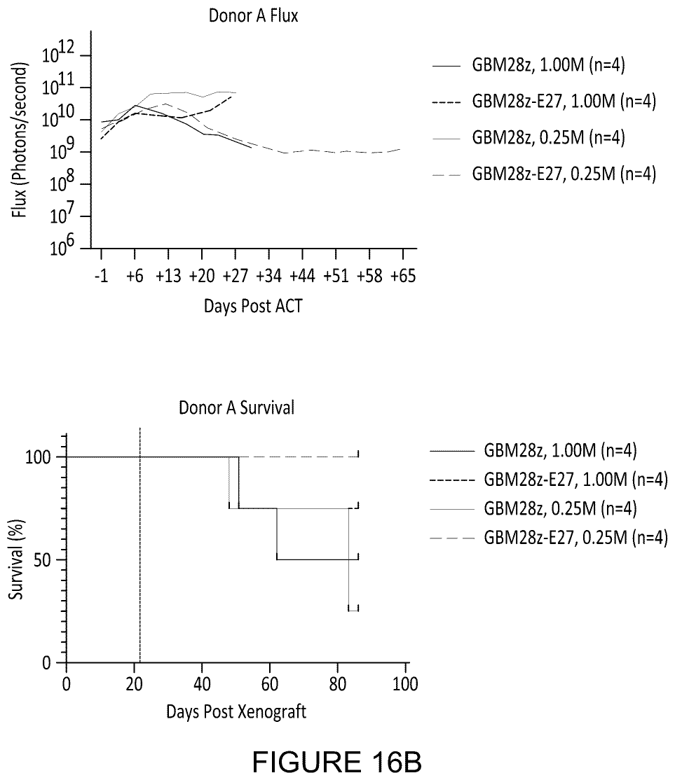

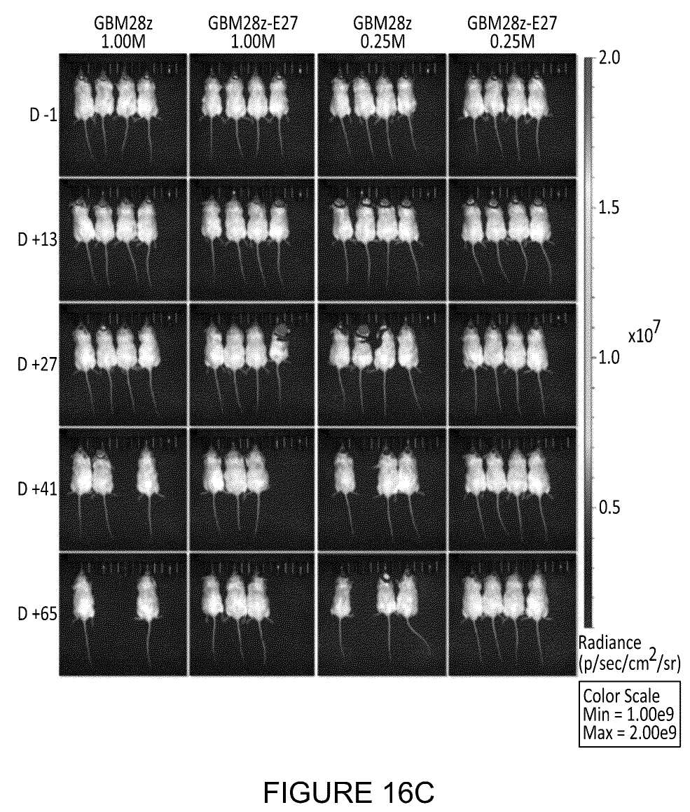

To determine whether PD-1 blockade could rescue the in vivo activity of the cells, xenograft model of glioblastoma was established (FIG. 15A). As shown in FIGS. 15B and 15C, GBM28z-E27 cells reduced tumor burden and increased survival, indicating that the PD-1 blockade rescued the activity of the cells on the tumor. Next, the effects of the presently disclosed cells (e.g., GBM28z-E27 cells) within the CNS were studied. FIGS. 16A-16C show the improved ability of the GBM28z-E27 to reduce tumor burden and increase survival even at lower doses (e.g., 0.25M).

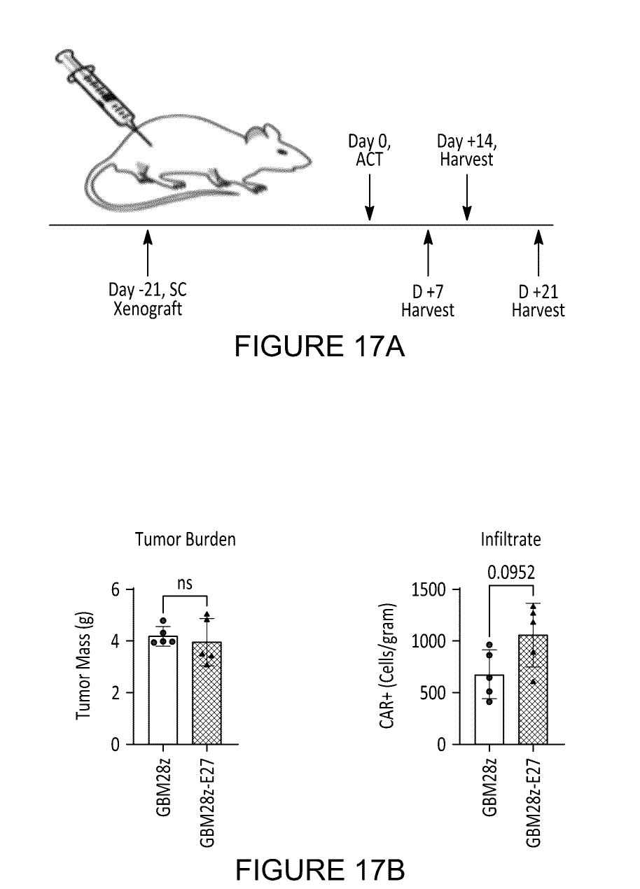

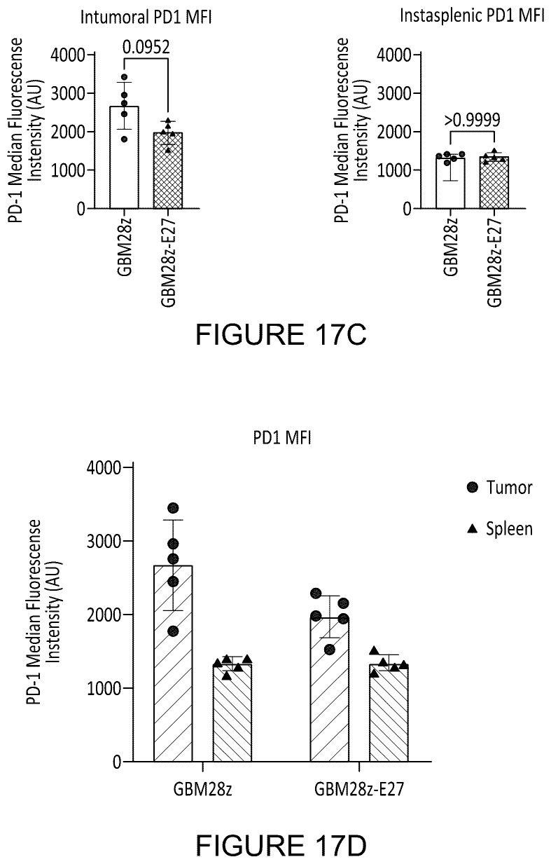

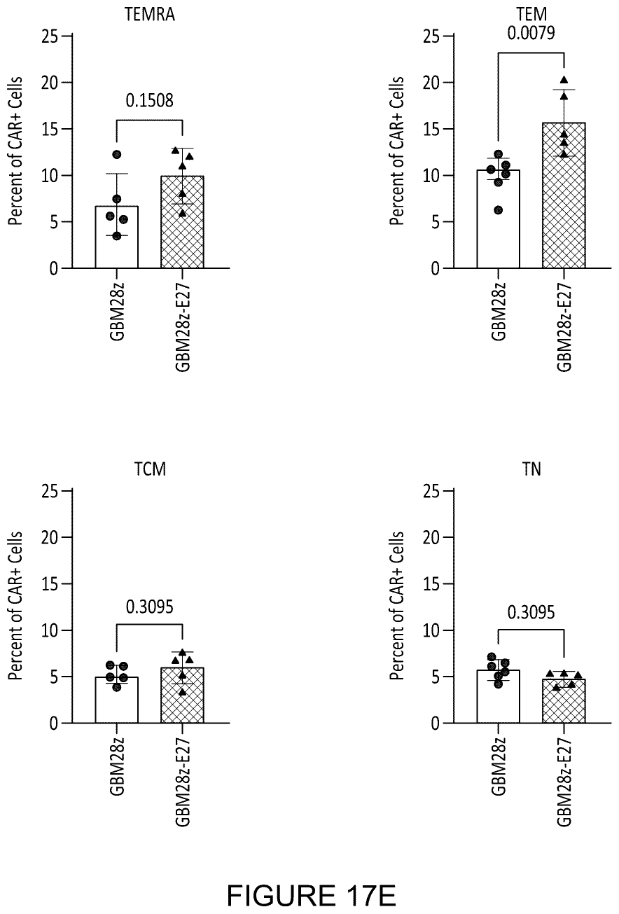

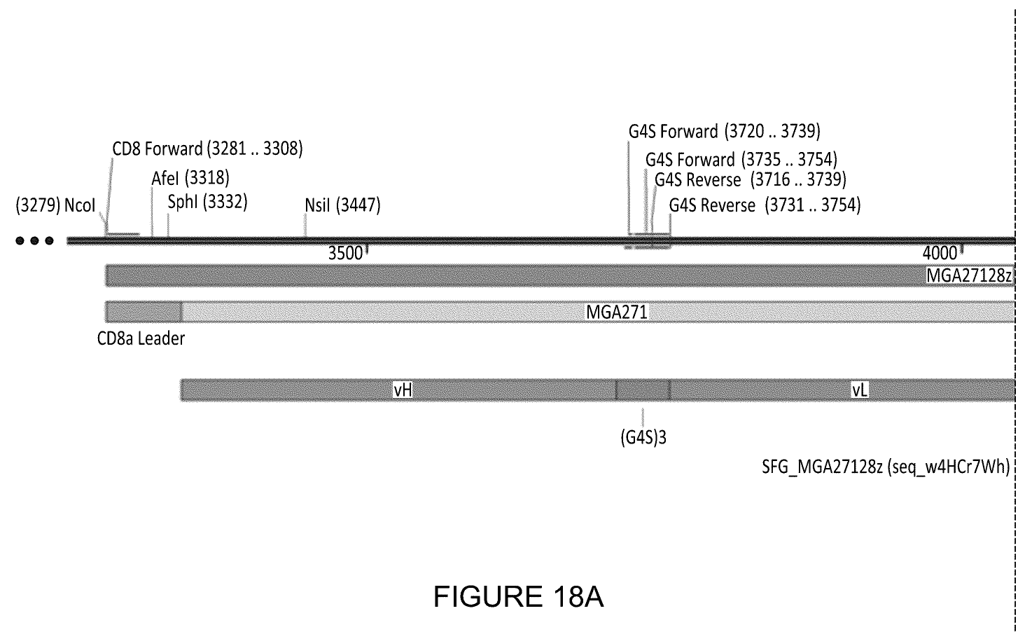

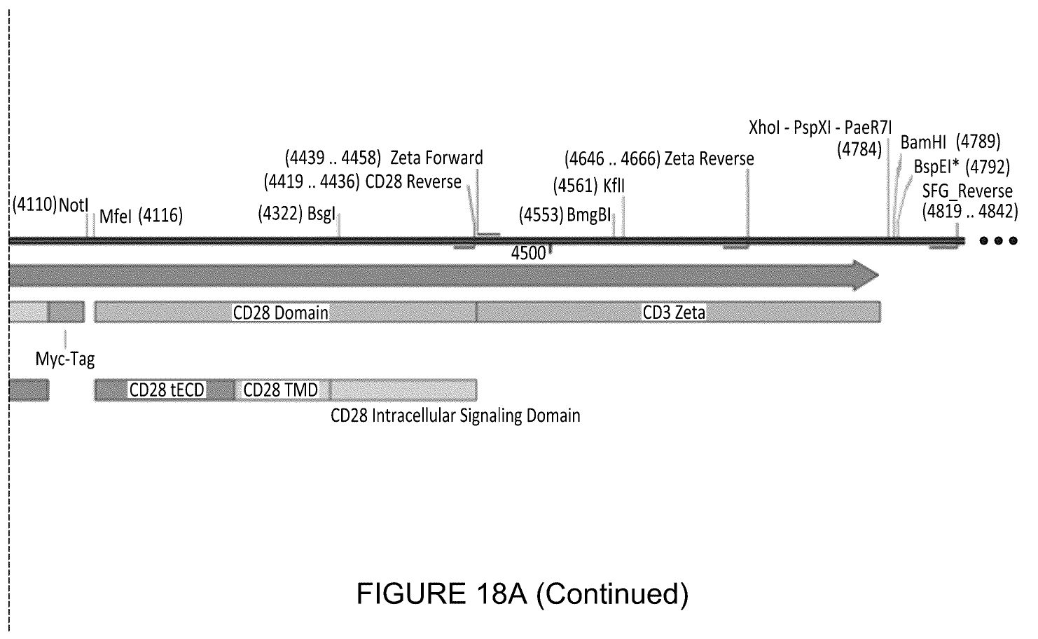

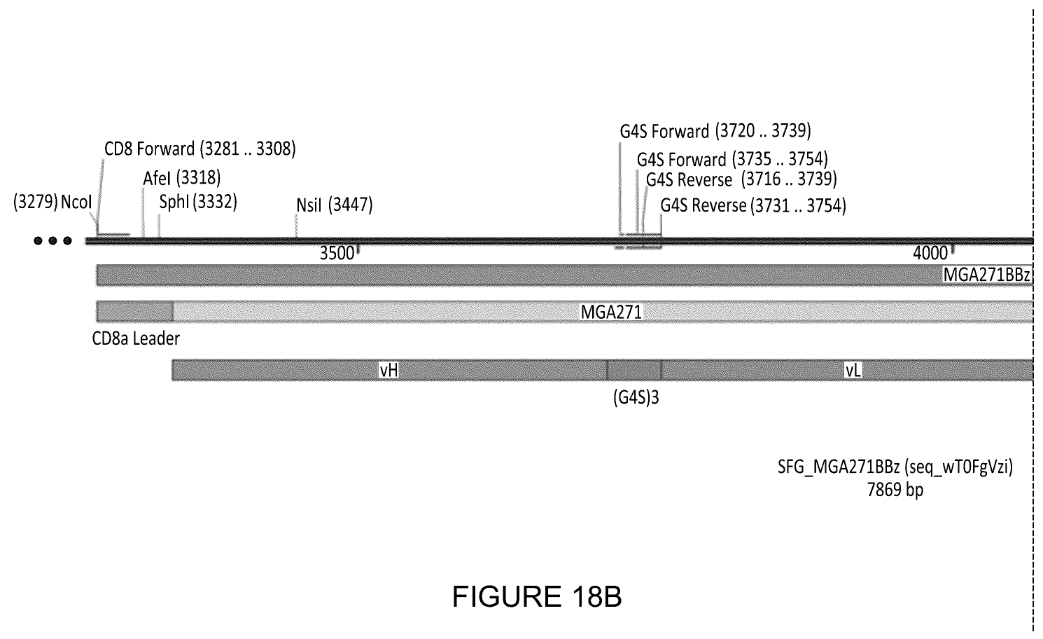

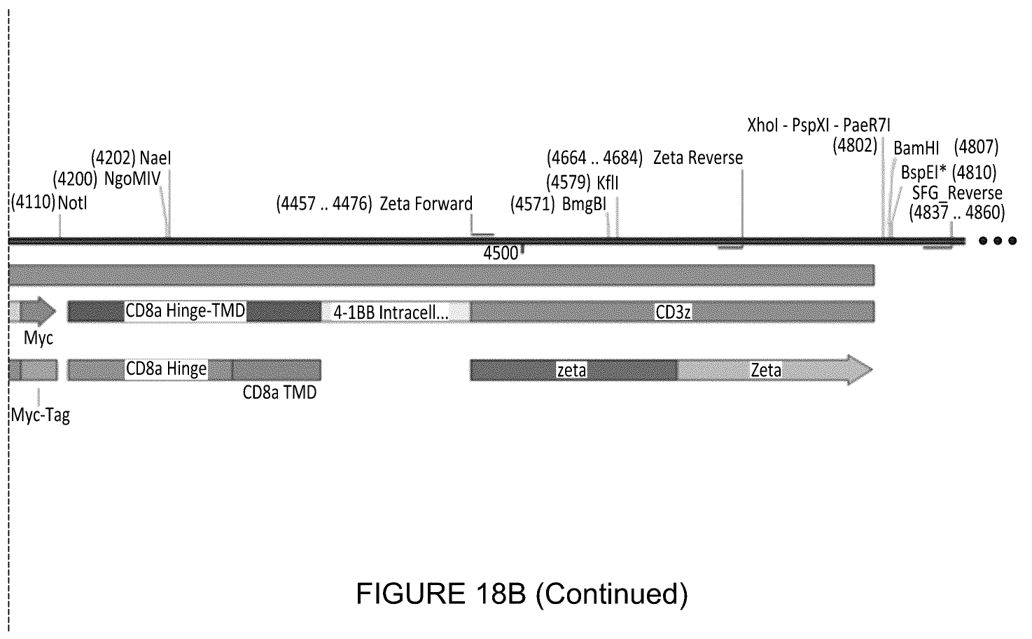

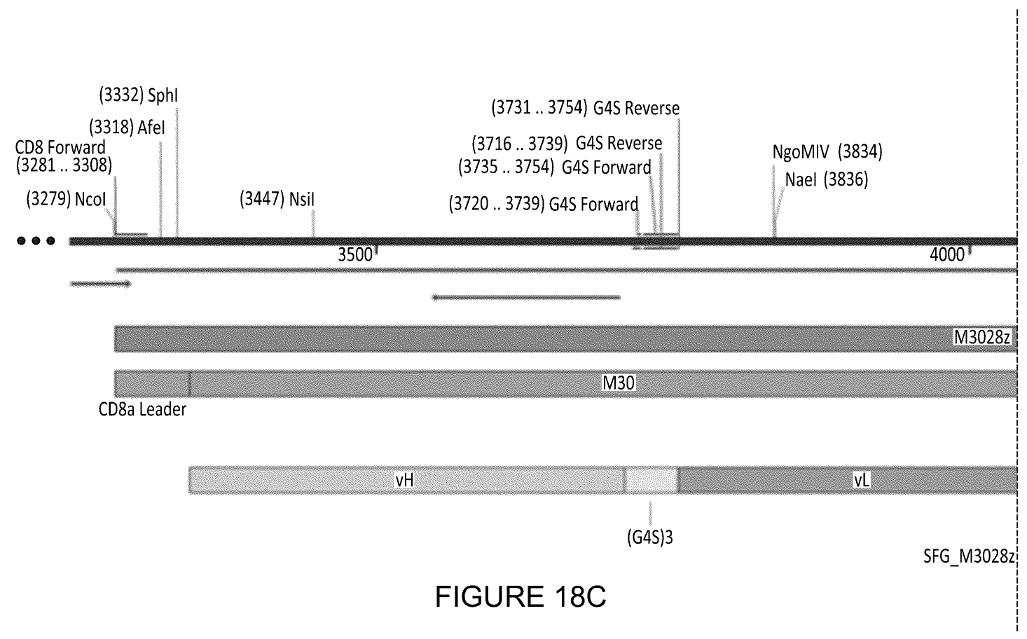

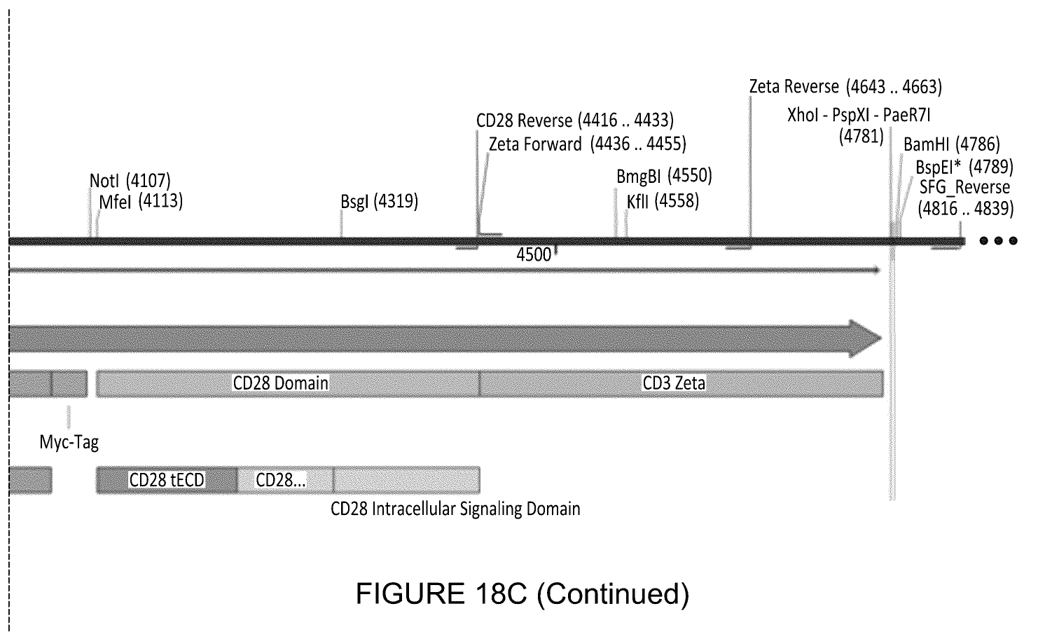

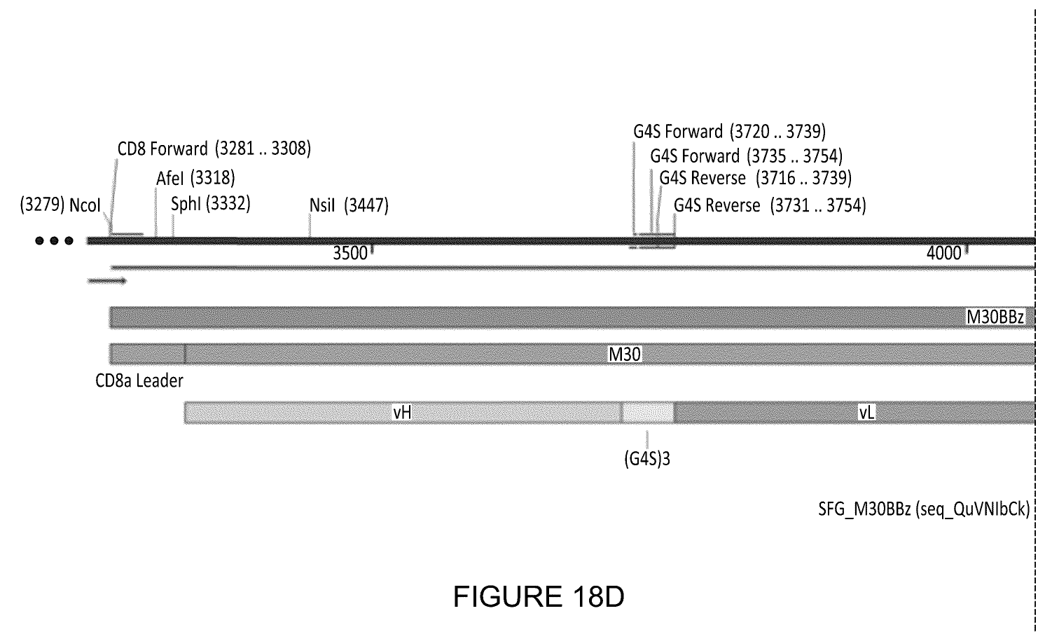

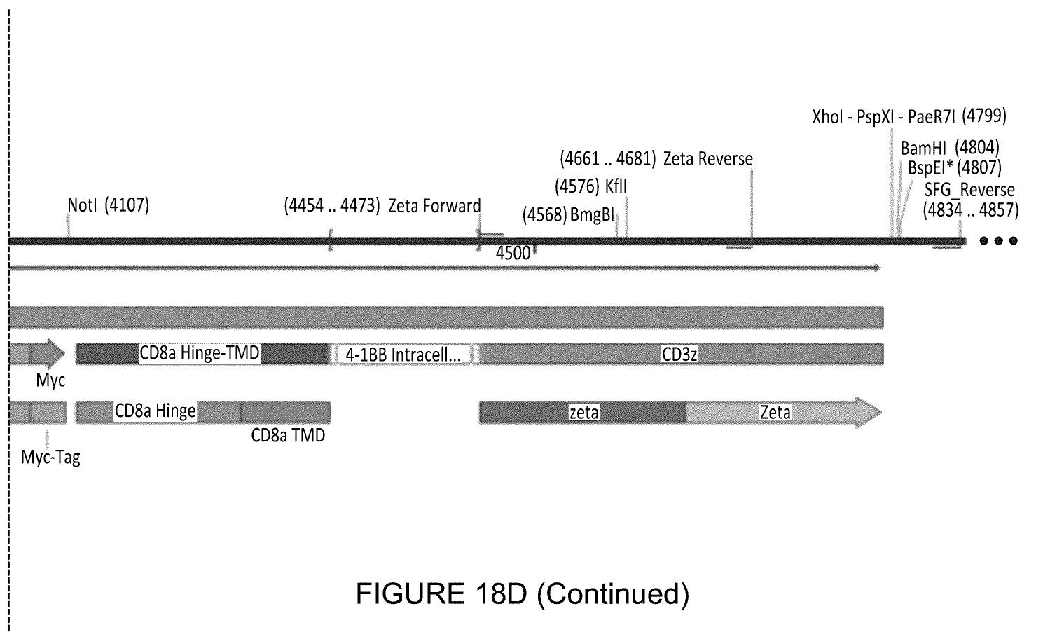

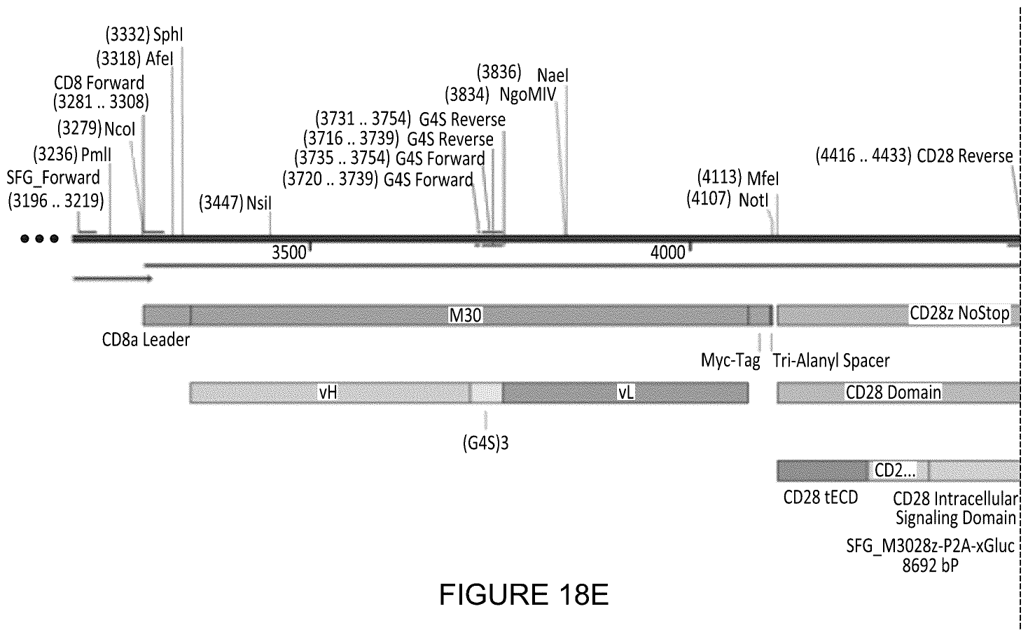

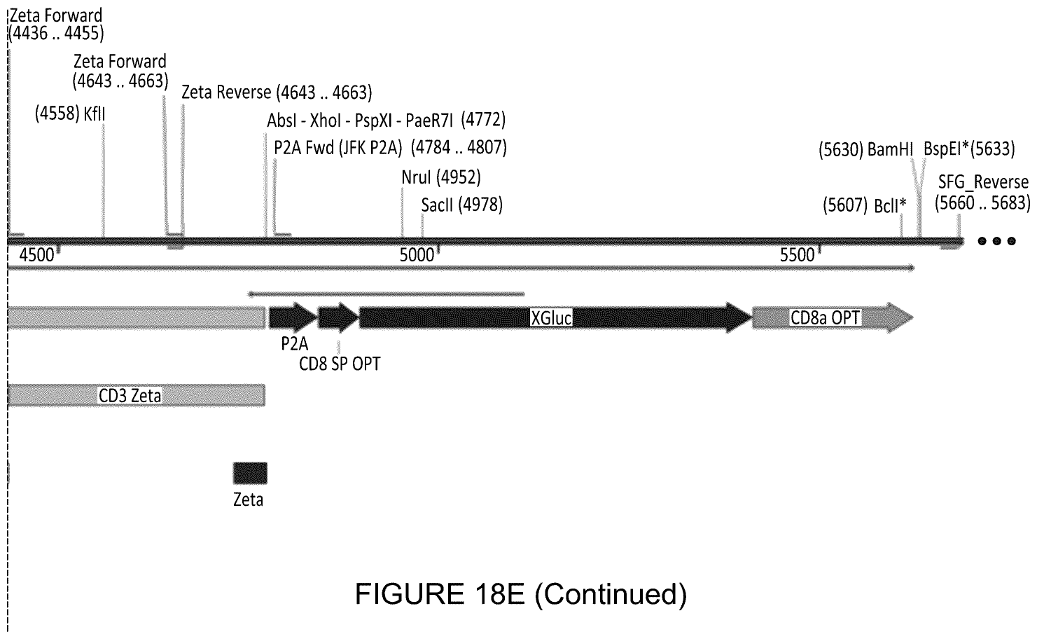

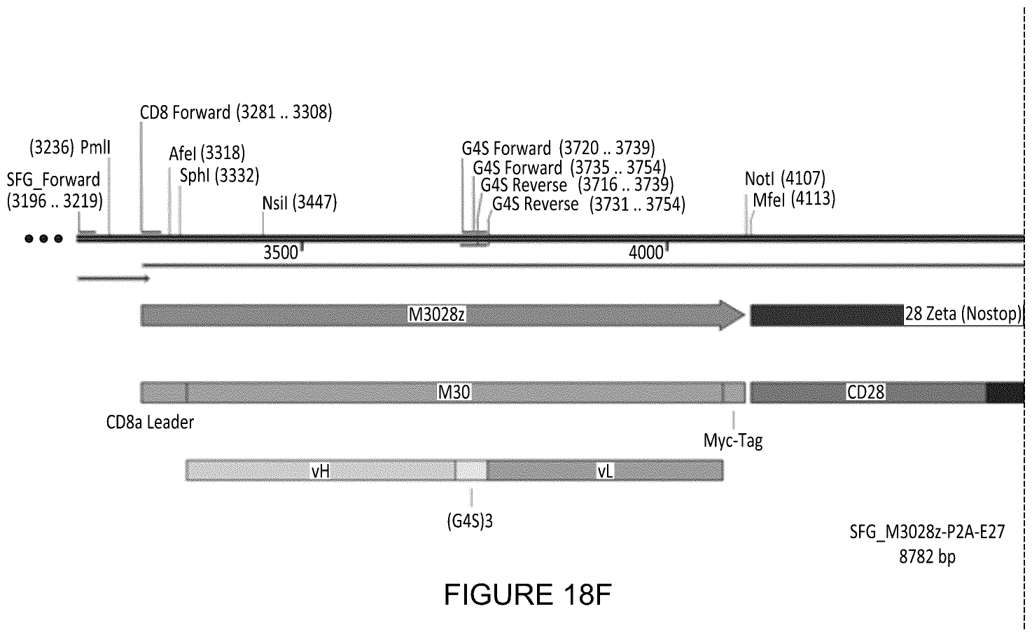

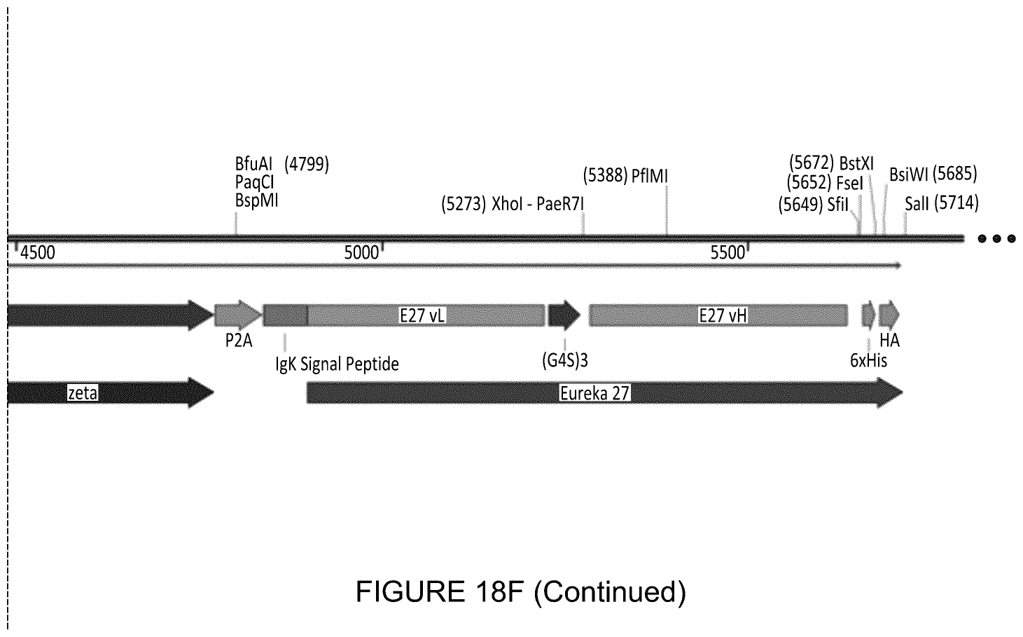

Finally, T cells infiltrated in the tumor were studied to determine mechanisms underlining the observed data. To this scope, animals were treated with GMB28z cells and GBM28z-E27 cells and cells were harvested at day 14 (FIG. 17A-17E). As seen in FIGS. 17A-17H, animals treated with GBM28z-E27 cells showed a higher number of infiltrated cells which expressed a reduced amount of PD-1. Importantly, intratumoral GBM28z-E27 cells were enriched with effector memory T cells and effector T cells, indicating persistence and resilience of these cells in this hostile GBM tumor microenvironment.

In conclusion, a PD-1/PD-L1 axis was identified as a GBM-specific mechanism of resistance to cell therapy which was overcome by the presently disclosed cells expressing a soluble scFv that binds to PD-1.

### Example 3

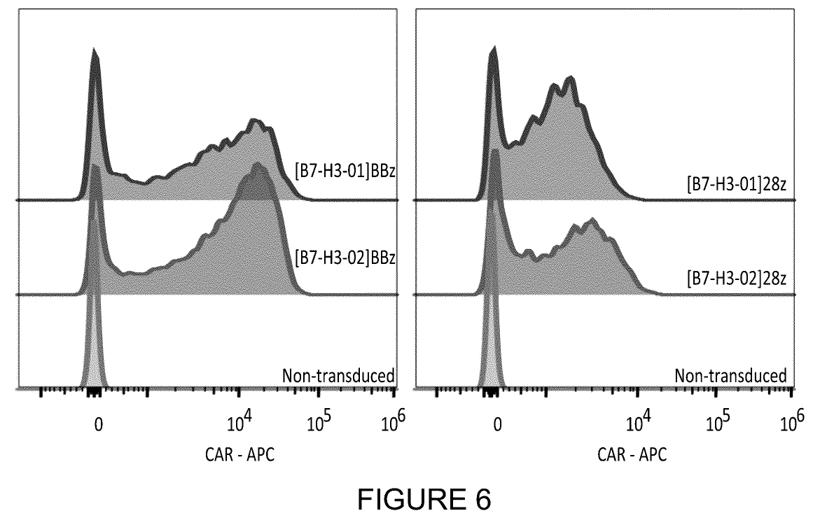

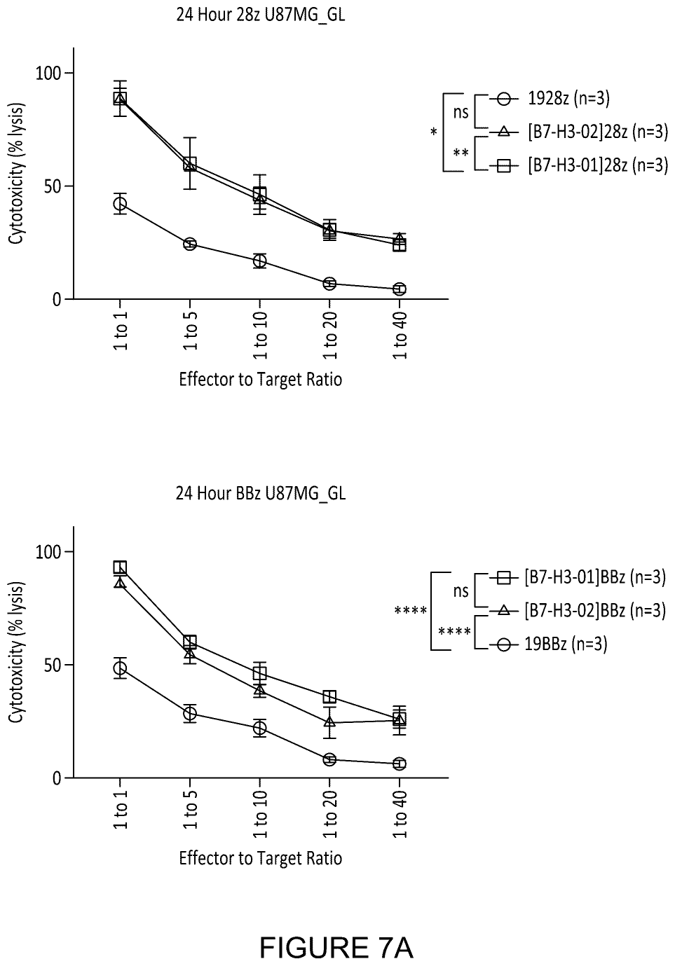

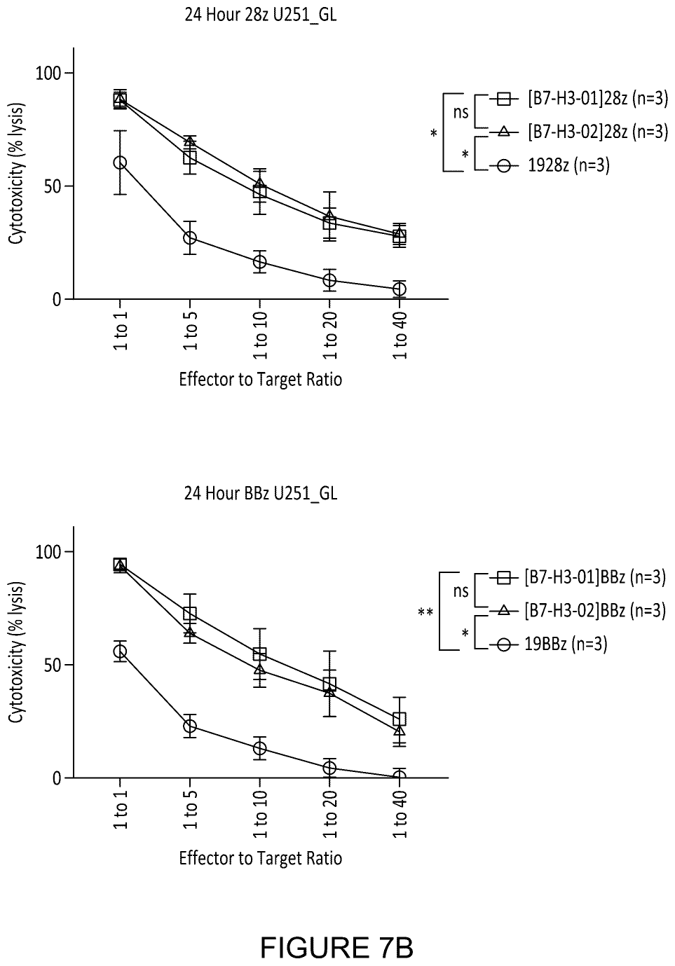

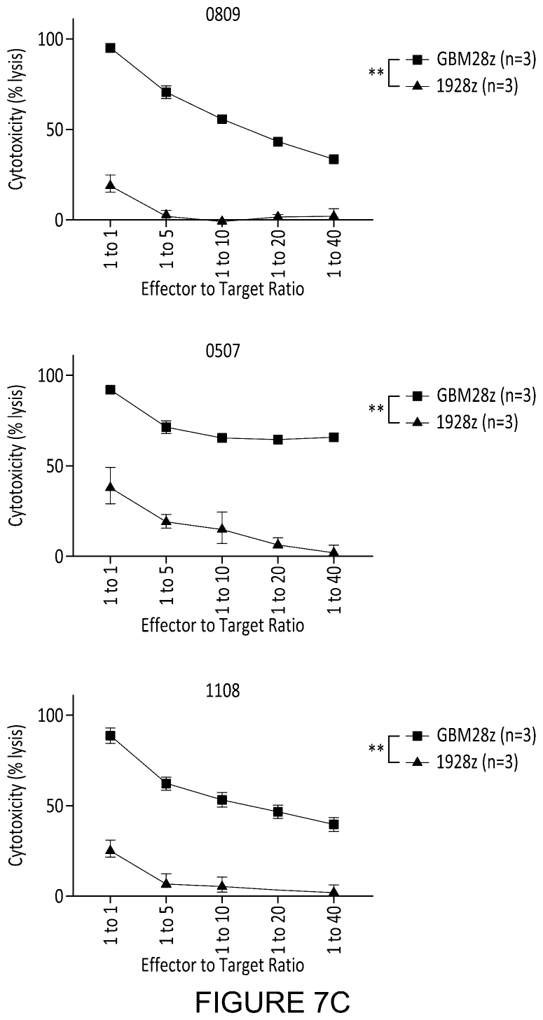

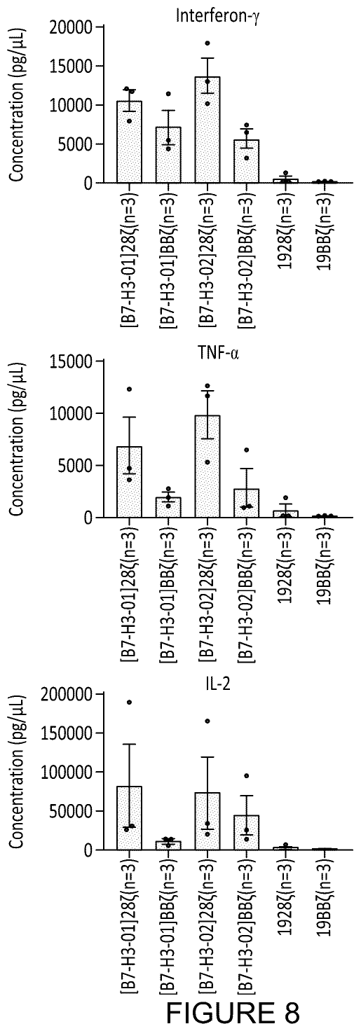

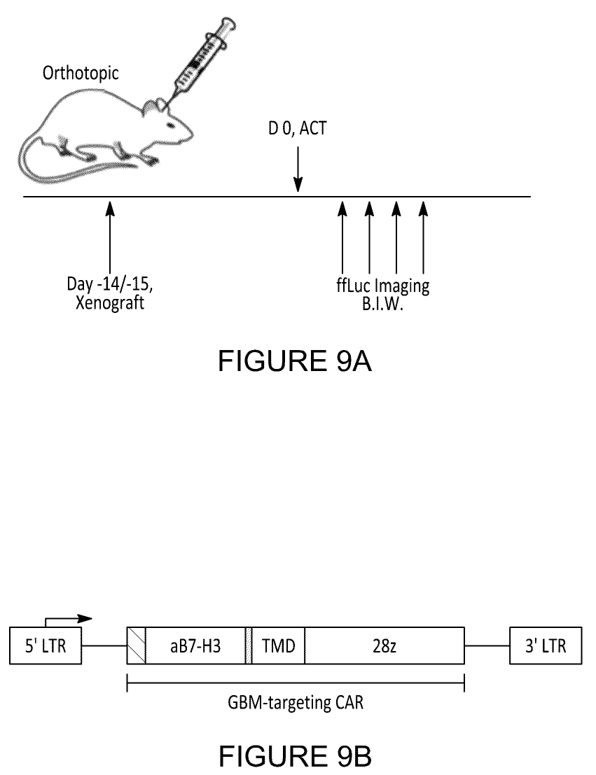

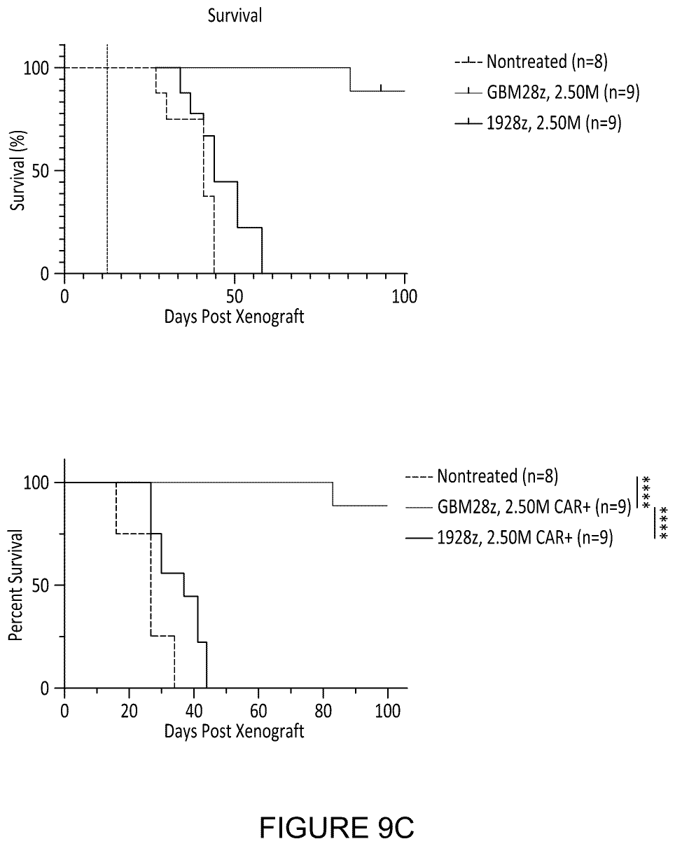

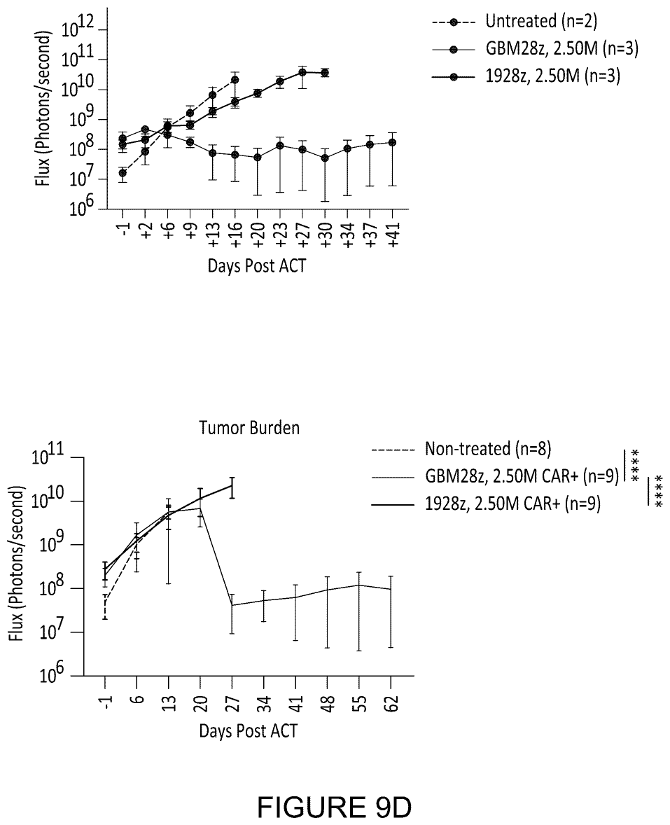

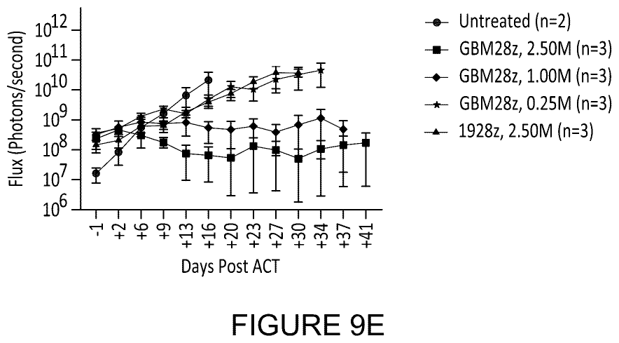

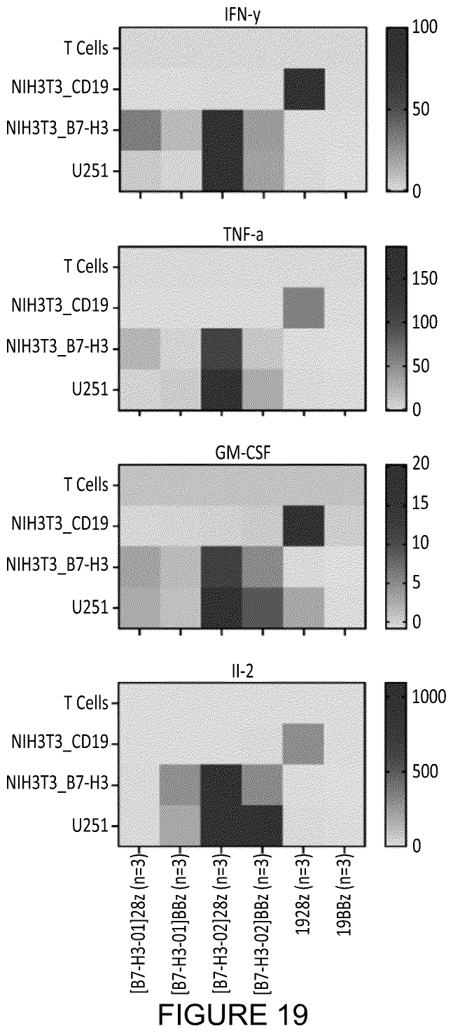

The presently disclosed subject matter first identified clinically validated B7-H3 targeting antibodies targeting different epitopes and constructed corresponding scFv fragments derived from heavy-light chain parings of these antibodies. Each scFv was marked with a c-myc tac to facilitate detection by flow cytometry, and inserted into a 7-retroviral vector encoding for either CD28t or 4-1BBt signaling domains (FIG. 4) to generate the constructs [B7-H3-01]28t, [B7-H3-O1]BBt, [B7-H3-02]28t, and [B7-H3-02]BBt. Primary human T cells were isolated from healthy donors and were transduced to express the engineered CAR; no significant differences were observed in the ability of T cells to express any of the CARs and detected strong expression of each CAR (FIGS. 5 and 6). The cytolytic capacity of the differing B7-H3 targeting CAR T cells was evaluated through a 24-hour, luciferase-based CTL assay against B7-H3+U87 and U251 GBM cell lines, wherein tumor cells were cocultured with CAR T cells at decreasing effector to target ratios. To account for T cell alloreactivity, donor matched T cells were engineered to express the CD19 targeting CARs 1929t or 19BBt, and were used as negative controls. B7-H3 targeting CAR T cells derived from either [B7-H3-01] or [B7-H3-02] demonstrated robust cytolytic activity with minimal variation in ability to eliminate either cell line (FIGS. 7A-7C). Deeper analysis of the cytolysis data demonstrated that there were no observable differences when comparing [B7-H3-01]28t and [B7-H3-O1]BBt or [B7-H3-02]28t and [B7-H3-02]BBt CAR T cells, nor were there observables difference when comparing [B7-H3-01]28t and [B7-H3-02]28t or [B7-H3-O1]BBt and [B7-H3-02]BBt CAR T cells. The functional capabilities of the engineered B7-H3 targeting CAR T cells were further characterized by their ability to produce proinflammatory cytokines following coculture with B7-H3+ cells. B7-H3 or CD19 targeting CAR T cells were cocultured with U251, 3T3 cells engineered to express B7-H3, or 3T3 cells engineered to express CD19. Levels of effector cytokines IFNγ, TNFα, IL-2, and GM-CSF in supernatant were quantified after a 24-hour coculture, and fold change was determined relative to the level of the cytokines produced by CAR T cells alone. CAR T cells expressing scFv [1B7-H3-02] produced greater levels of IFNγ, TNFα, GM-CSF, and IL-2 in both the 28t and BBt as compared to the corresponding CAR T cells expressing scFv [1B7-H3-01](FIG. 19). [B7-H3-02]28t, outperformed [B7-H3-02]BBt by way of producing higher levels of proinflammatory cytokines. Accordingly, [B7-H3-02]28t was selected as the lead candidate for in vivo pharmacokinetic and pharmacodynamic evaluations, and designated as “GBM28t.”

To characterize the therapeutic capabilities of GBM28t we developed a disease-relevant orthotopic model wherein 1×10⁵ ffLuc tagged, U251 cells were implanted into the cerebellum of NSG mice, and after 14 days mice were treated with a single intravenous administration of 2.5×10⁶ GBM28t CAR T cells or 2.5×10⁶ 1928t CAR T cells as a control (FIG. 9A). Antitumor response was evaluated by quantifying longitudinal changes in photon flux from ffLuc BLI imaging; and GBM28t was observed to confer robust control of disease (FIG. 9C). Importantly, this decrease in tumor burden directly correlated with improved survival outcomes seen in the GBM28t treated cohort (FIG. 9D).

The presently disclosed subject matter also employed an in vivo stress-test wherein tumor bearing mice are treated with systematically decreasing numbers of CAR T cells to determine the minimum effective dose. Following xenograft mice were treated with either 2.5×10⁶, 1.0×10⁶, or 2.5×10⁵ GBM28ζ CAR T cells or 2.5×10⁶ 1928ζ CAR T cells as a control and responses were determined as before. GBM28ζ was observed to confer robust control of disease progression at the lowest dose, and complete responses at the higher dose levels (FIGS. 9H-9K). Strikingly, the cohort treated with 2.5×10⁵ GBM28ζ CAR T cells presented with a median survival of 72 days, nearly triple that of nontreated (27 days) and double that of 1928ζ (37 days) treated cohorts; while median survival was not reached in cohorts treated with the higher dose levels (FIG. 9L). Toxicity of GBM28ζ CAR T cells was also evaluated to interrogate for off-target effects through longitudinally observing animal weight. GBM28ζ CAR T cells did not demonstrate any toxicity following adoptive transfer as no cohorts apart from the nontreated and 1928ζ treated groups demonstrated meaningful weight loss, and this was attributed to disease progression (FIG. 9M).

CAR T cell efficacy was further quantified by evaluating the ability of CAR T cells to effectively home to disease sites. A bicistronic 7-retroviral construct was engineered to encode GBM28ζ CAR and exterior Gaussia luciferase (xGLuc) to longitudinally evaluate biodistribution and potential in vivo expansion of adoptively transferred CAR T cells (FIG. 10A). xGLuc produces a bioluminescent signal upon hydrolysis of its coelenterazine substrate, but most importantly is nonreactive towards luciferin. Using orthogonal substrates allows for imaging of both CAR T cells and ffLuc-tagged tumor cells within the same mouse. Moreover, given the rapid in vivo clearance of coelenterazine, images can be recorded as frequently as every 24 hours. 1×105 ffLuc tagged, U251 cells were orthotopically implanted into the cerebellum of NSG mice, and after 14 days mice were treated with a single intravenous administration of either 2.5×10⁶, 1.0×10⁶, or 2.5×10⁵ GBM28ζ-xGLuc CAR T cells or 2.5×10⁶ 1928ζ-xGLuc CAR T cells as a control. The presently disclosed subject matter observed rapid accumulation of GBM28ζ-xGLuc CAR T cells at disease sites within 24 hours of administration, and observed enrichment of the CAR T cells over the course of 7 days following administration (FIG. 10B) with maximum CAR T cell flux observed at 7 days after administration. Using a similar in vivo model, trafficking of CAR T cells to orthotopic sites was also evaluated by IHC. Mice were euthanized 21 days after adoptive transfer of CAR T cells, tumors were excised, fixed, embedded, sectioned and stained. Increased accumulation of human CD45+CD3+CD4+ and CD8+ populations in GBM28ζ treated cohorts was observed as compared to 1928ζ cohorts.

The presently disclosed subject matter demonstrated that targeting B7-H3 with CAR T cells is an effective strategy for therapy of glioblastoma. Employing a panel of scFvs derived from clinically validated antibodies generically fused to differential signaling domains, a CAR T cell vector was identified through multiple orthogonal, yet complimentary assays. CD28-based CARs demonstrated stronger in vitro efficacy profiles and one was selected as a lead candidate. The construct termed GBM28ζ demonstrated robust and reproducible expression in primary human T cells, as measured by flow cytometry. Moreover, GBM28ζ demonstrated robust activity against multiple GBM cell lines as measured by CTL assays and Luminex-based quantification of cytokine release. Together, these assays characterize each part of the CAR molecule, as antigen recognition by the scFv is required for receptor activation. Cytolytic activity is a readout of CD3ζ chain signaling, while TH1 cytokine production is representative of an active costimulatory domain. Utilizing an orthotopic xenograft model of glioblastoma, in vivo pharmacodynamic and pharmacokinetic profiles were characterized. An in vivo stress-test of GBM28ζ CAR T cells demonstrated robust activity in this model of GBM as measured by overall survival and reduction in tumor burden below the limit of detection at higher dose levels. Following systemic administration, GBM28ζ were observed to traffic to and enrich at orthotopic disease sites, suggesting that clinical translation of this moiety can be achieved without the need for stereotaxic surgery.

### Example 4

The presently disclosed subject matter first investigated if xenograft models of GBM recapitulate the preliminary observations from clinical literature. 1×10⁵ ffLuc tagged, U251 cells were orthotopically implanted into the cerebellum of NSG mice, and after 14 days mice were treated with a single intravenous administration of either 2.5×10⁶ GBM28ζ CAR T cells or 2.5×10⁶ 1928ζ CAR T cells as a control. Mice were euthanized 21 days after adoptive transfer of CAR T cells, tumors were excised, fixed, embedded, sectioned, and stained for various markers of T cell presence and activity. We observed increased staining of human PD-L1 in GBM28ζ treated cohorts as compared to 1928ζ treated, or nontreated cohorts (FIG. 12C). To expand upon this observation, an in vitro assay was developed to recapitulate the in vivo environment. U251, U87, 1108, and 0219 were used as representative samples from our panel of GBM cell lines, and these cells were exposed to differentially conditioned media, supplemented with recombinant IFNγ, TNFα, a combination of these cytokines, or activated T cell conditioned media (FIG. 11D). Following 72 hours of treatment PD-L1 expression was measured by flow cytometry, and induction of PD-L1 on each of the screened GBM cell lines was observed (FIG. 11E). While the level of induction was variable across the responding lines, ranging from 2-fold to 50-fold, each line responded similarly in respect to each condition (FIG. 11F). Data from the TCGA GBM dataset as compared to healthy brain tissue were also analyzed to investigate if PD-L1 was expressed at baseline by patients tumors and no increased PD-L1 expression was observed in GBM samples as compared to healthy brain tissue (data not shown).

Next, the expression of PD-1 on tumor infiltrating CAR T cells was interrogated. Specifically, if xenograft models of GBM recapitulated the preliminary observations from clinical literature that demonstrate upregulation of PD-1 on the surface of CAR T cells following administration. To avail recovery of tumor material and associated infiltrate, the presently disclosed xenograft system was modified to utilize a flank model in place of an orthotopic model. 1×10⁶ ffLuc tagged, U251 cells were subcutaneously implanted in the right flank of NSG mice, and after 14 days mice were treated with a single intravenous administration of 1.0×10⁶ GBM28ζ CAR T cells. Mice were euthanized 21 days after adoptive transfer of CAR T cells, tumors were excised, dissociated, stained, and analyzed by flow cytometry. For comparison, spleens from these mice were also recovered and dissociated to isolate peripheral, non-tumor infiltrating CAR T cells. Increased PD-1 staining was observed on tumor infiltrating CAR T cells as compared to peripheral CAR T cells (FIGS. 12B, 12C, 12G, and 12H).

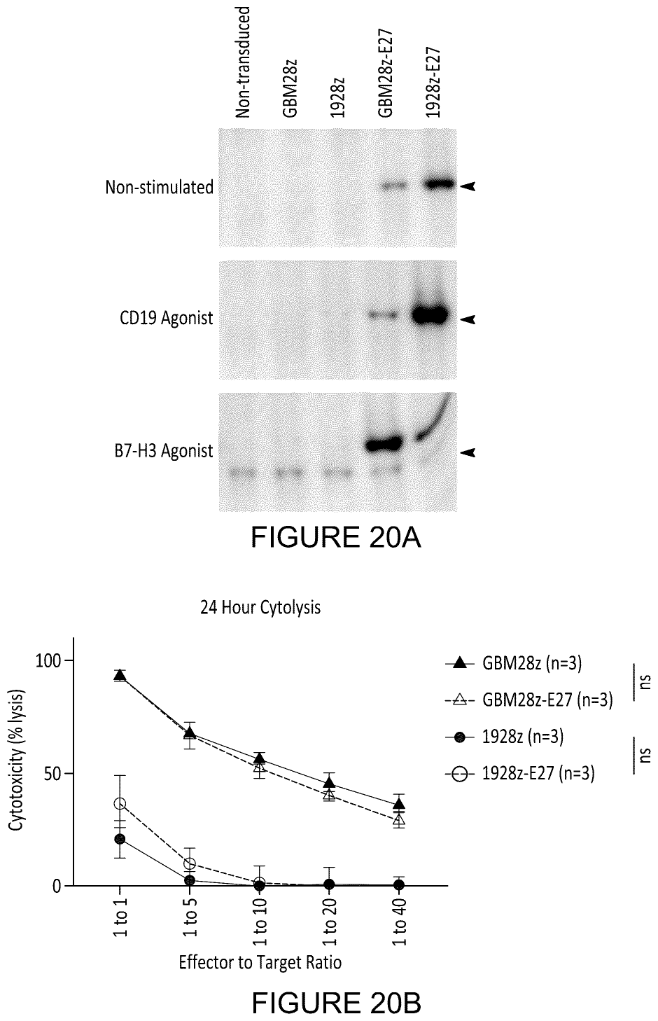

Having demonstrated that GBM tumor cells increase surface expression of PD-L1 in response to T cell activity, and that GBM28ζ demonstrates increased surface expression of PD-1 following activation through the CAR, it was hypothesized that combinatorial PD-1 blockade could augment in vivo antitumor efficacy of GBM28ζ CAR T cells. GBM28ζ vectors were modified to carry the PD-1 blocking scFv referred to as “E27” (FIG. 13A). It was demonstrated robust and reproducible expression of the E27 armored construct in primary human T cells (FIG. 13B). By taking advantage of the orthogonal epitope tags in the E27 scFv it was possible to discern levels of E27 in cell culture supernatant at basal and under stimulated conditions. As measured by HA staining, we observed stable production of E27 under resting conditions and increased levels following stimulation through the CAR (FIG. 20A). Next, it was interrogated the ability for E27 to augment CAR T cell cytolytic capacity; and through an in vitro luciferase-based CTL assay demonstrated concomitant PD-1 blockade to not alter in vitro cytolytic ability of CAR T cells (FIG. 20B).

To evaluate the therapeutic capabilities of GBM28ζ-E27 as compared to GBM28ζ, an in vivo stress test was employed wherein tumor bearing mice were treated with systematically decreasing numbers of CAR T cells to determine the minimum effective dose. 1×10⁵ ffLuc tagged, U251 cells were orthotopically implanted into the cerebellum of NSG mice, and after 21 days mice were treated with a single intravenous administration of either 1.0×10⁶, or 2.5×10⁵ GBM28ζ-E27 CAR T cells or a matched dose of GBM28ζ CAR T cells as a control. Antitumor response was evaluated by quantifying longitudinal changes in photon flux from ffLuc BLI imaging. GBM28ζ-E27 was observed to confer improved survival at both dose levels, while the lower dose level demonstrated a statistically significant survival advantage (FIGS. 16-16C). The cohort treated with 2.5×10⁵ GBM28ζ-E27 CAR T cells presented with a median survival of 62 days, a 33 percent increase as compared to the 46 day median survival of GMB28z treated or nontreated cohorts; similar to earlier experiments, median survival was not reached cohorts treated with the higher dose levels.

It was then sought to characterize the mechanism by which concomitant PD-1 blockade augments CAR T cell function. Like earlier experiments, xenograft systems were modified to facilitate recovery of tumor material by utilizing a subcutaneous flank model in place of an orthotopic model. 1×10⁶ ffLuc tagged, U251 cells were implanted subcutaneously in the right flank of NSG mice, and after 21 days mice were treated with a single intravenous administration of either 1.0×10⁶ GBM28ζ-E27 CAR T cells or a matched dose of GBM28ζ CAR T cells as a control. Mice were euthanized 14 days after adoptive transfer of CAR T cells, tumors were excised, dissociated, stained, and analyzed by flow cytometry. At this timepoint, no differences were observed in tumor size (mass) between the differentially treated groups, however, we observed increased levels of intratumoral CAR T cells in mice which received GBM28ζ-E27 as compared to those mice which received GBM28ζ. Further profiling of tumor infiltrating CAR T cells demonstrated differences in memory population, but not in expression of exhaustion markers. Intratumoral CAR T cells recovered from the GBM28ζ-E27 treated cohort demonstrated a greater fraction of effector memory cells (CD45RA−, CD62L−) as compared to the GBM28ζ treated cohort. Furthermore, quantification of surface PD-1 levels on intratumoral CAR T cells did not reveal a difference between the differentially treated cohorts, and the same was observed on peripheral CAR T cells isolated from the spleen of the differentially treated cohorts. See FIGS. 17A-17E. Together these data indicate that concomitant PD-1 blockade delivered through a secretable scFv does not augment efficacy through simply driving the cells to express lower levels of PD-1.

The presently disclosed subject matter demonstrated that blockade of the PD-1/PD-L1 checkpoint pathway is an effective strategy to augment the efficacy of B7-H3 targeting CAR T cell therapy of GBM. Early results from clinical investigations employing CAR T cells to target glioblastoma hinted that a major mechanism of resistance employed by tumor cells was upregulation of PD-L1 with concomitant upregulation of the cognate receptor PD-1 on the adoptively transferred product. Analysis of tumor RNAseq data did not demonstrate baseline enrichment of PD-L1 expression by patient GBM tissue as compared to healthy brain tissue. Employing a murine xenograft model which faithfully recapitulates CAR T cell and GBM tumor interactions demonstrated that this pathway was indeed a major mechanism of resistance to our B7-H3 targeted CAR T cell platform. Moreover, PD-L1 was identified to be upregulated by independently derived primary GBM cell lines following exposure to proinflammatory milieu derived from activated CAR T cells. Investigating the therapeutic potential of combinatorial B7-H3 targeting CAR T cells with PD-1 blockade demonstrated increased survival benefits within orthotopic xenograft models of GBM. It was demonstrated that this effect was conferred through augmented infiltration of GBM tumors by CAR T cells, and that these cells were less terminally differentiated than monotherapy cohorts. By taking advantage of the CAR T cell to act as a micropharmacy, it was demonstrated that PD-1 blockade by way of a CAR T cell engineered to secrete a PD-1 blocking scFv to be advantageous.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising an antigen-recognizing receptor comprising an extracellular antigen-binding domain, a transmembrane domain, and an intracellular signaling domain, wherein the extracellular antigen-binding domain specifically binds to B7-H3 and comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

2. The immunoresponsive cell of claim 1, wherein the extracellular antigen-binding domain comprises a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

3. The immunoresponsive cell of claim 1, wherein the extracellular antigen-binding domain is a single-chain variable fragment (scFv), a Fab, or a F(ab)₂.

4. The immunoresponsive cell of claim 1, wherein the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, ACTIVE 508624478.1 1 about 96%, about 97%, about 98% or about 99% homologous or identical to the amino acid sequence selected set forth in SEQ ID NO: 7 or SEQ ID NO: 17; and/or (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or SEQ ID NO: 18.

5. The immunoresponsive cell of claim 4, wherein the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8; or
(b) a heavy chain variable region comprising the amino acid sequence set forth in SEQ ID NO: 17, and a light chain variable region comprising the amino acid sequence set forth in SEQ ID NO: 18.

6. The immunoresponsive cell of claim 1, wherein the extracellular antigen-binding domain comprises a linker between a heavy chain variable region and a light chain variable region of the extracellular antigen-binding domain.

7. The immunoresponsive cell of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

8. The immunoresponsive cell of claim 1, wherein the intracellular signaling domain comprises a CD3ζ polypeptide.

9. The immunoresponsive cell of claim 8, wherein the CD3ζ polypeptide comprises or consists of the amino acid sequence set forth in SEQ ID NO: 43.

10. The immunoresponsive cell of claim 8, wherein the intracellular signaling domain further comprises at least one co-stimulatory signaling region.

11. The immunoresponsive cell of claim 10, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

12. The immunoresponsive cell of claim 11, wherein the at least one co-stimulatory signaling region comprises a CD28 polypeptide or a 4-1BB polypeptide.

13. The immunoresponsive cell of claim 12, wherein the CD28 polypeptide comprises a mutated YMNM motif.

14. The immunoresponsive cell of claim 13, wherein the CD28 polypeptide comprises or consists of the amino acid sequence set forth in SEQ ID NO: 96, SEQ ID NO: 97, SEQ ID NO: 98, SEQ ID NO: 99, or SEQ ID NO: 100.

15. The immunoresponsive cell of claim 1, wherein the antigen-recognizing receptor is a chimeric antigen receptor (CAR), or a T-cell like fusion protein.

16. The immunoresponsive cell of 15, wherein the CAR comprises the amino acid sequence set forth in SEQ ID NO: 102, SEQ ID NO: 104, or SEQ ID NO: 106.

17. The immunoresponsive cell of 16, wherein the CAR comprises the amino acid sequence set forth in SEQ ID NO: 102.

18. The immunoresponsive cell of claim 1, further comprising a soluble scFv.

19. The immunoresponsive cell of claim 18, wherein the soluble scFv comprises a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

20. The immunoresponsive cell of claim 19, wherein the soluble scFv comprises (a) a heavy chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 27; and/or (b) a light chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 28.

21. The immunoresponsive cell of claim 20, wherein the cell comprises a polypeptide comprising an amino acid sequence that is at least about 85%, about 90%, about 95%, about 96%, about 97%, about 98%, or about 99% to the amino acid sequence set forth in set forth in SEQ ID NO: 112.

22. The immunoresponsive cell of claim 21, wherein the cell comprises a polypeptide comprising the amino acid sequence set forth in set forth in SEQ ID NO: 112

23. The immunoresponsive cell of claim 1, wherein the cell is (a) a cell of the lymphoid lineage or a cell of the myeloid lineage, (b) selected from the group consisting of a T cell, a Natural Killer (NK) cell, and a stem cell from which a lymphoid cell may be differentiated, or (c) a stem cell or a pluripotent stem cell.

24. The immunoresponsive cell of claim 23, wherein the T cell is a cytotoxic T lymphocyte (CTL) or a regulatory T cell.

25. A composition comprising the cell of claim 1.

26. The composition of claim 25, which is a pharmaceutical composition further comprising a pharmaceutically acceptable carrier.

27. A nucleic acid encoding:
(a) an antigen-recognizing receptor comprising an extracellular antigen-binding domain, a transmembrane domain, and an intracellular signaling domain, wherein the extracellular antigen-binding domain specifically binds to B7-H3 and comprises:
(i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
(ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; and
(b) a soluble scFv comprising a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

28. The nucleic acid of claim 27, wherein the nucleic acid encodes the amino acid sequence set forth in SEQ ID NO: 112.

29. A vector comprising the nucleic acid of claim 27.

30. A lipid nanoparticle comprising the nucleic acid of claim 27.

31. A method of producing an immunoresponsive cell targeting B7-H3, the method comprising introducing into the cell the nucleic acid of claim 27.

32. A method of treating or ameliorating a tumor or cancer in a subject, comprising administering to the subject an effective amount of the presently disclosed cell of claim 1.

33. The method of claim 32, wherein the tumor or cancer is selected from the group consisting of osteosarcoma, rhabdomyosarcoma, Ewing's sarcoma, neuroblastoma, desmoplastic small round cell tumor, synovial sarcoma, undifferentiated sarcoma, adrenocortical carcinoma, hepatoblastoma, Wilms tumor, rhabdoid tumor, high-grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma, head and neck cancers, leiomyosarcoma, and melanoma.

34. The method of claim 32, wherein the subject is a human.

35. A kit for treating or ameliorating a disease or disorder in a subject, comprising the cell of claim 1.
